(19) United States
(12) Patent Application Publication
(10) Pub. No.: US 2025/0195409 A1
(43) Pub. Date: Jun. 19, 2025

(54) METHODS FOR IMPROVING CUTANEOUS PERMEATION OF CANNABINOIDS AND FATTY ACID AMIDES

(71) Applicant: ANANDA SCIENTIFIC, INC., Greenwood Village, CO (US)

(72) Inventor: Mark J. Rosenfeld, Draper, UT (US)

(73) Assignee: Ananda Scientific, Inc., Greenwood Village, CO (US)

(21) Appl. No.: 18/554,605

(22) PCT Filed: Apr. 11, 2022
(86) PCT No.: PCT/US22/24276

§ 371 Date: Oct. 9, 2023

Related U.S. Application Data
(60) Provisional application No. 63/173,204, filed on Apr. 9, 2021.

(51) Int. Cl. A61K 9/00; A61K 9/06; A61K 45/06; A61K 47/22; A61P 29/00

(52) U.S. Cl. CPC A61K 9/0014; A61K 9/06; A61K 45/06; A61K 47/22; A61P 29/00

(57) ABSTRACT

The present disclosure is drawn to compositions and methods for enhancing transdermal administration. In one embodiment, a topical composition can comprise a therapeutically effective amount of at least one cannabinoid receptor modulator. In another aspect, the topical composition can comprise an amount of a combination of a first and second penetration enhancer at synergistic ratios that facilitate a greater combined therapeutic effect than provided by a sum of individual therapeutic effects of the individual components of the combination. In another aspect, the topical composition can comprise a pharmaceutically acceptable carrier suitable for topical administration.

### PRIORITY DATA

This application claims the benefit of U.S. Provisional Application Ser. No. 63/173,204, filed on Apr. 9, 2021, which is incorporated herein by reference.

### FIELD OF THE DISCLOSURE

The present disclosure relates to therapeutic compositions containing cannabinoids and fatty acid amides. Exemplary compositions can be formulated for topical administration and can be indicated for treatment of pain and inflammation among other things. Accordingly, this disclosure involves the fields of chemistry, pharmaceutical sciences, medicine, and other health sciences.

### BACKGROUND

Cannabinoids are naturally found in several plants within the Cannabis genus, and in particular in the species Cannabis sativa, Cannabis indicia, and Cannabis ruderalis. Two cannabinoid compounds, namely, tetrahydrocannabinol (THC) and cannabidiol (CBD) have been especially studied from these plants. However, other cannabinoids, specifically, cannabigerol and cannabichromene along with cannabinol, a metabolite derived from tetrahydrocannabinol, have more recently received scientific and medical attention.

Cannabinoids have been used for many years in a primarily unregulated manner both for therapeutic and recreational purposes. While cannabinoids might be potentially therapeutic for a number of ailments and conditions, one barrier to therapeutic implementation is the difficulty in administering cannabinoids in an efficient and effective manner through certain delivery routes. Of course, one convenient and user-friendly form of administration for certain indications such as muscular or joint pain and peripheral neuropathy as well as anti-inflammation is the topical route of administration. Unfortunately, many cannabinoids, such as cannabidiol, have chemical properties, such as size, hydrophobicity, etc. which makes administration of therapeutically effective amounts without the assistance of a penetration enhancer very challenging. In view of the potential benefits of efficient and effective topical cannabinoid therapy, compositions and formulations which achieve improved cannabinoid skin penetration and delivery continue to be sought.

### SUMMARY

The present disclosure is drawn to compositions and methods for enhancing transdermal administration of cannabinoids and similar agents that action on cannabinoid receptors to provide therapeutic benefits. In one embodiment, a topical composition can comprise a therapeutically effective amount of at least one cannabinoid receptor modulator. In another aspect, the topical composition can comprise an amount of a combination of penetration enhancers (e.g. a first and second penetration enhancer) at synergistic ratios or amounts that facilitate a greater combined therapeutic effect than provided by a sum of individual therapeutic effects of the individual components of the combination. In another aspect, the topical composition can comprise a pharmaceutically acceptable carrier suitable for topical administration.

In one aspect, the cannabinoid receptor modulator can comprise a cannabinoid, a fatty acid amide, certain terpenes, a class of plant-derived products consisting of compounds with the formula (C₅H₈)ₙ, or a combination thereof. In another aspect, the fatty acid amide can comprise one or more of an N-acylethanolamine, a fatty acid primary amide (FAPA), or a combination thereof.

In one aspect, the first penetration enhancer, the second penetration enhancer, or both can be amphiphilic. In another aspect, the first penetration enhancer, the second penetration enhancer, or both can comprise at least one terpene. In another aspect, at least one terpene can be a cannabis extract, or the same from another plant. In another aspect, the first penetration enhancer, the second penetration enhancer, or both can comprise one or more of a hexadecenoic acid, a salt of hexadecenoic acid, an ester of hexadecenoic acid, or a combination thereof. In another aspect, the topical composition can comprise a secondary active agent.

In another embodiment, the topical composition can be formulated as one of: a solution, a suspension, an emulsion, a gel, a hydrogel, a thermo-responsive gel, a cream, a lotion, an ointment, a liniment, a paste, a mousse, an aerosol, a spray, a wax, a balm, a suppository, an adhesive, erodible matrix, a liquid reservoir, a patch (including an adhesive matrix patch), or a combination thereof.

In yet another embodiment, a method of treating a condition in a subject that is responsive to treatment with a cannabinoid receptor modulator can comprise administering a therapeutically effective amount of a topical composition as recited herein to the subject.

In another embodiment, a method of enhancing transdermal penetration of a cannabinoid receptor modulator can comprise: combining an amount of the cannabinoid receptor modulator with a combination of first and second penetration enhancers at synergistic amounts or ratios that achieve a greater combined therapeutic effect than provided by a sum of individual therapeutic effects of the individual components of the combination. In one aspect, the combination can facilitate an extended therapeutic effect.

### DETAILED DESCRIPTION

Before disclosure embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting.

Furthermore, the described features, structures, or characteristics can be combined in any suitable manner in one or more embodiments. In the following description, numerous specific details are provided, such as examples of compositions, dosage forms, treatments, etc., to provide a thorough understanding of various disclosure embodiments. One skilled in the relevant art will recognize, however, that such detailed embodiments do not limit the overall disclosure concepts articulated herein, but are merely representative thereof.

### Definitions

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As used herein, the terms “treat,” “treatment,” or “treating” and the like refers to administration of a therapeutic agent to a subject who is either asymptomatic or symptomatic. In other words, “treat,” “treatment,” or “treating” can refer to the act of reducing or eliminating a condition (i.e., symptoms manifested), or it can refer to prophylactic treatment (i.e., administering to a subject not manifesting symptoms in order to prevent their occurrence). Such prophylactic treatment can also be referred to as prevention of the condition, preventative action, preventative measures, and the like.

As used herein, the terms “therapeutic agent,” “active agent,” and the like can be used interchangeably and refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation in this written description is intended to include the active agent per se, as well as provide express support for pharmaceutically acceptable salts, esters or compounds significantly related thereto, including without limitation, prodrugs, active metabolites, isomers, and the like. Moreover, when a specific form of a compound such as a salt, ester, prodrug, metabolite, isomer, etc., is recited in this written description, it is to be understood that such recitation also provides express support for the active agent per se (e.g. free base) or other well-known forms of the active agent.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format for administration to a subject. For example, an “oral dosage form” can be suitable for administration to a subject's mouth. A “topical dosage form” can be suitable for administration to a subject's skin by rubbing, etc.

As used herein, “pharmaceutically acceptable carrier” or “carrier” are used interchangeably and refer to a pharmaceutical acceptable agent that can be capable of fully or partially dissolving or solubilizing an active agent (e.g., cannabidiol) in the pharmaceutical composition. Further, in some aspects, the carrier can be an agent that can be varied for the alteration of release rate and/or extent of the active agent from the composition and/or the dosage form. Further, the carrier may be polymeric, such as an adhesive, or non-polymeric, and is generally admixed with other components of the composition (e.g., drug, binders, fillers, penetration enhancers, anti-irritants, emollients, lubricants, etc.) to comprise the formulation. The term “admixed” means that the drug and/or other ingredients can be dissolved, dispersed, or suspended in the carrier. In some cases, the drug may be uniformly admixed in the carrier.

As used herein, “cannabinoid” or “cannabinoids” refers to one or more compounds found in a Cannabis plant, such as the species of Cannabis sativa, Cannabis indicia, and Cannabis ruderali, including hemp plants, such as industrial hemp. Notable cannabinoids include tetrahydrocannabinol (THC) (Delta9-THC or Delta8-THC), and cannabidiol (CBD). Other cannabinoids typically obtained from Cannabis plants include cannabidgerol, cannabichromene and cannabinol, a degradation product of tetrahydrocannabinol. Cannabidiol has the general structure:

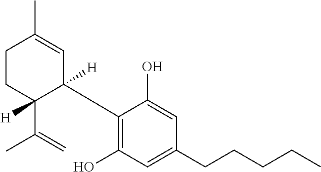 [SMILES: C=C(C)[C@@H]1CCC(C)=C[C@H]1c1c(O)cc(CCCCC)cc1O]

and is known under the TUPAC name 2-[(1R,6R)-3-methyl-6-prop-1-en-2-ylcyclohex-2-en-1-yl]-5-pentylbenzene-1,3-diol. It should be understood that recitation of the term “cannabinoid” in this written description provides express support for each and every pharmacologically active cannabinoid species, including CBD, THC, and/or other cannabinoids.

As used herein, the term “fatty acid” refers to unionized carboxylic acids with a long aliphatic tail (chain), either saturated or unsaturated, conjugated or non-conjugated.

As used herein, the term “fatty acid amide” refers to an amide formed from a fatty acid and an amine. In one example, a “fatty acid amide” can comprise the structure: RC(O)N(H)CH₂CH₂OH. In another example, a “fatty acid amide” can comprise the structure: RC(O)NH₂.

As used herein, the term “soluble” is a measure or characteristic of a substance or agent with regards to its ability to dissolve in a given solvent. The solubility of CBD in a particular component of the composition refers to the amount of the CBD dissolved to form a visibly clear solution at a specified temperature such as about 25° C. or about 37° C.

As used herein, the term “lipophilic,” refers to compounds that are not freely soluble in water; and the term “lipophilic surfactant” refers to surfactants that have HLB values of about 10 or less. Conversely, the term “hydrophilic” refers to compounds that are soluble in water; and the term “hydrophilic surfactant” refers to surfactants that have HLB values of more than about 10.

As used herein, a “semi-liquid” corresponds to a partially solubilized active agent and a “liquid” corresponds to a fully solubilized active agent.

As used herein, “solid” and “semi-solid” refers to the physical state of a composition that supports its own weight at standard temperature and pressure and has adequate viscosity or structure to not freely flow. Semi-solid materials may conform to the shape of a container under applied pressure.

As used herein, a “subject” refers to a mammal that may benefit from the administration of a drug composition or method disclosed herein. Examples of subjects include humans, and may also include other animals such as horses, pigs, cattle, dogs, cats, rabbits, and aquatic mammals. In one specific aspect, a subject is a human. In another aspect, the subject is a female. In another aspect, the subject is male.

As used herein, “in need of treatment” refers to a subject that has a disease or is suspected of having the disease according to various diagnostic criteria typically used in practice, or desires treatment or is indicated for treatment. Thus, “in need of treatment” can include the operation of identifying a subject in need of treatment.

As used herein, “identifying a subject in need of treatment” can include the operation of obtaining a biological sample from the subject and determining the level of one or more biomarkers as described herein, assessing a biological sample obtained from said subject, performing an imaging analysis on the subject, assessing one or more clinical characteristics of said subject (e.g., assessing symptoms or overt symptoms), or a combination thereof.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, inflammation, diabetes, hypertension, high cholesterol, and the like.

As use herein with respect to physiologic levels of a given substance, the term “baseline” refers to a level or concentration of the substance in a subject prior to administration of an active agent. For example, the baseline level of CBD in a subject would be the subject's CBD serum level prior (e.g., just prior) to the commencement of CBD administration or therapy.

As used herein, the terms “release” and “release rate” are used interchangeably to refer to the discharge or liberation of a substance, including without limitation a drug, from the dosage form into a surrounding environment such as an aqueous medium either in vitro or in vivo.

In some aspects of the present disclosure, the release of the drug may be controlled release. As used herein, the term “controlled release” represents the release of the drug from the dosage form according to a profile that differs from an unrestricted or uncontrolled release profile, such as a predetermined profile. In some aspects, the controlled release selected can be, intermediate, delayed, extended, sustained, or pulsatile. In another aspect, combinations of the aforementioned release profiles may be used in order to achieve specific delivery results, such as an immediate release followed by a delayed and/or a sustained release of the active agent.

As used herein, a “dosing regimen” or “regimen” such as an “initial dosing regimen” or “starting dose” or a “maintenance dosing regimen” refers to how, when, how much, and for how long a dose of the compositions of the present disclosure can be administered to a subject. For example, an initial or starting dose regimen for a subject may provide for a total daily dose of from about 5 mg/kg of body weight to about 50 mg/kg of body weight.

As used herein, “daily dose” refers to the amount of active agent (e.g., cannabidiol) administered to a subject over a 24-hour period of time. The daily dose can be administered as one or more administrations during the 24-hour period. In one embodiment, the daily dose provides for two administrations or more in a 24-hour period.

As used herein, “synergistic” means more than the additive effect of the individual components of a composition or formulation against a mechanism of action (e.g. penetration of a drug through the skin). For example if F1 produces response X, F2 produces response Y, then the combination of F1+F2>X+Y. In some situations F2 produces no response and the value for Y is equal to zero.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or by other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine.

As used herein “single unit” when used to describe dosing of a subject refers to the dosage form being a single dosage form, e.g. a single tablet, capsule, pump or squirt of gel or solution, etc. In contrast, “multiple unit” when used to describe dosing of a subject refers to the dosage including two or more dosage forms, e.g. 2 capsules, 3 tablets, 2-4 pumps or squirts, one patch, etc. It is noteworthy that multiple unit dosage forms generally will be the same type of dosage forms (i.e., lotion or creme) but are not required to be the same dosage form type.

As used herein, a “treatment situs” refers to a location on or within a subject where treatment is desired. For example, when treating a burn or a lesion, the treatment situs can be the area of the burn or lesion. When treating a respiratory condition affecting the lungs, the treatment situs can be the lungs. Further, as used herein, an “application situs” refers to a location on or in a subject where treatment is administered. For example, the application situs for an oral dosage formulation may be the subject's mouth. Further, the application situs for an infusion dosage formulation may be an area where the infusion equipment enters the subject's circulatory system. Yet further, the application situs for a topical dosage formulation may be the area of skin or mucosa to which the topical dosage formulation is applied. In some embodiments, the application situs may be substantially the same as the treatment situs (e.g., the composition or formulation is administered directly to the treatment site). In other embodiments, the application situs may be different from (e.g., distal from) the treatment situs. In such cases despite the fact that administration may be distal from the treatment situs, the composition or formulation still exerts a therapeutic effect at the treatment situs.

As used herein, “topical composition” or “topical administration” and the like refer to a composition suitable for administration directly to a skin or mucosa surface and from which an effective amount of a drug is released. In some embodiments, topical compositions can provide a local or localized therapeutic effect (e.g., at or near an application situs). For example, a topical composition when applied to a wound, a lesion, a burn, a canker sore, etc. (e.g., a treatment situs), may primarily exert a therapeutic effect at or around the application situs, but not substantially beyond it. In other embodiments, a topical composition can provide a regional effect. For example, a topical composition administered to a skin surface on a region of the body, such as a finger, arm, ankle, joint, etc. can exert a therapeutic effect within the region, but not substantially beyond. For example, a topical composition administered to the region of an ankle can have a therapeutic effect in and around the ankle, by for example, reducing edema, joint inflammation, pain, etc. In other embodiments, topical compositions can provide a systemic effect. In some aspects, a topical composition can provide the therapeutic effect though a mechanism of action where the drug or active agent itself arrives at the treatment situs. In other aspects, the topical composition can provide the therapeutic effect through an intermediate mechanism of action, such as biochemical cascade event, such as an enzymatic cascade or other signaling (e.g., cellular signaling, or inter/intra cellular signaling) event which ultimately exerts the desired therapeutic effect at the treatment situs. In some examples, such intermediate mechanism can allow treatment of a treatment situs that is distal from an application situs. In yet other examples, when treatment of a distal treatment situs occurs, the active agent may travel through dermal and other tissues from the application situs to the treatment situs and exert a direct effect.

As used herein, “transdermal” refers to the route of administration of a therapeutic agent through a skin surface when administered to the skin surface. Transdermal refers to non-invasively delivering over a period of time after application a substance or substances into the skin layers and underlying tissues and, sometimes from there, into the systemic circulation. When transdermally administered, the drug or active agent migrates from the application situs to a treatment situs and exerts a therapeutic effect. Transdermal compositions and dosage forms can include structures and/or devices which assist in holding the composition on a skin surface, such as, for example, backing films, adhesives, reservoirs, etc. Furthermore, transdermal compositions can include agents which aid or otherwise facilitate movement of the active agent from an application situs to a treatment situs (e.g., through the skin and into the subject's circulatory system) such as penetration or permeation enhancers. Such penetration or permeation enhancers can also be used with topical formulations in some embodiments.

Examples of transdermal formulations include, but are not limited to, ointments, creams, gels, transdermal patches, sprays, lotions, mousses, aerosols, nasal sprays, buccal and sublingual tablets and tapes or adhesives, vaginal rings, and pastes. The term “transdermal administration” thus refers to the transdermal application of a formulation (in some examples a topical formulation) or composition. Transdermal administration can be accomplished by applying, pasting, rolling, attaching, pouring, pressing, rubbing, etc., of a transdermal preparation or formulation onto a skin or mucosal surface of a subject.

The terms “transdermal delivery system,” “transdermal patches” or simply “patches” refer to a matrix or liquid reservoir type of transdermal delivery device which is used to transdermally deliver defined doses of a substance, over a specific application period.

By the term “matrix”, “matrix system”, or “matrix patch” is meant a composition comprising an effective amount of a drug dissolved or dispersed in a polymeric phase, often a pressure sensitive adhesive, which may also contain other ingredients, such as a penetration enhancers, skin irritation reducing agents, excipients, plasticizers, emollients, and the like. This definition is meant to include embodiments wherein such polymeric phase is laminated to a pressure sensitive adhesive or used within an overlay adhesive.

The general structure of a matrix-type patch can include a drug-impermeable occlusive backing laminated to the distal side of a solid or semisolid matrix layer comprised of a homogeneous blend of the drug, a polymeric pressure sensitive adhesive carrier, and optionally one or more skin penetration enhancers, and a temporary peelable release liner adhered to the proximal side of the matrix layer. In use, the release liner is removed prior to application of the patch to the skin.

Additionally, the general structure of a liquid reservoir system (LRS) type patch can comprise a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. The drug and any penetration enhancers can be contained in the fluid in desired amounts. For application, a peelable release liner is removed and the patch is attached to the skin surface

As used herein, “enhancement,” “penetration enhancement,” or “permeation enhancement,” refer to an increase in the permeability of the skin to a drug, so as to increase the rate at which the drug permeates through the skin. Thus, “permeation enhancer,” “penetration enhancer,” or simply “enhancer” refers to an agent, or mixture of agents that achieves such permeation enhancement.

The term “skin” or “skin surface” includes not only the outer skin of a subject comprising one or more epidermal layers, but also mucosal surfaces such as the mucosa of the respiratory (including nasal and pulmonary), oral (mouth and buccal), vaginal, and rectal cavities. Hence, the term “transdermal” may encompass “transmucosal” as well.

As used herein, “co-administering” a first therapeutic agent with a second therapeutic agent can include concomitant administration within a suitable time window. In one example, the suitable time window can be less than one or more of: 1 hour, 45 minutes, 30 minutes, 15 minutes, 5 minutes, 2 minutes, 1 minute, or combinations thereof. Concomitant administration can be from the same composition or from different compositions.

In this disclosure, “comprises,” “comprising,” “containing” and “having” and the like can have the meaning ascribed to them in U.S. Patent law and can mean “includes,” “including.” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment.” or “in one aspect,” herein do not necessarily all refer to the same embodiment or aspect.

As used herein, comparative terms such as “increased,” “decreased,” “better,” “worse,” “higher” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, a composition that “increases” CBD serum levels provides a CBD level in a subject that is elevated as compared to a serum level at a previous point in time, such as a baseline level (e.g., prior to treatment), or as compared to an earlier treatment with a different (e.g., lower dose).

As used herein, the term “substantially;” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defcro equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Units of measure of amounts or concentrations can be expressed herein by any suitable and recognized quantitation or output, such as milligrams (mg), milliliters (ml), etc. Individual ingredients or agents can also be expressed in relation to other ingredients or agents, or combinations of ingredients or agents, such as a composition or formulation. For example, the concentration or amount of an ingredient or agent can be articulated in terms of its percentage by weight (e.g. weight percent, or percent weight, wt %) of the composition or formulation. Unless the context dictates otherwise, when using terms such as wt % in this disclosure, the amount of the identified ingredient or agent will be its percentage by weight of the composition or formulation, or other sub-formulation or mixture combination identified. Additionally, numerical values expressed in terms of ratios will be in terms of weight percentage (wt %) ratios unless otherwise state expressly or by context.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### DESCRIPTION

Reference will now be made in detail to embodiments of the disclosure. While the disclosure will be described in conjunction with the embodiments, it will be understood that it is not intended to limit the disclosure to those embodiments. To the contrary, it is intended to cover alternatives, variants, modifications, and equivalents as may be included within the spirit and scope of the disclosure as defined by the appended claims.

An initial overview of technology embodiments is provided below, and then specific technology embodiments are described in further detail later. This initial summary is intended to aid readers in understanding the technology more quickly but is not intended to identify key features or essential features of the technology nor is it intended to limit the scope of the claimed subject matter.

In living cells, a receptor refers to an organized assemblage of protein molecules on the outer cell surface or inside the cell to which a particular substance such as a hormone, a foreign substance, a medicament or an antigen will bind, thus instigating a modification in the activity of that particular cell. Substances that bind to the receptor and turn on a process are termed “direct agonists”. Indirect agonists may bind to a particular receptor and cause the manufacture of a particular molecule. In turn, that molecule may bind to another receptor and consequently turn on a process. “Indirect agonists” can achieve their effect not only by causing the synthesis of a compound that in turn causes processes in another receptor, but also through other mechanisms that can influence receptor function like transporter blockade, the induction of transmitter release, and inhibition of transmitter breakdown. Whether direct or indirect, modulation of receptors can be induced to create desired physiological effects.

A particular cannabinoid, one of a group of closely related chemical moieties that come from plants in the genus Cannabis can be a direct or indirect agonist. In vertebrates, endocannabinoid receptors are of two sorts: one receptor makes the endocannabinoid, anandamide, and the other receptor makes the endocannabinoid, 2-arachidonoylglycerol. The cannabinoid, delta-9-tetrahydrocannabinol, is a direct agonist of the receptor that makes anandamide. Delta-9-tetrahydrocannabinol can attach to the receptor and cause anandamide synthesis.

In contrast, the cannabinoid, cannabidiol, does not bind to either of the endocannabinoid receptors; therefore, cannabidiol does not cause anandamide synthesis but instead inactivates the enzyme that breaks down anandamide to increase anandamide accumulation. Thus, cannabidiol is an indirect agonist of the endocannabinoid receptor responsible for anandamide. There are non-cannabinoids that can impact the endocannabinoid receptors. Fatty acid amides, amides formed from a fatty acid and an amine, like palmitoylethanolamide can function as indirect agonists of the endocannabinoid system.

Cannabinoids are virtually insoluble in water but can be dissolved in fat and alcohol. The efficient topical or transdermal delivery of cannabinoids can be hindered because the lipophilic properties of cannabinoids can restrict permeation when cannabinoids are placed directly onto the skin. Therefore, enhancing skin permeation can be useful with topical or transdermal administration of cannabinoids and fatty acid amides.

Palmitoylethanolamide is an exogenous fatty acid amide that can play a role in intracellular signaling. Palmitoylethanolamide is highly lipophilic and thus fails to to achieve efficient skin permeation. Palmitoylethanolamide can function in connection with cannabinoids by modulating endocannabinoid signaling via activation, although indirectly instead of directly, of the same cannabinoid receptors. Palmitoylethanolamide biological activity resembles that of anandamide. Palmitoylethanolamide also works through the peroxisome proliferator-activated receptor alpha, a nuclear receptor protein, as well as via an affinity to cannabinoid-like G-coupled receptors GPR55 and GPR119, through which it manifests varied biological effects, some related to chronic inflammation and pain.

Therefore, enhancing skin permeation can be useful with topical or transdermal administration of cannabinoids and fatty acid amides.

Increasing the bioavailability of active agents can enhance the efficacy of dermal or topical formulations. Penetration or permeation enhancers, also called sorption promoters or accelerants are used to do this. Penetration enhancers are chemically-related substances that can reversibly compromise the stratum comeum, or outermost epidermal layer, of the skin and accordingly facilitate the entry of poorly permeating substances (transdermal permeation), or amplify movement into the skin along hair follicles, sweat glands and sebaceous glands (transappendaged permeation). While skin penetration can be increased, the extent to which each active agent can or will permeate can be variable. Thus, penetration enhancers have limits in moving active agents across the stratum corneum and deeper into the skin.

In particular, isosorbide dimethyl ether (dimethyl isosorbide), diethylene glycol monoethyl ether, ethylene glycol monododecyl ether (dodecylglycol), adipic acid diisopropyl ester (diisopropyl adipate), and diethyl sebacate (diethyl decanedioate) are amphiphilic solvents useful for amplifying the penetration of both fat- and water-soluble actives across or into the skin.

Further enhancements for getting actives into the skin can be done with agents capable of complementing the above-mentioned permeation enhancers. Terpenes, plant-derived unsaturated hydrocarbons with the general formula (C₅H₈)ₙ represent a potentially desirable accompaniment in such regard and are often classified as generally regarded as safe (GRAS) by the U.S. Food and Drug Administration. Terpenes can interact with the stratum corneum in multiple ways, but interactions with intercellular lipids by terpenes can move substances through the skin. For example, ceramides, the waxy lipid molecules that form major networks within the stratum corneum, are held tightly together via hydrogen bonds. This connectedness may be loosened or compromised by terpenes with functional groups capable of contributing or accepting hydrogen bonds. Applying terpenes as sorption promoters in concert with ether- and ester-based permeation enhancers can facilitate the entry and traversal of cannabinoids and fatty acid amides across the skin.

In one embodiment, a topical composition can comprise a therapeutically effective amount of at least one cannabinoid receptor modulator. In another aspect, the topical composition can comprise an amount of a combination of a first and second penetration enhancer at synergistic ratios that facilitate a greater combined therapeutic effect than provided by a sum of individual therapeutic effects of the individual components of the combination. In another aspect, the topical composition can comprise a pharmaceutically acceptable carrier suitable for topical administration.

In one aspect, the first penetration enhancer, the second penetration enhancer, or both can be amphiphilic. In another aspect, the first penetration enhancer, the second penetration enhancer, or both can comprise at least one terpene. In another aspect, the at least one terpene can be a cannabis extract. In another aspect, the first penetration enhancer, the second penetration enhancer, or both can comprise one or more of a hexadecenoic acid, a salt of hexadecenoic acid, an ester of hexadecenoic acid, or a combination thereof. In another aspect, the topical composition can comprise a secondary active agent.

In another embodiment, a method of treating a condition in a subject that is responsive to treatment with a cannabinoid receptor modulator can comprise administering a therapeutically effective amount of the topical composition to the subject.

In yet another embodiment, a method of enhancing transdermal penetration of a cannabinoid receptor modulator can comprise: combining an amount of the cannabinoid receptor modulator with a combination of first and second penetration enhancers at synergistic ratios that facilitate a greater combined therapeutic effect than provided by a sum of individual therapeutic effects of the individual components of the combination.

### Compositions

Cannabidiol (CBD) has been under growing study in academic, medical, and commercial settings regarding its potential to treat many clinical indications. The therapeutic potential of the present compositions and methods appears considerable. As a result thereof, a smaller amount of CBD may be used to achieve a therapeutic effect, which could portend reductions in side effects like drug-drug interactions. The present compositions and methods not only have a considerable capacity to enhance transdermal penetration of cannabinoids, but may also be able to do this in short order.

The present compositions or formulations can include a cannabinoid receptor modulator, such as CBD and an amount of a combination of first and second penetration enhancers at synergistic ratios that facilitate a greater combined therapeutic effect than provided by a sum of individual therapeutic effects of the individual components of the combination. In one embodiment, the cannabinoid and the combination of first and second penetration enhancers can be present in a carrier that is suitable for topical administration.

The cannabinoid receptor modulator can be any suitable composition that can be an agonist, an antagonist, an allosteric modulator (positive allosteric modulation, negative allosteric modulation, or neutral allosteric modulation), or the like with respect to a cannabinoid receptor type 1, cannabinoid receptor type 2, or the like. In some embodiments, the cannabinoid receptor modulator can comprise a cannabinoid, a fatty acid amide, or a combination thereof. In one aspect, the cannabinoid can be a phytocannabinoid extract. In another aspect, the cannabinoid can be a synthetic cannabinoid. In yet another aspect, the cannabinoid can be an endocannabinoid.

In some embodiments, the cannabinoid receptor modulator can be a cannabinoid. In particular, the cannabinoid can be any suitable phytocannabinoid extract that can modulate a cannabinoid receptor type 1, a cannabinoid receptor type 2, or the like. In some examples, the phytocannabinoid extract can be extracted from a Cannabis plant. In one aspect, the Cannabis plant can be one or more of: Cannabis sativa, Cannabis indicia, or Cannabis ruderali. In another aspect, the phytocannabinoid extract can be extracted from one or more of: Acmella oleracea, Camellia sinensis, Echinacea purpurea, Echinacea angustifolia, Helichrysum umbraculigerum, Piper methysticum, Radula boninensis, Radula carringtonii, Radula cavifolia, Radula complanate, Radula deflexilobula, Radula demissa, Radula javanica, Radula jonesii, Radula kojana, Radula laxiramea, Radula marginata, Radula obtusiloba, Radula perrottetii, Radida visianica, the like, or a combination thereof. The biochemical composition of Radula species yields physiological effects like those that come from Cannabis extracts.

In one example, the phytocannabinoid extract can comprise, but is not limited to, one or more of a cannabidiol (CBD) component, a tetrahydrocannabinol (THC) component, a cannabigerol (CBG) component, a cannabinol (CBN) component, a cannabichromene (CBC) component, a cannabielsoin (CBE) component, a cannabifuran (CBF) component, a cannabicyclol (CBL) component, a cannabitriol (CBT) component, a cannabinodivarin (CBV) component, a cannabiripsol (CBR) component, a cannabicitran (CBT-C) component, a cannabiglendol-C3 component, the like, or combinations thereof.

When the phytocannabinoid extract comprises a CBD component, the phytocannabinoid extract can comprise, but is not limited to, one or more of: CBD, cannabidiolic acid (CBDA), cannabidiorcol (CBDC1), cannabidiol monomethylether (CBDM), cannabidivarin (CBDV), cannabidivarinic acid (CBDVA), the like, or combinations thereof.

In some aspects, the phytocannabinoid extract can comprise a THC component. In other embodiments, the phytocannabinoid extract does not have a psychoactive component (e.g., a THC component). When the phytocannabinoid extract comprises a THC component, the THC component can comprise, but is not limited to, one or more of: THC, 10-oxo-delta-6a-tetrahydrocannabinol (OTC), delta-8-tetrahydrocannabinolic acid (Δ8-THCA), delta-8-tetrahydrocannabinol (d8THC, Δ8-THC), delta-9-tetrahydrocannabinol (d9THC, Δ9-THC), delta-9-tetrahydrocannabinol-C4 (THC-C4), delta-9-tetrahydrocannabinolic acid A (Δ9-THCA, THCA-A), delta-9-tetrahydrocannabinolic acid B (Δ9-THCB, THCA-B), delta-9-tetrahydrocannabiorolic acid (Δ9-THCA-C1, TCA-C1), delta-9-tetrahydrocannabinolic acid C4 (Δ9-THC-C4, THCA-C4), delta-9-tetrahydrocannabiorcol (Δ9-THC-C1), delta-9-tetrahydrocannabivarin (THCV), delta-9-tetrahydrocannabivarinic acid (THCVA), trihydroxy-delta-9-tetrahydrocannabinol (TRIOH-THC), the like, or combinations thereof.

In other aspects, the phytocannabinoid extract can comprise a CBG component. In this example, the CBG component can comprise, but is not limited to, one or more of CBG, cannabigerolic acid (CBGA), cannabigerolic acid monomethylether (CBGAM), cannabigerovarin (CBGV), cannabigerovarinic acid (CBGVA), cannabigerol monomethylether (CBGM), the like, or combinations thereof.

In some aspects, the phytocannabinoid extract can comprise a CBN component. In this example, the CBN component can comprise, but is not limited to, one or more of: CBN, cannabinoid acid (CBNA), cannabidiorcol (CBN-C1), cannabinol-C2 (CBN-C2), cannabinol-C4 (CBN-C4), cannabinodiol (CBND), cannabinol methylether (CBNM), delta-9-cis-tetrahydrocannabinol (CIS-THC), the like, or combinations thereof.

In other aspects, the phytocannabinoid extract can comprise a CBC component. In one example, the CBC component can comprise, but is not limited to, one or more of: CBC, cannabichromenic acid (CBCA), cannabichromanon (CBCN), cannabichromenevarin (CBCV), cannabichromevarinic acid (CBCVA), the like, or combinations thereof.

In some aspects, the phytocannabinoid extract can comprise a CBE component. In this example, the CBE component can comprise, but is not limited to, one or more of: CBE, cannabielsoic acid A (CBEA-A), cannabielsoic acid B (CBEA-B), the like, or combinations thereof.

In other aspects, the phytocannabinoid extract can comprise a CBF component. In one example, the CBF component can comprise, but is not limited to, one or more of: CBF, dehydrocannabifuran (CBFD), the like, or combinations thereof.

In some aspects, the phytocannabinoid extract can comprise a CBL component. In this example, the CBL component can comprise, but is not limited to, one or more of CBL, cannabicyclolic acid (CBLA), cannabicyclovarin (CBLV), the like, or combinations thereof.

In other aspects, the phytocannabinoid extract can comprise a CBT component. In one example, the CBT component can comprise, but is not limited to, one or more of CBT, cannabitriolvarin (CBTV), 10-Ethoxy-9-hydroxy-delta-6a-tetrahydrocannabinol, 8,9-Dihydroxy-delta-6a-tetrahydrocannabinol, the like, or combinations thereof.

In some aspects, the phytocannabinoid extract can comprise a CBV component. In one example, the CBV component can comprise, but is not limited to, one or more of CBV, cannabinodivarin (CBVD), the like, or combinations thereof. In another aspect,

In other aspects, the phytocannabinoid extract can comprise: (i) a CBR component that comprises, but is not limited to, CBR, or (ii) a CBT-C component that comprises, but is not limited to, CBT-C, or (iii) a cannabiglendol-C3 component that comprises, but is not limited to, cannabiglendol-C3.

In some embodiments, the cannabinoid receptor modulator can be a cannabinoid that is a synthetic cannabinoid. The synthetic cannabinoid can comprise, but is not limited to, one or more of an adamantoylindole, a benzimidazole, a benzoylindole, a cyclohexylphenol, a dibenzopyran, a dibenzopyran hybrid, an eicosanoid, a hydrocarbon, an indazole carboxamide, an indazole-3-carboxamide, an indole-3-carboxamide, an indole-3-carboxylate ester, a naphthoylindazole, a naphthoylindole, a naphthoylpyrrole, a naphthylmethylindene, a naphthylmethylindole, phenylacetylindole, a pyrazolecarboxamide, a tetramethylcyclopropylcarbonylindazole, a tetramethylcyclopropanovlindole, a quinolinyl ester, the like, or combinations thereof.

In one aspect, the synthetic cannabinoid can be an adamantoylindole. In one aspect, the adamantoylindole can be a 3-(1-adamantoyl)indole structure with substitution at the nitrogen atom of the indole ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the adamantoylindole can comprise, but is not limited to, AB-001.

In another aspect, the synthetic cannabinoid can be a benzimidazole. In one example, the benzimidazole can comprise, but is not limited to, one or more of: AZ-11713908, AZD-1940, the like, or a combination thereof.

In one aspect, the synthetic cannabinoid can be a benzoylindole. In one aspect, the benzoylindole can be a 3-(benzoyl)indole structure with substitution at the nitrogen atom of the indole ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the benzoylindole can comprise, but is not limited to, RCS-4.

In another aspect, the synthetic cannabinoid can be a cyclohexylphenol. In one aspect, the cyclohexylphenol can be a 2-(3-hydroxycyclohexyl)phenol structure with substitution at the 5-position of the phenolic ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the cyclohexylphenol can comprise, but is not limited to, one or more of CP 47,497, CP-55,940, the like, or a combination thereof.

In one aspect, the synthetic cannabinoid can be a dibenzopyran. In one example, the dibenzopyran can comprise, but is not limited to, one or more of: AM-087, JWH-051, JWH-056. JWH-057. JWH-065, JWH-091. JWH-102, JWH-103, JWH-124, JWH-130, JWH-133, JWH-138, JWH-139, JWH-142, JWH-143, JWH-186, JWH-187, JWH-188, JWH-190, JWH-191, JWH-215, JWH-216, JWH-217, JWH-224, JWH-225, JWH-226, JWH-227, JWH-229, JWH-230, JWH-233, JWH-247, JWH-254, JWH-256, JWH-277, JWH-278, JWH-298, JWH-299, JWH-300, JWH-301, JWH-310, JWH-336, JWH-338, JWH-339, JWH-340, JWH-341, JWH-349, JWH-350, JWH-351, JWH-352, JWH-353, JWH-354, JWH-355, JWH-356, JWH-357, JWH-358, JWH-359, JWH-360, JWH-361, JWH-362, the like, or combinations thereof. In another aspect, the synthetic cannabinoid can be a dibenzopyran hybrid. In one example, the dibenzopyran hybrid can comprise, but is not limited to, JWH-161.

In one aspect, the synthetic cannabinoid can be an eicosanoid. In one example, the eicosanoid can comprise, but is not limited to, one or more of: AM-883, AM-1346, O-585, O-689, the like, or combinations thereof.

In another aspect, the synthetic cannabinoid can be a hydrocarbon. In one example, the hydrocarbon can comprise, but is not limited to, one or more of: JWH-171, JWH-220, the like, or a combination thereof.

In one aspect, the synthetic cannabinoid can be an indazole carboxamide. In one example, the indazole carboxamide can comprise, but is not limited to, one or more of: AB-INACA, AB-FUBINACA, the like, or a combination thereof.

In another aspect, the synthetic cannabinoid can be an indazole-3-carboxamide. In one aspect, the indazole-3-carboxamide can be a 1H-indazole-3-carboxamide structure with substitution at the nitrogen of the carboxamide by a naphthyl, quinolinyl, isoquinolinyl, adamantyl, or 1-amino-1-oxoalkan-2-yl group and substitution at the one (1) position of the indazole ring by an alkyl, haloalkyl, alkenyl, cycloalkylmethyl, cycloalkylethyl, benzyl, N-methyl-2-piperidinylmethyl, or 2-(4-morpholinyl)ethyl group. In some examples, the indazole-3-carboxamide can comprise, but is not limited to, one or more of: AB-CHMINACA, AB-FUBINACA, PX-2, PX-3, the like, or a combination thereof.

In one aspect, the synthetic cannabinoid can be an indole-3-carboxamide. In one example, the indole-3-carboxamide can comprise, but is not limited to, one or more of CUMYL-BICA, CUMYL-CBMICA, Org 28312, Org 28611, the like, or a combination thereof.

In another aspect, the synthetic cannabinoid can be an indole-3-carboxylate ester. In one example, the indole-3-carboxylate ester can comprise, but is not limited to, one or more of: FDU-PB-22, FBU-PB-22, the like, or a combination thereof.

In one aspect, the synthetic cannabinoid can be a naphthoylindazole. In one example, the naphthoylindazole can comprise, but is not limited to, one or more of: THJ-018, THJ-2201, the like, or a combination thereof.

In another aspect, the synthetic cannabinoid can be a naphthoylindole. In one aspect, the naphthoylindole can be a 3-(1-naphthoyl)indole structure with substitution at the nitrogen atom of the indole ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the naphthoylindole can comprise, but is not limited to, one or more of: AM-1221, AM-2201, JWH-004, JWH-007, JWH-009, JWH-011, JWH-015, JWH-016, JWH-018, JWH-019, JWH-020, JWH-031, JWH-042, JWH-043, JWH-046, JWH-047, JWH-048, JWH-049, JWH-050, JWH-070, JWH-071, JWH-072, JWH-073, JWH-076, JWH-077, JWH-078, JWH-079, JWH-080, JWH-081, JWH-082, JWH-083, JWH-093. JWH-094, JWH-095, JWH-096, JWH-097, JWH-098, JWH-099, JWH-100, JWH-116, JWH-120, JWH-122, JWH-148, JWH-149, JWH-151, JWH-153, JWH-159, JWH-160, JWH-163, JWH-164, JWH-165, JWH-166, JWH-180, JWH-181, JWH-182, JWH-189, JWH-193, JWH-198, JWH-200, JWH-210, JWH-211, JWH-212, JWH-213, JWH-234, JWH-235, JWH-236, JWH-239, JWH-240, JWH-241, JWH-242, JWH-258, JWH-259, JWH-260, JWH-261, JWH-262, JWH-265, JWH-266, JWH-267, JWH-268, JWH-387, JWH-398, JWH-416, JWH-417, JWH-422, JWH-423, JWH-424, JWH-425, WIN-55,212-2, the like, or combinations thereof.

In one aspect, the synthetic cannabinoid can be a naphthoylpyrrole. In one aspect, the naphthoylpyrrole can be a 3-(1-naphthoyl)pyrrole structure with substitution at the nitrogen atom of the pyrrole ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the naphthoylpyrrole can comprise, but is not limited to, one or more of: JWH-030, JWH-032, JWH-033, JWH-036, JWH-044, JWH-045, JWH-145, JWH-146, JWH-147, JWH-150, JWH-156, JWH-243, JWH-244, JWH-245, JWH-246, JWH-292, JWH-293, JWH-307, JWH-308, JWH-309, JWH-346, JWH-347, JWH-348, JWH-364, JWH-365, JWH-366, JWH-367, JWH-368, JWH-369, JWH-370, JWH-371, JWH-372, JWH-373, the like, or combinations thereof.

In another aspect, the synthetic cannabinoid can be a naphthylmethylindene. In one aspect, the naphthylmethylindene can be a 1-(1-naphthylmethyl)indene structure with substitution at the 3-position of the indene ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the naphthylmethylindene can comprise, but is not limited to, JWH-176.

In one aspect, the synthetic cannabinoid can be a naphthylmethylindole. In one aspect, the naphthylmethylindole can be a 1H-indol-3-yl-(1-naphthyl)methane structure with substitution at the nitrogen atom of the indole ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the naphthylmethylindole can comprise, but is not limited to, one or more of: JWH-175, JWH-184, JWH-185, JWH-192, JWH-194, JWH-195, JWH-196, JWH-197, JWH-199, the like, or a combination thereof.

In another aspect, the synthetic cannabinoid can be a phenylacetylindole. In one aspect, the phenylacetylindole can be a 3-phenylacetylindole structure with substitution at the nitrogen atom of the indole ring by an alkyl, haloalkyl, alkenyl, cycloalkylmethyl, cycloalkylethyl, 1-(N-methyl-2-piperidinyl)methyl, or 2-(4-morpholinyl)ethyl group. In one example, the phenylacetylindole can comprise, but is not limited to, one or more of: JWH-167, JWH-202, JWH-203, JWH-204, JWH-205, JWH-206, JWH-207, JWH-208, JWH-209, JWH-237, JWH-248, JWH-249, JWH-250, JWH-251, JWH-252, JWH-253, JWH-303, JWH-304, JWH-305, JWH-306, JWH-311, JWH-312, JWH-313, JWH-314, JWH-315, JWH-316, RCS-8, the like, or combinations thereof.

In one aspect, the synthetic cannabinoid can be a pyrazolecarboxamide. In one example, the pyrazolecarboxamide can comprise, but is not limited to, one or more of 5F-AB-FUPPYCA, AB-CHFUPYCA, the like, or a combination thereof.

In another aspect, the synthetic cannabinoid can be a tetramethylcyclo-propylcarbonylindazole. In one example, the tetramethylcyclo-propylcarbonylindazole can comprise, but is not limited to, FAB-144.

In one aspect, the synthetic cannabinoid can be a tetramethylcyclo-propanoylindole. In one aspect, the tetramethylcyclo-propanoylindole can be a 3-(1-tetramethylcyclopropoyl)indole structure with substitution at the nitrogen atom of the indole ring by an alkyl, haloalkyl, cycloalkylmethyl, cycloalkylethyl, 1-(N-methyl-2-piperidinyl)methyl, or 2-(4-morpholinyl)ethyl group. In one example, the tetramethylcyclo-propanoylindole can comprise, but is not limited to, XLR-11.

In another aspect, the synthetic cannabinoid can be a quinolinyl ester. In one example, the quinolinyl ester can comprise, but is not limited to, one or more of: PB-22, 5F-PB-22, the like, or a combination thereof.

In another aspect, the synthetic cannabinoid can comprise, but is not limited to, one or more of: AM-251, AM-279, AM-281, AM-356, AM-374, AM-381, AM-404, AM-411, AM-630, AM-661, AM-678, AM-679, AM-694, AM-735, AM-855, AM-881, AM-905, AM-906, AM-919, AM-926, AM-938, AM-1116, AM-1172, AM-1220, AM-1235, AM-1241, AM-1248, AM-1710, AM-1714, AM-1902, AM-2212, AM-2213, AM-2232, AM-2233, AM-2389, AM-3102, AM-4030, AM-4054, AM-4056, AM-4113, AM-6545, AM-7438, CP-50,566-1, CP-55,244, CP-945-598, HU-210, HU-211, HU-239, HU-243, HU-308, HU-320, HU-331, HU-336, HU-345, 5F-AB-PINACA, 5F-AMB, 5F-ADB, 5F, -NNE1, 5F-PCN, 5F-SDB-006, AB-005, ADAMANTYL-THPINACA, AMB-FUBINACA, APICA, APINACA, BB-22, BIM-018, FDU-NNE1, FUB-144, FUBIMINA, MDMB-CHMICA, MDMB-FUBINACA, MMB-2201, MN-18, NM-2201, NNE1, PX1, SDB-005, SDB-006, STS-135, XLR-12, the like, or a combination thereof.

In another aspect, the synthetic cannabinoid can comprise, but is not limited to, one or more of: Nabilone, Rimonabant (SR141716), Nabiximols, Dronabinol, Dimethylheptylpyran, SR144528, the like, or combinations thereof.

In some embodiments, the cannabinoid receptor modulator can be a cannabinoid that is an endocannabinoid. In one example, the endocannabinoid can comprise, but is not limited to, one or more of: anandamide (ANA), 2-arachidonoylglycerol (2-AG), 2-arachidonyl glyceryl ether, N-arachidonoyl dopamine (NADA), virodhamine (OAE), lysophosphatidylinositol (LPI), the like, or combinations thereof.

In some embodiments, the topical composition does not comprise or otherwise excludes a psychoactive cannabinoid. In one example, the topical composition does not comprise, or otherwise excludes THC or any other cannabinoid that has psychoactive properties.

In some embodiments, the cannabinoid receptor modulator can comprise a fatty acid amide. In one aspect, the fatty acid amide can comprise, but is not limited to, one or more of: an N-acylethanolamine, a fatty acid primary amide (FAPA), the like, or a combination thereof. In one example, the N-acylethanolamine can comprise, but is not limited to, one or more of anandamide, N-palmitoylethanolamine, N-oleoylethanolamine, N-stearoylethanolamine, N-docosahexaenoylethanolamine, N-docosatetraenoylethanolamine, N-eicosapentaenoylethanolamide, the like, or combinations thereof. In another example, the FAPA can comprise, but is not limited to, oleamide.

In some embodiments, the cannabinoid receptor modulator can be present in the topical composition at a concentration of from about 0.0001 wt % to about 80 wt %. In one aspect, the cannabinoid receptor modulator can be present in the topical composition at a concentration of from about 0.0001 wt % to about 50 wt %. In another aspect, the cannabinoid receptor modulator can be present in the topical composition at a concentration of from about 0.1 wt % to about 10 wt %. In yet another aspect, the cannabinoid receptor modulator can be present in the topical composition at a concentration of from about 0.1 wt % to about 5 wt %.

In some embodiments, the cannabinoid receptor modulator and the combination of first and second penetration enhancers can be present in a ratio of from about 20:1 to about 1:500. In one aspect, the cannabinoid receptor modulator and the combination of first and second penetration enhancers can be present in a ratio of from about 10:1 to about 1:100. In another aspect, the cannabinoid receptor modulator and the combination of first and second penetration enhancers can be present in a ratio of from about 20:1 to about 1:20. In yet another aspect, the cannabinoid receptor modulator and the combination of first and second penetration enhancers can be present in a ratio of from about 10:1 to about 1:10.

In some embodiments, the first penetration enhancer, the second penetration enhancer m or both can comprise at least one terpene. In one aspect, the terpene can be a cannabis extract. In another aspect, the terpene can be a lipophilic terpene.

In one aspect the terpene can comprise any suitable terpene that is a penetration enhancer. In one example, the terpene can be any suitable terpene as provided in Table A:

TABLE A
Penetration Enhancement Terpenes

| Name | IUPAC NAME | cLogP |
|---|---|---|
| cyclohexanemethanol | cyclohexylmethanol | 1.3946 |
| menthone | 5-methyl-2-(propan-2-yl)cyclohexan-1-one | 2.5549 |
| CE≤-citronellol | (3R)-3,7-dimethyloct-6-en-1-ol | 3.3494 |
| geraniol | (2E)-3,7-dimethylocta-2,6-dien-1-ol | 3.4853 |
| nerol | (2Z)-3,7-dimethylocta-2,6-dien-1-ol | 3.4853 |
| squalene | (6E,10E,14E,18E)-2,6,10,15,19,23-hexamethyltetracosa- | 13.099 |
| | 2,6,10,14,18,22-hexaene | |
| (−)-isolongifolol | [(1R,2S,7R,8S,9S)-3,3,7- | 3.1597 |
| | trimethyltricyclo[5.4.0.0¬≤,,Åπ]undecan-8-yl]methanol | |
| (−)-caryophyllene | (1R,4R,6R,10S)-4,12,12-trimethyl-9-methylidene-5- | 4.0553 |
| oxide | oxatricyclo[8.2.0.0,Å¥,,Å∂]dodecane | |
| octisalate | 2-ethylhexyl 2-hydroxybenzoate | 4.1235 |
| Fenchone | 1,3,3-trimethylbicyclo[2.2.1]heptan-2-one | 2.1793 |
| Limonene oxide | 1-methyl-4-(prop-1-en-2-yl)-7-oxabicyclo[4.1.0]heptane | 2.3448 |
| myrcene | 7-methyl-3-methylideneocta-1,6-diene | 4.2932 |
| Pulegone | 5-methyl-2-(propan-2-ylidene)cyclohexan-1-one | 2.8501 |
| Pinene oxide | 2,7,7-trimethyl-3-oxatricyclo[4.1.1.0¬≤,Å¥]octane | 1.7044 |
| (S)-(−)-perillaldehyde | (4S)-4-(prop-1-en-2-yl)cyclohex-1-ene-1-carbaldehyde | 2.2224 |
| (−)-dihydrocarveol | (1R,2R,5R)-2-methyl-5-(prop-1-en-2-yl)cyclohexan-1-ol | 2.6138 |
| (+)-dihydrocarveol | (1S,2S,5S)-2-methyl-5-(prop-1-en-2-yl)cyclohexan-1-ol | 2.6138 |
| alpha Pinene | 2,6,6-trimethylbicyclo[3.1.1]hept-2-ene | 2.721 |
| 7-Oxabicylo-(2-2- | 7-oxabicyclo[2.2.1]heptane | 0.9022 |
| 1)heptane | | |
| Cyclopentene oxide | 6-oxabicyclo[3.1.0]hexane | 0.5602 |
| retinoic acid | (2E,4E,6E,8E)-3,7-dimethyl-9-(2,6,6-trimethylcyclohex-1-en-1- | 5.8207 |
| | yl)nona-2,4,6,8-tetraenoic acid | |
| farnesol | 3,7,11-trimethyldodeca-2,6,10-trien-1-ol | 5.6571 |
| (−)-CE±-cedrene | (1S,2R,5S,7S)-2,6,6,8-tetramethyltricyclo[5.3.1.0¬π,,Åμ]undec- | 3.9819 |
| | 8-ene | |
| EUCALYPTOL | 1,3,3-trimethyl-2-oxabicyclo[2.2.2]octane | 2.1095 |
| (+)-longifolene | (1R,2S,7S,9S)-3,3,7-trimethyl-8- | 4.0602 |
| | methylidenetricyclo[5.4.0.0¬≤,Åπ]undecane | |
| (−)-CE±-santonin | (3S,3aS,5aS,9bS)-3,5a,9-trimethyl- | 1.8467 |
| | 2H,3H,3aH,4H,5H,5aH,8H,9bH-naphtho[1,2-b]furan-2,8-dione | |
| (+)-aromadendrene | (1aR,4aR,7R,7aR,7bS)-1,1,7-trimethyl-4-methylidene- | 3.998 |
| | decahydro-1H-cyclopropa[e]azulene | |
| (−)-guaiol | 2-[(3S,5R,8S)-3,8-dimethyl-1,2,3,4,5,6,7,8-octahydroazulen-5- | 3.6689 |
| | yl]propan-2-ol | |
| carvacrol | 2-methyl-5-(propan-2-yl)phenol | 2.8448 |
| ocimene | (3E)-3,7-dimethylocta-1,3,7-triene | 4.2932 |
| eucarvone | 2,6,6-trimethylcyclohepta-2,4-dien-1-one | 2.1758 |
| Ascaridole | 1-methyl-4-(propan-2-yl)-2,3-dioxabicyclo[2.2.2]oct-5-ene | 2.3961 |
| (1R)-(−)-myrtenol | {6,6-dimethylbicyclo[3.1.1]hept-2-en-2-yl}methanol | 1.7943 |
| (¬±)-CE±-bisabolol | 6-methyl-2-(4-methylcyclohex-3-en-1-yl)hept-5-en-2-ol | 4.4711 |
| citral | (2E)-3,7-dimethylocta-2,6-dienal | 3.273 |
| (+)-CE≤-cedrene | (1S,2R,5S,7S)-2,6,6-trimethyl-8- | 4.0602 |
| | methylidenetricyclo[5.3.1.0¬π,,Åμ]undecane | |
| (−)-CE±-thujone | (1S,4R,5R)-4-methyl-1-(propan-2-yl)bicyclo[3.1.0]hexan-3-one | 2.1785 |
| (+)-dihydrocarvone | (2R,5R)-2-methyl-5-(prop-1-en-2-yl)cyclohexan-1-one | 2.7575 |
| Terpinen-4-ol | 4-methyl-1-(propan-2-yl)cyclohex-3-en-1-ol | 2.3354 |
| (1R)-(−)-myrtenal | 6,6-dimethylbicyclo[3.1.1]hept-2-ene-2-carbaldehyde | 1.582 |
| terpinolene | 1-methyl-4-(propan-2-ylidene)cyclohex-1-ene | 3.454 |
| (S)-(−)-citronellal | (3S)-3,7-dimethyloct-6-enal | 3.1371 |
| d-Limonene | 1-methyl-4-(prop-1-en-2-yl)cyclohex-1-ene | 3.3614 |
| (R)-(−)-carvone | (SR)-2-methyl-5-(prop-1-en-2-yl)cyclohex-2-en-1-one | 2.653 |
| CE±-humulene | 2,6,6,9-tetramethylcycloundeca-1,4,8-triene | 6.2387 |
| retinol | (2E,4E,6E,8E)-3,7-dimethyl-9-(2,6,6-trimethylcyclohex-1-en-1- | 6.1703 |
| | yl)nona-2,4,6,8-tetraen-1-ol | |
| Nerolidol | 3,7,11-trimethyldodeca-1,6,10-trien-3-ol | 5.4029 |
| (¬±)-nerolidol | (6E)-3,7,11-trimethyldodeca-1,6,10-trien-3-ol | 5.4029 |
| CE≤-carotene | 1,3,3-trimethyl-2-[(1E,3E,5E,7E,9E,11E,13E,15E,17E)- | 13.873 |
| | 3,7,12,16-tetramethyl-18-(2,6,6-trimethylcyclohex-1-en-1- | |
| | yl)octadeca-1,3,5,7,9,11,13,15,17-nonaen-1-yl]cyclohex-1-ene | |
| 3-Carene | 3,7,7-trimethylbicyclo[4.1.0]hept-3-ene | 2.721 |
| phytol | (2E,7R,11R)-3,7,11,15-tetramethylhexadec-2-en-1-ol | 7.4212 |
| (+)-cedrol | (1S,2R,5S,7R,8R)-2,6,6,8- | 3.263 |
| | tetramethyltricyclo[5.3.1.0¬π,,Åμ]undecan-8-ol | |
| (+)-cedryl acetate | (1S,2R,5S,7R,8R)-2,6,6,8- | 3.7476 |
| | tetramethyltricyclo[5.3.1.0¬π,,Åμ]undecan-8-yl acetate | |
| (¬±)-linalool | 3,7-dimethylocta-1,6-dien-3-ol | 3.2311 |
| Fennel oil | 1,3,3-trimethylbicyclo[2.2.1]heptan-2-one; 1-methoxy-4-(prop- | 2.1793 |
| | 1-en-1-yl)benzene; 1-methoxy-4-(prop-2-en-1-yl)benzene | |
| Peppermint oil | 3,6-dimethyl-4,5,6,7-tetrahydro-1-benzofuran; 3,7-dimethyl-1- | 2.8958 |
| | oxaspiro[3.5]nonane; 5-methyl-2-(propan-2-yl)cyclohexan-1-ol; | |
| | 5-methyl-2-(propan-2-yl)cyclohexan-1-one; 5-methyl-2- | |
| | (propan-2-yl)cyclohexyl acetate; 5-methyl-2-(propan-2- | |
| | ylidene)cyclohexan-1-one | |
| (−)-trans- | (1R,4E,9S)-4,11,11-trimethyl-8- | 5.4861 |
| caryophyllene | methylidenebicyclo[7.2.0]undec-4-ene | |
| (−)-epiglobulol | (1aR,4S,4aR,7R,7aS,7bS)-1,1,4,7-tetramethyl-decahydro-1H- | 3.2008 |
| | cyclopropa[e]azulen-4-ol | |
| L-(−)-menthol | (1R,2S,SR)-5-methyl-2-(propan-2-yl)cyclohexan-1-ol | 2.4112 |
| (−)-isopulegol | (1R,2S,SR)-5-methyl-2-(prop-1-en-2-y])cyclohexan-1-ol | 2.6138 |
| piperitone | 3-methyl-6-(propan-2-yl)cyclohex-2-en-1-one | 2.4504 |
| (R)-(+)-pulegone | (5R)-5-methyl-2-(propan-2-ylidene)cyclohexan-1-one | 2.8501 |
| thymol | 5-methyl-2-(propan-2-yl)phenol | 2.8448 |
| alpha Terpineol | 2-(4-methylcyclohex-3-en-1-yl)propan-2-ol | 2.2993 |
| (−)-carveol | 2-methyl-5-(prop-1-en-2-yl)cyclohex-2-en-1-ol | 2.5093 |
| Carvone | 2-methyl-5-(prop-1-en-2-yl)cyclohex-2-en-1-one | 2.653 |
| CE±-phellandrene | 2-methyl-5-(propan-2-yl)cyclohexa-1,3-diene | 2.8834 |

When the terpene is a cannabis extract, the terpene can be extracted from a Cannabis plant. In one aspect, the Cannabis plant can be one or more of: Cannabis sativa, Cannabis indicia, or Cannabis ruderali. In one example, the terpenes that can be extracted from a Cannabis plant can comprise, but are not limited to, one or more of: myrcene, beta-caryophyllene, limonene, linalool, pinene, humulene, terpinolene, alpha-Bisabolol, eucalyptol, geraniol, terpineol, farnesene, borneol, ocimene, nerolidol, guaiol, valencene, delta-3 Carene, phytol, sabinene, phellandrene, fenchol, menthol, terpinene, isoborneol, cymene, the like, or a combination thereof.

In some examples, the terpene can be extracted or otherwise derived or provided from non-Cannabis plant sources. In one aspect, Bisabolol can be extracted from one or more of: Chamomile (Matricaria chamomilla) Oil, Candeia Tree (Eremanthus erythropappus) Oil, the like, or combinations thereof.

In another aspect, plant species that contain Myrcene can be: Wild Thyme (Thymus serpyllum) Oil, (Verbenaceae Verbena species), Hairy Chinaflower (Adenandra villosa) Oil, Dill (Anethum graveolens), Bay leaf (Laurus nobilis) Oil, Mango (Mangifera indica), Cannabis (Cannabis sativa, C. indica, C, ruderalis) Oil, Hops (Humulus lupulus) Oil, Citronella (Cymbopogon nardus) Oil, Lemongrass (Cymbopogon citratus) Oil, the like, or combinations thereof.

In another aspect, beta-caryophyllene can be extracted from one or more of: Copaiba (Leguminosae Copaiba species) Oil, Caraway (Apiaceae Carum species) Oil, Basil (Ocimum basilicum) Oil, Oregano (Origanum vulgare) Oil, West African Pepper Families (Annonaceae, Piperaceae and Zingiberaceae; also Species Xylopia aethiopica) Oil, Rosemary (Rosmarinus officinalis) Oil, Cinnamon (Lauraceae Cinnamomum species) Oil, Ylang-Ylang (Cananga odorata) Oil, the like, or combinations thereof.

In another aspect, limonene can be extracted from one or more of: Wild Orange (Capparis mitchellii) Oil, Bergamot (Monarda fistulosa) Oil, Lemon (Citrus limon) Oil, Grapefruit (Citrus sinensis X Citrus maxima) Oil, Lime Key lime (Citrus aurantifolia and species of citrus trees whose fruits are limes) Oil, Bitter Orange (Citrus aurantium) Oil, Elemi (Canarium luzonicum) Oil, Pomelo (Citrus grandis) Oil, Esrog Oil (Citrus medica), Mandarin Orange (Citrus reticulata) Oil, Trifoliate Orange (Poncirus trifoliata) Oil, the like, or combinations thereof.

In another aspect, linalool can be extracted from one or more of: Coriander (Coriandrum sativum) Oil, Basil (Ocimum basilicum) Oil, Lavender (Lamiaceae Lavandula species) Oil, Bergamot (Monarda fistulosa) Oil, Sage (Labiatae Salvia species) Oil, Cinnamon (Lauraceae Cinnamomum species) Oil, Ylang-Ylang (Cananga odorata) Oil, Geranium (Geraniaceae Geranium species) Oil, the like, or combinations thereof.

In another aspect, myrcene can be extracted from one or more of: Wild Thyme (Thymus serpyllum) Oil, (Verbenaceae Verbena species), Hairy Chinaflower (Adenandra villosa) Oil, Dill (Anethum graveolens), Bay leaf (Laurus nobilis) Oil, Mango (Mangifera indica), Cannabis (Cannabis sativa, C. indica, C, ruderalis) Oil, Hops (Humulus lupulus) Oil, Citronella (Cymbopogon nardus) Oil, Lemongrass (Cymbopogon citratus) Oil, the like, or combinations thereof.

In another aspect, terpinolene can be extracted from one or more of: Sage (Salvia officianalis) Oil, Lilac (Oleaceae Syringa species) Oil, Rosemary, (Rosmarinus officinalis) Oil, Conifer Tree (Order Pinales) Oils, Apple Tree (Malus domestica) Oil, Ginger (Zingiber officinale) Oil; Hyssop (Hyssopus officinalis) Oil, Clove (Syzygium aromaticum) Oil, Almond (Prunus amygdalus) Oil, β-Pinene, β-Ocimene, Humulene, the like, or combinations thereof.

In some embodiments, the terpene can be a lipophilic terpene that can have a calculated log P (c Log P) that is selected to increase the penetration enhancement activities of the topical composition when combined with another penetration enhancer. The c Log P can be calculated as the logarithm of the partition coefficient between n-octanol and water log(c_{octanol}/c_{water}) as a measure of lipophilicity.

In one aspect, the terpene can have a c Log P of greater than 0. In one example, the terpene can have a c Log P of greater than 0 when the terpene comprises one or more of: (+)-aromadendrene, (+)-cedrol, (+)-cedryl acetate, (+)-dihydrocarveol, (+)-dihydrocarvone, (+)-longifolene, (+)-β-cedrene, (−)-carveol, (−)-caryophyllene oxide, (−)-dihydrocarveol, (−)-epiglobulol, (−)-guaiol, (−)-isolongifolol, (−)-isopulegol, (−)-trans-caryophyllene, (−)-α-cedrene, (−)-α-santonin, (−)-α-thujone, (1R)-(−)-myrtenal, (1R)-(−)-myrtenol, (R)-(+)-pulegone, (R)-(−)-carvone, (S)-(−)-citronellal, (S)-(−)-perillaldehyde, (±)-linalool, (±)-nerolidol, (±)-α-bisabolol, 3-Carene, 7-Oxabicylo-(2-2-1)heptane, alpha Pinene, alpha Terpineol, Ascaridole, carvacrol, Carvone, citral, cyclohexanemethanol, Cyclopentene oxide, d-Limonene, EUCALYPTOL, eucarvone, farnesol, Fenchone, Fennel oil, geraniol, L-(−)-menthol, Limonene oxide, menthone, myrcene, nerol, Nerolidol, ocimene, octisalate, Peppermint oil, phytol, Pinene oxide, piperitone, Pulegone, retinoic acid, retinol, squalene, Terpinen-4-ol, terpinolene, thymol, α-humulene, α-phellandrene, β-carotene, β-citronellol, the like, or combinations thereof.

In another aspect, the terpene can have a c Log P of greater than 2.0. In one example, the terpene can have a c Log P of greater than 2.0 when the terpene comprises one or more of (+)-aromadendrene, (+)-cedrol, (+)-cedryl acetate, (+)-dihydrocarveol, (+)-dihydrocarvone, (+)-longifolene, (+)-β-cedrene, (−)-carveol, (−)-caryophyllene oxide, (−)-dihydrocarveol, (−)-epiglobulol, (−)-guaiol, (−)-isolongifolol, (−)-isopulegol, (−)-trans-caryophyllene, (−)-α-cedrene, (−)-α-thujone, (R)-(+)-pulegone, (R)-(−)-carvone, (S)-(−)-citronellal, (S)-(−)-perillaldehyde, (±)-linalool, (±)-nerolidol, (±)-α-bisabolol, 3-Carene, alpha Pinene, alpha Terpineol, Ascaridole, carvacrol, Carvone, citral, d-Limonene, EUCALYPTOL, eucarvone, farnesol, Fenchone, Fennel oil, geraniol, L-(−)-menthol, Limonene oxide, menthone, myrcene, nerol, Nerolidol, ocimene, octisalate, Peppermint oil, phytol, piperitone, Pulegone, retinoic acid, retinol, squalene, Terpinen-4-ol, terpinolene, thymol, α-humulene, α-phellandrene, β-carotene, β-citronellol, the like, or combinations thereof.

In another aspect, the terpene can have a c Log P of greater than 3.0. In one example, the terpene can have a c Log P of greater than 3.0 when the terpene comprises one or more of (+)-aromadendrene, (+)-cedrol, (+)-cedryl acetate, (+)-longifolene, (+)-β-cedrene, (−)-caryophyllene oxide, (−)-epiglobulol, (−)-guaiol, (−)-isolongifolol, (−)-trans-caryophyllene, (−)-α-cedrene, (S)-(−)-citronellal, (±)-linalool, (±)-nerolidol, (±)-α-bisabolol, citral, d-Limonene, farnesol, geraniol, myrcene, nerol, Nerolidol, phytol, retinoic acid, retinol, squalene, terpinolene, α-humulene, β-carotene, β-citronellol, the like, or combinations thereof.

In another aspect, the terpene can have a suitable penetration enhancement activity when combined with another penetration enhancer when the terpene comprises, but is not limited to, one or more of: biasbolol, β-caryophyllene, D-limonene, linalool, nerolidol, myrcene, terpinolene, the like, or combinations thereof.

In another aspect, the terpene can have a suitable penetration enhancement activity when combined with another penetration enhancer when the terpene comprises, but is not limited to, one or more of: terpinolene, D-limonene, (−)-trans-caryophyllene, the like, or combinations thereof.

In one aspect, the cannabinoid receptor modulator and the terpene can be present in a ratio of from about 10:1 to about 1:100. In another aspect, the first penetration enhancer and the terpene can be present in a ratio of from about 5:1 to about 1:50.

In other embodiments, the first penetration enhancer, the second penetration enhancer, or both can be amphiphilic. In one aspect, the first penetration enhancer, the second penetration enhancer, or both can have a c Log P of greater than one or more of: 0, 1, 2, 3, 5, or a combination thereof. In another aspect, the first penetration enhancer, the second penetration enhancer, or both can have a polar surface area of greater than one or more of: 20, 35, 50, or a combination thereof. The polar surface area can be the topological surface area. In another aspect, the first penetration enhancer, the second penetration enhancer, or both can have molecular weight that is selected to enhance penetration enhancement. In one aspect, the molecular weight can be less than one or more of: 1000, 500, 400, 300, 200, 100, or combinations thereof.

In other embodiments, the first penetration enhancer, the second penetration enhancer, or both comprise one or more of: lower chain (C2 to C4) alcohols (see e.g., Table B), lower chain diols (such as propylene glycol, di-propylene glycol) (see e.g., Table C), triacetin, glycerol monooleate, glycerol monolaurate, oleic alcohol, lauryl alcohol, isopropyl myrisate, sorbitan esters (see e.g., Table D), ethers (see e.g., Table E), other surfactant enhancers (see e.g., Table F), the like, or combinations thereof.

In one aspect, the first penetration enhancer, the second penetration enhancer, or both can be any suitable alcohol as shown in Table B:

TABLE B
Alcohol Penetration Enhancers

| | | | Polar |
|---|---|---|---|
| | Molecular | | Surface |
| Name | Weight | cLogP | Area |
| non-3-en-1-ol | 142.241 | 2.9791 | 20.23 |
| hexan-1-1 | 102.176 | 1.8681 | 20.23 |
| octan-1-ol | 130.23 | 2.7769 | 20.23 |
| decan-1-o1 | 158.284 | 3.6857 | 20.23 |
| undecan-1-o1 | 172.311 | 4.1401 | 20.23 |
| dodecan-1-ol | 186.337 | 4.5945 | 20.23 |
| tetradecan-1-ol | 214.391 | 5.5033 | 20.23 |
| octadecan-1-o1 | 270.499 | 7.3209 | 20.23 |
| octan-2-ol | 130.23 | 2.6818 | 20.23 |
| nonan-1-ol | 144.257 | 3.2313 | 20.23 |
| octadec-9-en-1-ol | 268.483 | 7.0687 | 20.23 |
| oct-3-en-1-o1 | 128.214 | 2.5247 | 20.23 |
| 14-methylpentadecan-1-ol | 242.445 | 6.176 | 20.23 |
| 16-methylheptadecan-1-ol | 270.499 | 7.0848 | 20.23 |
| hexadecan-1-ol | 242.445 | 6.4121 | 20.23 |
| octadeca-9,12-dien-1-ol | 266.467 | 6.8165 | 20.23 |
| octadeca-9,12,15-trien-1-ol | 264.451 | 6.5643 | 20.23 |
| heptan-1-ol | 116.203 | 2.3225 | 20.23 |
| heptan-2-o1 | 116.203 | 2.2274 | 20.23 |
| hex-3-en-1-o1 | 100.16 | 1.6159 | 20.23 |
| heptan-4-ol | 116.203 | 2.2274 | 20.23 |
| octan-4-ol | 130.23 | 2.6818 | 20.23 |
| heptan-3-ol | 116.203 | 2.2274 | 20.23 |
| octan-3-ol | 130.23 | 2.6818 | 20.23 |
| nonan-4-ol | 144.257 | 3.1362 | 20.23 |
| hexan-3-ol | 102.176 | 1.773 | 20.23 |
| nonan-5-ol | 144.257 | 3.1362 | 20.23 |
| nonan-3-ol | 144.257 | 3.1362 | 20.23 |
| hexan-2-o1 | 102.176 | 1.773 | 20.23 |
| nonan-2-ol | 144.257 | 3.1362 | 20.23 |
| ethanol | 46.0686 | 0.0505 | 20.23 |
| methanol | 32.0418 | −0.3558 | 20.23 |
| propan-2-o1 | 60.0955 | 0.4098 | 20.23 |
| propan-1-o1 | 60.0955 | 0.5049 | 20.23 |
| butan-1-ol | 74.1224 | 0.9593 | 20.23 |
| pentan-1-ol | 88.1493 | 1.4137 | 20.23 |
| pent-3-en-1-ol | 86.1334 | 1.1615 | 20.23 |

In another aspect, the first penetration enhancer, the second penetration enhancer, or both can be any suitable polyol as shown in Table C:

TABLE C
Polyol Penetration Enhancers

| | | | Polar |
|---|---|---|---|
| | Molecular | | Surface |
| Name | Weight | cLogP | Area |
| nonane-1,2,3-triol | 176.255 | 1.1877 | 60.69 |
| 2-(2-hydroxypropoxy)propan-1-ol | 134.174 | −0.2501 | 49.69 |
| ethane-1,2-diol | 62.0676 | −0.8762 | 40.46 |
| octane-1,2-diol | 146.229 | 1.7551 | 40.46 |
| (6-{[3,4-dihydroxy-2,5-bis(hydroxymethyl)oxolan-2- | 580.709 | 2.236 | 195.6 |
| yl]oxy}-3,4,5-trihydroxyoxan-2-yl)methyl hexadecanoate | | | |
| (6-{[3,4-dihydroxy-2,5-bis(hydroxymethyl)oxolan-2- | 606.747 | 2.8926 | 195.6 |
| yl]oxy}-3,4,5-trihydroxyoxan-2-yl)methyl octadec-9- | | | |
| enoate | | | |
| [3,4-dihydroxy-5-(hydroxymethyl)-2-{[3,4,5-trihydroxy-6- | 552.655 | 1.3272 | 195.6 |
| (hydroxymethyl)oxan-2-yl]oxy}oxolan-2-yl]methyl | | | |
| tetradecanoate | | | |
| [5-({6-[(dodecanoyloxy)methyl]-3,4,5-trihydroxyoxan-2- | 706.907 | 5.447 | 201.67 |
| yl}oxy)-3,4-dihydroxy-5-(hydroxymethyl)oxolan-2- | | | |
| y]methyl dodecanoate | | | |
| 2-[3,4-dihydroxy-2,5-bis(hydroxymethyl)oxolan-2-yl]- | 524.601 | −0.3693 | 206.6 |
| 3,4,5-trihydroxy-6-(hydroxymethyl)oxan-2-yl dodecanoate | | | |
| [3,4-dihydroxy-5-(hydroxymethyl)-5-{[3,4,5-trihydroxy-6- | 606.747 | 2.8926 | 195.6 |
| (hydroxymethyl)oxan-2-yl]oxy}oxolan-2-yl]methyl | | | |
| octadec-9-enoate | | | |
| 2-(hydroxymethyl)-6-(octyloxy)oxane-3,4,5-triol | 292.37 | 0.9398 | 99.38 |
| (5-[(dodecanoyloxy)methyl]-3,4-dihydroxy-2-{[3,4,5- | 706.907 | 5.447 | 201.67 |
| trihydroxy-6-(hydroxymethyl)oxan-2-yl]oxy}oxolan-2- | | | |
| yl}methyl dodecanoate | | | |
| 2-[(6-{[3,4-dihydroxy-2,5-bis(hydroxymethyl)oxolan-2- | 504.436 | −6.4473 | 268.68 |
| yl]oxy}-3,4,5-trihydroxyoxan-2-yl)methoxy]-6- | | | |
| (hydroxymethyl)oxane-3,4,5-triol | | | |
| butane-2,3-diol | 90.1214 | −0.1576 | 40.46 |
| 2-{[3,4-dihydroxy-2,5-bis(hydroxymethyl)oxolan-2- | 342.296 | −4.6102 | 189.53 |
| yl]oxy}-6-(hydroxymethyl)oxane-3,4,5-triol | | | |
| propane-1,2-diol | 76.0945 | −0.5169 | 40.46 |
| 2-(decyloxy)-6-(hydroxymethyl)oxane-3,4,5-triol | 320.424 | 1.8486 | 99.38 |
| 2-(dodecyloxy)-6-(hydroxymethyl)oxane-3,4,5-triol | 348.478 | 2.7574 | 99.38 |
| oxirane; propane-1,2,3-triol | 136.146 | −1.4436 | 60.69 |
| hexane-1,2,3,4,5,6-hexol | 182.171 | −3.1458 | 121.38 |
| hexane-1,2-diol | 118.175 | 0.8463 | 40.46 |
| 2-(hydroxymethyl)-6-(octylsulfany])oxane-3,4,5-triol | 308.437 | 1.9498 | 115.45 |
| sodium 3-{[3-acetamido-5-hydroxy-6- | 403.315 | −6.021 | 198.07 |
| (hydroxymethyl)oxan-2-yl]oxy}-4,5,6-trihydroxyoxane-2- | | | |
| carboxylate | | | |
| 2-[3,4-bis(2-hydroxyethoxy)oxolan-2-yl]-2-hydroxyethyl | 434.567 | 2.516 | 114.68 |
| dodecanoate | | | |
| tridecane-1,2-diol | 216.363 | 4.0271 | 40.46 |

In another aspect, the first penetration enhancer, the second penetration enhancer, or both can be any suitable ester as shown in Table D:

TABLE D
Ester Penetration Enhancers

| | | | Polar |
|---|---|---|---|
| | Molecular | | Surface |
| Name | Weight | cLogP | Area |
| octyl 2-hydroxybenzoate | 250.337 | 4.3596 | 46.53 |
| 2,5,7,8-tetramethyl-2-(4,8,12- | 549.233 | 9.9906 | 52.6 |
| trimethyltridecyl)-3,4-dihydro-2H-1- | | | |
| benzopyran-6-yl 4-chloro-4-oxobutanoate | | | |
| 4-{2-[2-(decanoyloxy)ethoxy]ethoxy}-4- | 360.445 | 3.2682 | 99.13 |
| oxobutanoic acid | | | |
| 4-[2,3-bis(decanoyloxy)propoxy]-4- | 500.67 | 1.06 | 116.2 |
| oxobutanoic acid | | | |
| 1,3-bis(acetyloxy)propan-2-yl acetate | 218.204 | 0.0102 | 78.9 |
| propan-2-yl tetradecanoate | 270.455 | 6.3472 | 26.3 |
| 1,10-diethyl decanedioate | 258.356 | 3.7282 | 52.6 |
| ethyl octadec-9-enoate | 310.52 | 7.5533 | 26.3 |
| methyl dodecanoate | 214.347 | 4.6728 | 26.3 |
| methyl 2-hydroxybenzoate | 152.149 | 1.2269 | 46.53 |
| octyl 4-hydroxybenzoate | 250.337 | 4.3596 | 46.53 |
| butyl acetate | 116.159 | 1.4439 | 26.3 |
| 2,3-dihydroxypropyl octanoate | 218.292 | 1.7674 | 66.76 |
| 2-(3,4-dihydroxyoxolan-2-yl)-2- | 346.462 | 2.701 | 96.22 |
| hydroxyethyl dodecanoate | | | |
| 2-(3,4-dihydroxyoxolan-2-yl)-2- | 430.623 | 5.4274 | 96.22 |
| hydroxyethyl octadecanoate | | | |
| 2-(3,4-dihydroxyoxolan-2-yl)-2- | 428.607 | 5.1752 | 96.22 |
| hydroxyethyl octadec-9-enoate | | | |
| 2,3-dihydroxypropyl dodecanoate | 274.399 | 3.585 | 66.76 |
| propan-2-yl hexadecanoate | 298.509 | 7.256 | 26.3 |
| 2-ethylhexyl 4-(dimethylamino)benzoate | 277.406 | 4.3656 | 29.54 |
| 1,2-dihydroxypropyl decanoate | 246.345 | 2.8952 | 66.76 |
| propan-2-yl decanoate | 214.347 | 4.5296 | 26.3 |
| 2-(3,4-dihydroxyoxolan-2-yl)-2- | 402.569 | 4.5186 | 96.22 |
| hydroxyethyl hexadecanoate | | | |
| 2,3-dihydroxypropyl icosanoate | 386.614 | 7.2202 | 66.76 |
| methyl propanoate | 88.1055 | 0.5832 | 26.3 |
| methyl 4-aminobenzoate | 151.164 | 0.8953 | 52.32 |
| methyl pentanoate | 116.159 | 1.492 | 26.3 |
| 2-(2-hydroxyethoxy)ethyl decanoate | 260.372 | 3.1511 | 55.76 |
| 4-[2-(2-hydroxyethoxy)ethoxy]-4- | 206.193 | −0.8516 | 93.06 |
| oxobutanoic acid | | | |
| propyl 4-hydroxybenzoate | 180.202 | 2.0876 | 46.53 |
| butyl 4-hydroxybenzoate | 194.229 | 2.542 | 46.53 |
| methyl 4-hydroxybenzoate | 152.149 | 1.2269 | 46.53 |

In another aspect, the first penetration enhancer, the second penetration enhancer, or both can be any suitable ether as shown in Table E:

TABLE E
Ether Penetration Enhancers

| | | | Polar |
|---|---|---|---|
| | Molecular | | Surface |
| Name | Weight | cLogP | Area |
| 1-methoxypropan-2-ol | 90.1214 | −0.089 | 29.46 |
| 2-(propan-2-yloxy)ethan-1-ol | 104.148 | 0.3173 | 29.46 |
| 2-methoxyethyl acetate | 118.131 | 0.0363 | 35.53 |
| 2-ethoxyethan-1-ol | 90.1214 | −0.042 | 29.46 |
| 2-butoxyethan-1-ol | 118.175 | 0.8668 | 29.46 |
| 2-(2-ethoxyethoxy)ethan-1-ol | 134.174 | −0.1345 | 38.69 |
| 2-phenoxyethan-1-ol | 138.165 | 1.0692 | 29.46 |
| 2-(2-butoxyethoxy)ethyl acetate | 204.265 | 1.2589 | 44.76 |
| 4-propoxyphenol | 152.192 | 2.1046 | 29.46 |
| 2-(octadecyloxy)ethan-1-ol | 314.551 | 7.2284 | 29.46 |
| 26-(4-nonylphenoxy)-3,6,9,12,15,18,21,24-octaoxahexacosan-1-ol | 616.829 | 4.2695 | 103.3 |
| 2-propoxyethan-1-ol | 104.148 | 0.4124 | 29.46 |
| 2-[2-(2-dodecoxyethoxy)ethoxy]ethanol | 318.496 | 4.317 | 47.92 |
| 3,6,9,12,15,18,21,24-octaoxahexatriacontan-1-ol | 538.76 | 3.8545 | 94.07 |
| 1-(3-methoxypropoxy)propan-1-ol | 148.201 | 0.587 | 38.69 |
| 2-octadec-9-enoxyethanol | 312.536 | 6.9762 | 29.46 |
| 1-(naphthalen-1-yloxy)-3-[(propan-2-yl)amino]propan-2-ol | 259.348 | 2.4155 | 41.49 |
| 2-[2-(hexadecyloxy)ethoxy]ethan-1-ol | 330.55 | 6.2271 | 38.69 |
| ethoxyethane | 74.1224 | 0.8847 | 9.23 |
| 3,6,9,12,15,18,21,24,27,30,33,36,39,42,45,48,51,54,57,60,63,66,69- | 1199.55 | 2.467 | 232.52 |
| tricosaoxahenoctacontan-1-ol | | | |
| 2-(dodecyloxy)ethan-1-ol | 230.39 | 4.502 | 29.46 |
| 2-(octadec-9-en-1-yloxy)ethan-1-ol | 312.536 | 6.9762 | 29.46 |

In another aspect, the first penetration enhancer, the second penetration enhancer, or both can be any suitable surfactant as shown in Table F:

TABLE F
Surfactant Penetration Enhancers

| | | | Polar |
|---|---|---|---|
| | Molecular | | Surface |
| Name | Weight | cLogP | Arca |
| 4-(2,4,4-trimethylpentan-2-yl)phenol; ethane-1,2-diol; formaldehyde | 4.5887 | −3.357 | 20.23 |
| 2-{2-[3,5-bis(2-hydroxyethoxy)oxolan-2-yl]-2-(2- | 5.3068 | −4.334 | 133.14 |
| hydroxyethoxy)ethoxy}ethyl octadec-9-enoate | | | |
| 4-hydroxy-2,5-bis(hydroxymethyl)-2-{[3,4,5-trihydroxy-6- | 3.1448 | −4.048 | 195.6 |
| (hydroxymethyl)oxan-2-yl]oxy}oxolan-3-yl octadecanoate | | | |
| 1,4-bis[(2-ethylhexyl)oxy]-1,4-dioxobutane-2-sulfonic acid | 3.6341 | −3.5 | 115.35 |
| dodecyltrimethylazanium | 1.8769 | −2.542 | 0 |
| trimethyl(tetradecy])azaninm | 2.7857 | −3.082 | 0 |
| tetrabutylazanium | 2.187 | −2.902 | 0 |
| sodium 2-(dodecyldimethylazaniumyl)acetate chloride | −1.0691 | −2.317 | 40.13 |
| N,N-dimethylheptanamide | 2.1931 | −1.439 | 20.31 |
| N,N-dimethyloctanamide | 2.6475 | −1.709 | 20.31 |
| sodium tetradecyl sulfate | 2.6409 | −2.571 | 74.81 |
| benzyldimethyl(2-{2-[4-(2,4,4-trimethylpentan-2- | 2.952 | −4.073 | 18.46 |
| yl)phenoxy]ethoxy}ethyl)azanium chloride | | | |
| benzyl(hexadecyl)dimethylazanium chloride | 5.1126 | −4.945 | 0 |
| dodecan-1-amine | 4.1967 | −3.253 | 26.02 |
| calcium bis(1,4-bis[(2-ethylhexyl)oxy]-1,4-dioxobutane-2-sulfonate) | 2.3865 | −3.5 | 118.18 |
| sodium 2-(N-methyldodecanamido)acetate | 1.5191 | −2.564 | 60.44 |
| dimethyldioctadecylazanium | 12.28 | −8.782 | 0 |
| 3-(dodecyldimethylazaniumyl)propane-1-sulfonate | −0.8103 | −2.336 | 65.58 |
| decyltrimethylazanium | 0.9681 | −2.002 | 0 |
| sodium dodecyl sulfate | 1.7321 | −2.031 | 74.81 |
| dodecylurea | 3.9824 | −3.714 | 55.12 |
| sodium 4-(dodecan-2-yl)benzene-1-sulfonate | 3.4593 | −3.523 | 65.58 |
| (2-{[3-(hexadecanoyloxy)-2-(octadec-9-enoyloxy)propyl | 6.6157 | −6.666 | 121 |
| phosphonato]oxy}ethyl)trimethylazanium | | | |
| 4-{[3-(butan-2-yl)-6,12-bis(hydroxy methyl)-22-methyl-9,15-bis(2- | 0.4778 | −6.108 | 386.19 |
| methylpropyl)-2,5,8,11,14,17,20-heptaoxo-18-(propan-2-y])-1-oxa- | | | |
| 4,7,10,13,16,19-hexaazacyclodocosan-21-yl]carbamoyl}-4-[2-(3- | | | |
| hydroxydecanamido)-4-methylpentanamido]butanoic acid | | | |
| 1,3-didodecylurea | 9.2842 | −6.63 | 41.13 |
| 2-[(3-dodecanamidopropyl)dimethylazaniumyl]acetate | −1.531 | −2.354 | 69.23 |
| 2-dodecylbenzene-1-sulfonic acid | 4.7338 | −3.425 | 62.75 |
| N,N-dimethylhexanamide | 1.7387 | −1.169 | 20.31 |
| N,N-dimethylnonanamide | 3.1019 | −1.979 | 20.31 |
| 3-dodecyl-1-phenylthiourea | 6.4393 | −5.221 | 56.15 |
| 1-dodecyl-3-methylthiourea | 4.7306 | −3.714 | 56.15 |
| hexadecyltrimethylazanium | 3.6945 | −3.622 | 0 |
| 2-{[6-(dodecyloxy)-4,5-dihydroxy-2-(hydroxymethyl)oxan-3-yl]oxy}- | 0.9203 | −2.577 | 178.53 |
| 6-(hydroxymethyl)oxane-3,4,5-triol | | | |
| (2-{[2,3-bis(icosanoyloxy)propyl | 9.5943 | −8.514 | 121 |
| phosphonato]oxy}ethyl)trimethylazanium | | | |
| 4-decyl-1,3-oxazolidin-2-one | 4.1983 | −3.72 | 38.33 |
| 1-hexadecylpyridin-1-ium | 3.3527 | −4.269 | 3.88 |
| 3,6,9,12,15,18,21,24,27,30,33,36,39,42,45,48,51,54,57,60- | 4.5621 | −2.677 | 204.83 |
| icosaoxahexaheptacontan-1-ol | | | |
| 2-{2-[3,4-bis(2-hydroxyethoxy)oxolan-2-yl]-2-(2- | 2.331 | −2.729 | 133.14 |
| hydroxyethoxy)ethoxy}ethyl dodecanoate | | | |
| benzyldimethyloctylazanium chloride | 1.4774 | −2.785 | 0 |

In another aspect, the first penetration enhancer, the second penetration enhancer, or both can be selected from the group consisting of: dimethyl isosorbide, diethylene glycol monoethyl ether, ethylene glycol monododecyl ether, adipic acid diisopropyl ester, diethyl sebacate, and combinations thereof. In one example, the first penetration enhancer or the second penetration enhancer can be dimethyl isosorbide.

In one aspect, the at least one cannabinoid receptor modulator and the dimethyl isosorbide can be present in a ratio of from about 20:1 to about 1:20. In another aspect, the first penetration enhancer and the dimethyl isosorbide can be present in a ratio of from about 1:10 to about 10:1.

In some embodiments, the first penetration enhancer, the second penetration enhancer, or both can comprise a 16-carbon saturated fatty acid. In some embodiments, the first penetration enhancer, the second penetration enhancer, or both can comprise, but are not limited to, one or more of: a hexadecanoic acid, a salt of hexadecenoic acid, an ester of hexadecenoic acid, the like, or a combination thereof. In one example, the first penetration enhancer, the second penetration enhancer, or both can comprise, but is not limited to, one or more of: propan-2-yl hexadecanoate, 2-(3,4-dihydroxyoxolan-2-yl)-2-hydroxyethyl hexadecanoate, (6-{[3,4-dihydroxy-2,5-bis(hydroxymethyl)oxolan-2-yl]oxy}-3,4,5-trihydroxyoxan-2-yl)methyl hexadecanoate, hexadecanoic acid, 2-(2-oxoazepan-1-yl)hexadecanoic acid, 1-(2,6-dimethylmorpholin-4-yl)hexadecan-1-one, 1-(4-hexadecanoylpiperazin-1-yl)hexadecan-1-one, 1-(4-methylpiperazin-1-yl)hexadecan-1-one, 1-(azepan-1-yl)hexadecan-1-one, 1-(morpholin-4-yl)hexadecan-1-one, 1-(piperazin-1-yl)hexadecan-1-one, hexadecan-1-ol, the like, or combinations thereof. In another example, the first penetration enhancer, the second penetration enhancer, or both can be cetyl palmitate, sorbitan palmitate, the like, or a combination thereof. In one aspect, the first penetration enhancer and second penetration enhancers can be present in a ratio of from about 10:1 to about 1:50.

In another aspect, the combination of the first and second penetration enhancers can be present in the topical composition at a concentration of from about 0.0001 wt % to about 80 wt % of the composition.

In one aspect, the first penetration enhancer (e.g., cetyl palmitate, sorbitan palmitate) can be present in the topical composition at a concentration of from about 0.0001 wt % to about 50 wt %. In another aspect, the first penetration enhancer (e.g., cetyl palmitate, sorbitan palmitate) can be present in the topical composition at a concentration of from about 1 wt % to about 10 wt %. In another aspect, the first penetration enhancer (e.g., cetyl palmitate, sorbitan palmitate) can be present in the topical composition at a concentration of from about 1 wt % to about 6 wt %.

In another aspect, the second penetration enhancer (e.g., a terpene or dimethyl isosorbide) can be present in the topical composition at a concentration of from about 2 wt % to about 20 wt %. In another aspect, the second penetration enhancer (e.g., a terpene or dimethyl isosorbide) can be present in the topical composition at a concentration of from about 4 wt % to about 12 wt %.

In some embodiments, the first penetration enhancer can comprise one or more of: a hexadecanoic acid, a salt of hexadecenoic acid, an ester of hexadecenoic acid, the like, or a combination thereof (e.g., cetyl palmitate, sorbitan palmitate), and a second penetration enhancer can be amphiphilic (e.g., dimethyl isosorbide). In one example, the topical composition can comprise an additional penetration enhancer that can comprise a terpene (e.g., biasbolol, R-caryophyllene, D-limonene, linalool, nerolidol, myrcene, terpinolene).

In some embodiments, the first penetration enhancer can comprise one or more of: a hexadecanoic acid, a salt of hexadecenoic acid, an ester of hexadecenoic acid, the like, or a combination thereof (e.g., cetyl palmitate, sorbitan palmitate), and a second penetration enhancer can be a terpene (e.g., biasbolol, D-caryophyllene, D-limonene, linalool, nerolidol, myrcene, terpinolene).

In some embodiments, the first penetration enhancer can comprise one or more of: a hexadecanoic acid, a salt of hexadecenoic acid, an ester of hexadecenoic acid, the like, or a combination thereof (e.g., cetyl palmitate, sorbitan palmitate), and a second penetration enhancer can be amphiphilic (e.g., dimethyl isosorbide).

In some embodiments, the topical composition can comprise an additional penetration enhancer. In some aspects, the additional penetration enhancer can be one or more of: (a) aliphatic, (b) aromatic, (c) an azepane, (d) an azone, (e) a caprolactam, (f) a fatty acid, (g) a morpholine, (h) a piperazine, (i) a piperidine, (j) a pryyolidine, (k) a steroid, (l) a cuccinimide, (m) penetration enhancers disclosed herein, the like, or a combination thereof, as shown in Tables G to R.

In another aspect, the first penetration enhancer, the second penetration enhancer, or both can be any suitable aliphatic as shown in Table G:

TABLE G
Aliphatic Penetration Enhancers

| | | | Polar |
|---|---|---|---|
| | Molecular | | Surface |
| Name | Weight | cLogP | Area |
| 2-heptyl-2-methyl-1,3-dioxolane | 186.294 | 3.199 | 18.46 |
| 2,4-dimethyl-2-nonyl-1,3-dioxolane | 228.374 | 4.4325 | 18.46 |
| 2-methyl-2-undecyl-1,3-dioxolane | 242.401 | 5.0166 | 18.46 |
| alpha-cyclodextrin | 972.842 | −11.023 | 474.9 |
| 2-chloroacetonitrile | 75.4979 | 0.4521 | 23.79 |
| 2-[(phosphonomethyl)amino]acetic acid | 169.073 | −6.2784 | 116.67 |
| 8-methyl-6,9-diazaspiro[4.5]decane- | 182.222 | −0.3572 | 58.2 |
| 7,10-dione | | | |
| 2-[(2- | 134.134 | −1.1784 | 73.13 |
| hydroxyethyl)(nitroso)amino]ethan-1-ol | | | |
| ethyl acetate | 88.1055 | 0.5351 | 26.3 |
| 2-butyl-2-methyl-1,3-dioxolane | 144.213 | 1.8358 | 18.46 |
| 2-heptyl-2,4-dimethyl-1,3-dioxolane | 200.321 | 3.5237 | 18.46 |
| 7-oxabicyclo[4.1.0]heptane | 98.1444 | 0.9022 | 12.53 |
| 2,2-dichloroacetonitrile | 109.943 | 0.8543 | 23.79 |
| 2,2-dibromoacetonitrile | 198.845 | 1.1163 | 23.79 |
| 2-methyl-2-nonyl-1,3-dioxolane | 214.347 | 4.1078 | 18.46 |
| 2-nonyl-1,3-dioxolane | 200.321 | 3.8435 | 18.46 |
| 2-heptyl-1,3-dioxolane | 172.267 | 2.9347 | 18.46 |
| 1,1-dichloropropan-2-one | 126.97 | 1.0101 | 17.07 |
| 4-methyl-2-nonyl-1,3-dioxolane | 214.347 | 4.1682 | 18.46 |
| 1,3-bis(nitrooxy)propan-2-yl nitrate | 227.085 | −2.3418 | 165.15 |
| 2-bromo-2-chloroacetic acid | 173.393 | 0.4634 | 37.3 |
| Urea | 60.0558 | −1.3194 | 69.11 |
| 2-undecyl-1,3-dioxolane | 228.374 | 4.7523 | 18.46 |
| ethyldimethylamine | 73.1383 | 0.2707 | 3.24 |
| 2,2-dibromoacetic acid | 217.844 | 0.5944 | 37.3 |
| acetic acid | 60.0518 | −0.2991 | 37.3 |
| methanesulfinylmethane | 78.1346 | −1.2837 | 36.28 |
| 1,1,1-trichloroethane | 133.405 | 2.4217 | 0 |
| 2-heptyl-4-methyl-1,3-dioxolane | 186.294 | 3.2594 | 18.46 |
| bromodichloromethane | 163.829 | 1.7658 | 0 |
| trimethylamine | 59.1115 | −0.1356 | 3.24 |
| trichloroacetic acid | 163.387 | 1.1454 | 37.3 |
| butan-2-one | 72.1065 | 0.833 | 17.07 |
| 2-bromoacetic acid | 138.948 | 0.0612 | 37.3 |
| 2-chloroacetic acid | 94.4968 | −0.0698 | 37.3 |
| methyl acetate | 74.0786 | 0.1288 | 26.3 |
| 2,2-dichloroacetic acid | 128.942 | 0.3324 | 37.3 |
| 4-methyl-2-undecyl-1,3-dioxolane | 242.401 | 5.077 | 18.46 |
| 2-bromo-2-chloroacetonitrile | 154.394 | 0.9853 | 23.79 |
| 1,1,1-trichloropropan-2-one | 161.415 | 1.8231 | 17.07 |
| 2-hexyl-2-methyl-1,3-dioxolane | 172.267 | 2.7446 | 18.46 |

In another aspect, the first penetration enhancer, the second penetration enhancer, or both can be any suitable aromatic as shown in Table H:

TABLE H
Aromatic Penetration Enhancers

| | | | Polar |
|---|---|---|---|
| | Molecular | | Surface |
| Name | Weight | cLogP | Area |
| ethylbenzene | 139.11 | 0.3923 | 66.05 |
| ethenylbenzene | 106.167 | 2.4191 | 0 |
| phenylmethanol | 104.152 | 2.3625 | 0 |
| benzaldehyde | 108.14 | 1.0623 | 20.23 |
| anisole | 106.124 | 1.5929 | 17.07 |
| 1,3-diphenylurea | 108.14 | 1.5896 | 9.23 |
| 1,3-diphenylthiourea | 212.251 | 2.801 | 41.13 |
| 4-bromophenol | 228.318 | 3.1977 | 56.15 |
| 4-methylphenol | 173.009 | 2.0391 | 20.23 |
| 4-chlorophenol | 108.14 | 1.6578 | 20.23 |
| benzene-1,4-diamine | 128.558 | 1.9199 | 20.23 |
| 3-methylphenol | 108.144 | 0.305 | 52.04 |
| benzene-1,3-diol | 108.14 | 1.6578 | 20.23 |
| benzene-1,3,5-triol | 110.112 | 0.9682 | 40.46 |
| toluene | 126.111 | 0.6225 | 60.69 |
| phenol | 92.1405 | 2.1935 | 0 |
| 2-phenylpropan-1-ol | 94.1126 | 1.3139 | 20.23 |
| 4-amino-2-nitrophenol | 136.193 | 1.9199 | 20.23 |
| isoquinoline | 154.125 | −0.285 | 92.07 |
| benzene-1,2-diol | 129.162 | 1.8531 | 12.89 |
| 2,4-dichlorophenol | 110.112 | 0.9682 | 40.46 |
| 2-tert-butyl-4-methoxyphenol | 163.003 | 2.5259 | 20.23 |
| 4-ethylphenol | 180.246 | 2.8258 | 29.46 |
| benzene-1,4-diol | 122.166 | 2.0734 | 20.23 |
| methyl 2-(4-hydroxyphenyl)acetate | 110.112 | 0.9682 | 40.46 |
| 2-(4-hydroxyphenyl)acetic acid | 166.175 | 1.225 | 46.53 |
| 4-butylphenol | 152.149 | 0.7971 | 57.53 |
| 2-(4-hydroxyphenyl)acetamide | 150.22 | 2.9822 | 20.23 |
| 2-phenylbutan-1-ol | 151.164 | 0.3993 | 63.32 |
| 2-[4-(2,4,4-trimethylpentan-2- | 150.22 | 2.3743 | 20.23 |
| yl)phenoxyl]ethan-1-ol | | | |
| 4-methyl-2-(propan-2-yl)phenol | 250.38 | 4.344 | 29.46 |
| 2-nitrobenzene-1,4-diamine | 150.22 | 2.8448 | 20.23 |
| benzene-1,2,4-triol | 153.141 | −0.6166 | 97.86 |
| 3-nitrophenol | 126.111 | 0.6225 | 60.69 |
| 4-chloro-3-methylphenol | 139.11 | 0.3923 | 66.05 |
| 2-phenylethan-1-ol | 142.585 | 2.2638 | 20.23 |
| aniline | 122.166 | 1.4924 | 20.23 |
| 4-(hydroxymethyl)phenol | 93.1286 | 0.9823 | 26.02 |
| 2-phenylethan-1-amine | 124.139 | 0.7166 | 40.46 |
| benzoic acid | 121.182 | 1.0946 | 26.02 |
| benzene | 122.123 | 1.1447 | 37.3 |
| 4-hydroxybenzonitrile | 78.1136 | 1.6596 | 0 |
| benzene-1,2,3-triol | 119.123 | 1.1495 | 44.02 |
| 4-chloro-3,5-dimethylphenol | 126.111 | 0.6225 | 60.69 |
| 2,4,6-trichlorophenol | 156.611 | 2.6077 | 20.23 |
| 2-tert-butylphenol | 197.448 | 3.1319 | 20.23 |
| 2-hydroxybenzaldehyde | 150.22 | 2.8958 | 20.23 |
| 2-methylphenol | 122.123 | 1.2472 | 37.3 |
| benzene-1,2-diamine | 108.14 | 1.6578 | 20.23 |
| 2-chlorophenol | 108.144 | 0.305 | 52.04 |
| 3,4-dimethylphenol | 128.558 | 1.9199 | 20.23 |
| 4-chlorobenzene-1,2-diamine | 122.166 | 2.2017 | 20.23 |
| 2-amino-4-nitrophenol | 142.589 | 0.911 | 52.04 |

In another aspect, the first penetration enhancer, the second penetration enhancer, or both can be any suitable azepane as shown in Table I:

TABLE I
Azepane Penetration Enhancers

| | | | |
|---|---|---|---|
| 1-(azepan-1-yl)non-8-en-1-one | 237.385 | 4.3914 | 20.31 |
| 1-(azepan-1-yl)dec-9-en-1-one | 251.412 | 4.8458 | 20.31 |
| 1-(azepan-1-yl)undec-10-en-1-one | 265.439 | 5.3002 | 20.31 |
| 1-(2,6-dimethylazepan-1-yl)dodec-10-en-1-one | 307.52 | 6.2819 | 20.31 |
| 1-(azepan-1-yl)tetradec-13-en-1-one | 307.52 | 6.6634 | 20.31 |
| 1-(2,6-dimethylazepan-1-yl)tridec-11-en-1-one | 321.547 | 6.7363 | 20.31 |
| 1-(azepan-1-yl)pentadec-14-en-1-one | 321.547 | 7.1178 | 20.31 |
| 1-(2,6-dimethylazepan-1-yl)tetradec-12-en- | 335.574 | 7.1907 | 20.31 |
| 1-one | | | |
| 1-(azepan-1-yl)hexadec-15-en-1-one | 335.574 | 7.5722 | 20.31 |
| 1-(2,6-dimethylazepan-1-yl)hexadec-10-en- | 363.627 | 8.0995 | 20.31 |
| 1-one | | | |
| 1-(azepan-1-yl)octadec-17-en-1-one | 363.627 | 8.481 | 20.31 |
| 1-(2,6-dimethylazepan-1-yl)octadec-10-en- | 391.681 | 9.1983 | 20.31 |
| 1-one | | | |
| 1-decylazepane | 239.445 | 5.3809 | 3.24 |
| 1-octadecylazepane | 351.66 | 9.0161 | 3.24 |
| 1-hexadecylazepane | 323.606 | 8.1073 | 3.24 |
| 1-(azepan-1-yl)octan-1-one | 225.374 | 4.1225 | 20.31 |
| 1-(azepan-1-yl)dodecan-1-one | 281.482 | 5.9401 | 20.31 |
| 1-dodecylazepane | 267.499 | 6.2897 | 3.24 |
| 1-(azepan-1-yl)hexadecan-1-one | 337.589 | 7.7577 | 20.31 |
| 1-undecylazepane | 253.472 | 5.8353 | 3.24 |
| 1-tetradecylazepane | 295.553 | 7.1985 | 3.24 |

In another aspect, the first penetration enhancer, the second penetration enhancer, or both can be any suitable azone as shown in Table J:

TABLE J
Azone Penetration Enhancers

| | | | |
|---|---|---|---|
| 1-dodecylazepane-2-thione | 297.549 | 5.9429 | 35.33 |
| 1-butylpyrrolidine-2-thione | 157.28 | 1.6237 | 35.33 |
| 1-octylpyrrolidine-2-thione | 213.388 | 3.4413 | 35.33 |
| 1-dodecylpyrrolidine-2-thione | 269.495 | 5.2589 | 35.33 |
| 1-dodecylpiperidine-2-thione | 283.522 | 5.6009 | 35.33 |
| 1-[7-(2-oxopyrrolidin-1-yl)heptyl]pyrrolidin-2-one | 266.383 | 2.2858 | 40.62 |
| 1-dodecylpyrrolidine-2,4-dione | 267.411 | 4.3311 | 37.38 |
| 4-dodecylmorpholine-3,5-dione | 283.41 | 3.6513 | 46.61 |
| 4-tetradecylmorpholine-3,5-dione | 311.464 | 4.5601 | 46.61 |
| 4-methoxy-1-methyl-2,3-dihydro-1H-azepin-2-one | 153.18 | 0.1584 | 29.54 |
| 4-acetyl-1-methyl-2,3-dihydro-1H-azepin-2-one | 165.191 | 0.0719 | 37.38 |
| N,N-diethyl-1-methyl-2-oxo-2,3-dihydro-1H-azepine-4-carboxamide | 222.287 | 0.427 | 40.62 |
| 1-methyl-4-phenyl-2,3-dihydro-1H-azepin-2-one | 199.252 | 1.4855 | 20.31 |
| 4-decyl-1,4-oxazepane-5,7-dione | 269.383 | 3.0845 | 46.61 |
| 1-dodecyl-2,7-dihydro-1H-azepin-2-one | 277.45 | 5.1727 | 20.31 |
| 1-dodecyl-2,5,6,7-tetrahydro-1H-azepin-2-one | 279.466 | 5.4481 | 20.31 |
| 1-dodecyl-2,3,6,7-tetrahydro-1H-azepin-2-one | 279.466 | 5.4481 | 20.31 |
| 1-[3-(2-oxoazepan-1-yl)butyl]azepan-2-one | 280.41 | 2.1955 | 40.62 |
| 1-[8-(2-oxopyrrolidin-1-yl)octyl]pyrrolidin-2-one | 280.41 | 2.7402 | 40.62 |
| 1-(thiomorpholin-4-yl)dodecan-1-one | 285.494 | 4.841 | 45.61 |
| 1-dodecanoyl-2,5,6,7-tetrahydro-1H-azepin-2-one | 293.449 | 5.0985 | 37.38 |
| 1-dodecanoyl-2,3,6,7-tetrahydro-1H-azepin-2-one | 293.449 | 5.0985 | 37.38 |
| 4-dodecyl-1,4-oxazepane-5,7-dione | 297.437 | 3.9933 | 46.61 |
| 1-dodecylazepane-2,7-dione | 295.465 | 5.1573 | 37.38 |
| 1-[6-(2-oxopyrrolidin-1-yl)hexyl]pyrrolidin-2-one | 252.357 | 1.8314 | 40.62 |
| 1-phenylpyrrolidin-2-one | 161.203 | 1.5844 | 20.31 |
| 1-benzylpyrrolidin-2-one | 175.23 | 1.5073 | 20.31 |
| 2-decylcyclopentan-1-one | 224.386 | 5.3605 | 17.07 |
| 1,6-bis(morpholin-4-yl)hexane-1,6-dione | 284.355 | 0.6206 | 59.08 |
| 2-decylcyclohexan-1-one | 238.413 | 5.7025 | 17.07 |
| 1-butylazepane-2-thione | 185.334 | 2.3077 | 35.33 |
| 1-octylazepane-2-thione | 241.441 | 4.1253 | 35.33 |
| 1-cyclohexylpyrrolidin-2-one | 167.251 | 1.6449 | 20.31 |
| 1,6-bis(thiomorpholin-4-yl)hexane-1,6-dione | 316.489 | 1.4344 | 91.22 |

In another aspect, the first penetration enhancer, the second penetration enhancer, or both can be any suitable caprolactam as shown in Table K:

TABLE K
Caprolactam Penetration Enhancers

| | | | |
|---|---|---|---|
| 1-methyl-3-(2-oxopyrrolidin-1-yl)azepan-2-one | 210.276 | 0.3661 | 40.62 |
| methyl 2-[2-oxo-3-(2-oxopyrrolidin-1-yl)azepan-1-yl]acetate | 268.312 | −0.076 | 66.92 |
| 1-hexyl-3-(2-oxopyrrolidin-1-yl)azepan-2-one | 280.41 | 2.59 | 40.62 |
| 1-decyl-3-(2-oxopyrrolidin-1-yl)azepan-2-one | 336.518 | 4.4076 | 40.62 |
| hexyl 2-[2-oxo-3-(2-oxopyrrolidin-1-yl)azepan-1-yl]acetate | 338.446 | 2.1479 | 66.92 |
| 1-dodecyl-3-(2-oxopyrrolidin-1-y])azepan-2-one | 364.572 | 5.3164 | 40.62 |
| 3-(2-oxopyrrolidin-1-yl)-1-tetradecylazepan-2-one | 392.625 | 6.2252 | 40.62 |
| decyl 2-12-oxo-3-(2-oxopyrrolidin-1-yl)azepan-1-yl]acetate | 394.554 | 3.9655 | 66.92 |
| 1-hexadecyl-3-(2-oxopyrrolidin-1-y])azepan-2-one | 420.679 | 7.134 | 40.62 |
| dodecyl 2-12-oxo-3-(2-oxopyrrolidin-1-yl)azepan-1-ylacetate | 422.607 | 4.8743 | 66.92 |
| tetradecyl 2-[2-oxo-3-(2-oxopyrrolidin-1-yl)azepan-1-yl]acetate | 450.661 | 5.7831 | 66.92 |
| 1-(3-hydroxypropyl)azepan-2-one | 171.239 | 0.7072 | 40.54 |
| 1-(2,3-dihydroxypropyl)azepan-2-one | 187.238 | −0.3146 | 60.77 |
| 1-(pent-4-enoyl)azepan-2-one | 195.261 | 2.1976 | 37.38 |
| 1-(hex-5-enoyl)azepan-2-one | 209.288 | 2.462 | 37.38 |
| 1-heptylazepan-2-one | 211.348 | 3.4515 | 20.31 |
| 1-(hept-6-enoy])azepan-2-one | 223.315 | 2.9164 | 37.38 |
| 2-(2-oxoazepan-1-yl)hexanoic acid | 227.303 | 1.6257 | 57.61 |
| 1-(oct-7-enoyl)azepan-2-one | 237.342 | 3.3708 | 37.38 |
| 2-(2-oxoazepan-1-yl)heptanoic acid | 241.33 | 2.0801 | 57.61 |
| 1-(non-8-enoyl)azepan-2-one | 251.369 | 3.8252 | 37.38 |
| 2-(2-oxoazepan-1-yl)octanoic acid | 255.356 | 2.5345 | 57.61 |
| 1-(dec-9-enoyl)azepan-2-one | 265.395 | 4.2796 | 37.38 |
| 1-decanoylazepan-2-one | 267.411 | 4.4651 | 37.38 |
| 2-(2-oxoazepan-1-yl)nonanoic acid | 269.383 | 2.9889 | 57.61 |
| 2-(2-oxoazepan-1-yl)decanoic acid | 283.41 | 3.4433 | 57.61 |
| 2-(2-oxoazepan-1-yl)undecanoic acid | 297.437 | 3.8977 | 57.61 |
| 2-(2-oxoazepan-1-yl)dodecanoic acid | 311.464 | 4.3521 | 57.61 |
| 1-(tetradec-13-enoyl)azepan-2-one | 321.503 | 6.0972 | 37.38 |
| 1-tetradecanoylazepan-2-one | 323.519 | 6.2827 | 37.38 |
| 1-(3,7,11-trimethyldodecyl)azepan-2-one | 323.563 | 6.3784 | 20.31 |
| 2-(2-oxoazepan-1-yl)tetradecanoic acid | 339.518 | 5.2609 | 57.61 |
| dodecyl 2-(2-oxoazepan-1-yl)acetate | 339.518 | 5.2814 | 46.61 |
| 1-(hexadec-15-enoyl)azepan-2-one | 349.557 | 7.006 | 37.38 |
| 2-(2-oxoazepan-1-yl)hexadecanoic acid | 367.572 | 6.1697 | 57.61 |
| tetradecyl 2-(2-oxoazepan-1-yl)acetate | 367.572 | 6.1902 | 46.61 |
| 1-(octadec-17-enoy])azepan-2-one | 377.61 | 7.9148 | 37.38 |
| 2-(2-oxoazepan-1-yl)octadecanoic acid | 395.625 | 7.0785 | 57.61 |
| 1-(3,7-dimethyloctyl)azepan-2-one | 253.428 | 4.3425 | 20.31 |
| 1-tetradecylazepan-2-one | 309.536 | 6.6323 | 20.31 |
| 1-hexadecylazepan-2-one | 337.589 | 7.5411 | 20.31 |
| 1-(3,7-dimethylocta-2,6-dien-1-yl)azepan-2-one | 249.396 | 4.6143 | 20.31 |
| 1-(3,7,11-trimethyldodeca-2,6,10-trien-1-yl)azepan-2-one | 317.515 | 6.7861 | 20.31 |
| 1-(3,7,11,15-tetramethylhexadeca-2,6,10,14-tetraen-1-yl)azepan-2-one | 385.633 | 8.9579 | 20.31 |
| 1-acetylazepan-2-one | 155.196 | 0.8299 | 37.38 |
| 1-butylazepan-2-one | 169.267 | 2.0883 | 20.31 |
| 1-methylazepan-2-one | 127.186 | 0.7732 | 20.31 |
| 1-(undec-10-enoyl)azepan-2-one | 279.422 | 4.734 | 37.38 |
| 1-hexylazepan-2-one | 197.321 | 2.9971 | 20.31 |
| 1-(2-hydroxyethyl)azepan-2-one | 157.212 | 0.2528 | 40.54 |
| 1-propylazepan-2-one | 155.24 | 1.6339 | 20.31 |
| 1-dodecanoylazepan-2-one | 295.465 | 5.3739 | 37.38 |
| 1-octylazepan-2-one | 225.374 | 3.9059 | 20.31 |
| 1-dodecylazepan-2-one | 281.482 | 5.7235 | 20.31 |
| 1-pentylazepan-2-one | 183.294 | 2.5427 | 20.31 |
| 1-nonylazepan-2-one | 239.401 | 4.3603 | 20.31 |
| 1-decylazepan-2-one | 253.428 | 4.8147 | 20.31 |

In another aspect, the first penetration enhancer, the second penetration enhancer, or both can be any suitable fatty acid as shown in Table L:

TABLE L
Fatty Acid Penetration Enhancers

| | | | |
|---|---|---|---|
| pentadecanoic acid | 242.401 | 5.6081 | 37.3 |
| butanoic acid | 88.1055 | 0.6097 | 37.3 |
| pentanoic acid | 102.132 | 1.0641 | 37.3 |
| heptanoic acid | 130.186 | 1.9729 | 37.3 |
| nonanoic acid | 158.24 | 2.8817 | 37.3 |
| undecanoic acid | 186.294 | 3.7905 | 37.3 |
| octadec-9-enoic acid | 282.466 | 6.7191 | 37.3 |
| docosanoic acid | 340.589 | 8.7889 | 37.3 |
| docos-13-enoic acid | 338.573 | 8.5367 | 37.3 |
| 2,2-dimethylpentanoic acid | 130.186 | 1.8482 | 37.3 |
| octanoic acid | 144.213 | 2.4273 | 37.3 |
| 12-hydroxyoctadec-9-enoic acid | 298.465 | 5.6973 | 57.53 |
| hexanoic acid | 116.159 | 1.5185 | 37.3 |
| 2,2-dimethylheptanoic acid | 158.24 | 2.757 | 37.3 |
| dodecanoic acid | 200.321 | 4.2449 | 37.3 |
| 2-heptylundecanoic acid | 284.482 | 6.7352 | 37.3 |
| 2,2-dimethyloctanoic acid | 172.267 | 3.2114 | 37.3 |
| decanoic acid | 172.267 | 3.3361 | 37.3 |
| hexadec-9-enoic acid | 254.412 | 5.8103 | 37.3 |
| octadeca-9,12,15-trienoic acid | 278.434 | 6.2147 | 37.3 |
| octadec-11-enoic acid | 282.466 | 6.7191 | 37.3 |
| icosanoic acid | 312.536 | 7.8801 | 37.3 |
| tetradecanoic acid | 228.374 | 5.1537 | 37.3 |
| tetradec-9-enoic acid | 226.358 | 4.9015 | 37.3 |
| icos-11-enoic acid | 310.52 | 7.6279 | 37.3 |
| tetracosanoic acid | 368.643 | 9.6977 | 37.3 |
| hexadecanoic acid | 256.428 | 6.0625 | 37.3 |
| octadecanoic acid | 284.482 | 6.9713 | 37.3 |
| octadec-6-enoic acid | 282.466 | 6.7191 | 37.3 |
| octadeca-9,12-dienoic acid | 280.45 | 6.4669 | 37.3 |
| tridecanoic acid | 214.347 | 4.6993 | 37.3 |

In another aspect, the first penetration enhancer, the second penetration enhancer, or both can be any suitable morpholine as shown in Table M:

TABLE M
Morpholine Penetration Enhancers

| | | | |
|---|---|---|---|
| 1-(morpholin-4-yl)hept-6-en-1-one | 197.277 | 1.9766 | 29.54 |
| 1-(morpholin-4-yl)oct-7-en-1-one | 211.304 | 2.431 | 29.54 |
| 1-(morpholin-4-yl)non-8-en-1-one | 225.331 | 2.8854 | 29.54 |
| 1-(morpholin-4-yl)deca-2,3-dien-1-one | 237.342 | 2.8187 | 29.54 |
| 1-(morpholin-4-yl)dec-9-en-1-one | 239.358 | 3.3398 | 29.54 |
| 1-(morpholin-4-yl)undec-2-en-1-one | 253.384 | 3.7275 | 29.54 |
| 1-(2,6-dimethylmorpholin-4-yl)decan-1-one | 269.427 | 4.1747 | 29.54 |
| 1-(morpholin-4-yl)tridec-12-en-1-one | 281.438 | 4.703 | 29.54 |
| 1-(2,6-dimethylmorpholin-4-yl)undecan-1-one | 283.454 | 4.6291 | 29.54 |
| 1-(2,6-dimethylmorpholin-4-yl)dodec-10-en-1- | 295.465 | 4.8313 | 29.54 |
| one | | | |
| 1-(morpholin-4-yl)tetradec-13-en-1-one | 295.465 | 5.1574 | 29.54 |
| 1-(2,6-dimethylmorpholin-4-yl)dodecan-1-one | 297.481 | 5.0835 | 29.54 |
| 1-(morpholin-4-yl)pentadec-14-en-1-one | 309.492 | 5.6118 | 29.54 |
| 1-(2,6-dimethylmorpholin-4-yl)tridecan-1-one | 311.508 | 5.5379 | 29.54 |
| 1-(morpholin-4-yl)pentadecan-1-one | 311.508 | 5.7973 | 29.54 |
| 1-(2,6-dimethylmorpholin-4-yl)tetradec-12-en- | 323.519 | 5.7401 | 29.54 |
| 1-one | | | |
| 1-(morpholin-4-yl)hexadec-15-en-1-one | 323.519 | 6.0662 | 29.54 |
| 1-(2,6-dimethylmorpholin-4-yl)tetradecan-1- | 325.535 | 5.9923 | 29.54 |
| one | | | |
| 1-(morpholin-4-yl)heptadecan-1-one | 339.562 | 6.7061 | 29.54 |
| 1-(2,6-dimethylmorpholin-4-yl)hexadec-10-en- | 351.573 | 6.6489 | 29.54 |
| 1-one | | | |
| 1-(morpholin-4-yl)octadec-17-en-1-one | 351.573 | 6.975 | 29.54 |
| 1-(2,6-dimethylmorpholin-4-yl)hexadecan-1- | 353.588 | 6.9011 | 29.54 |
| one | | | |
| 1-(2,6-dimethylmorpholin-4-yl)octadec-10-en- | 379.626 | 7.5577 | 29.54 |
| 1-one | | | |
| 1-(2,6-dimethylmorpholin-4-yl)octadecan-1- | 381.642 | 7.8099 | 29.54 |
| one | | | |
| 2-(morpholin-4-yl)hexanoic acid | 201.265 | −0.2406 | 49.77 |
| 2-(morpholin-4-yl)octanoic acid | 229.319 | 0.6682 | 49.77 |
| 2-(morpholin-4-yl)decanoic acid | 257.372 | 1.577 | 49.77 |
| 2-[2-(4-propylheptyl)morpholin-4-yl]ethan- | 271.443 | 3.4912 | 32.7 |
| 1-ol | | | |
| 2-(morpholin-4-yl)dodecanoic acid | 285.426 | 2.4858 | 49.77 |
| 2-(morpholin-4-yl)tetradecanoic acid | 313.48 | 3.3946 | 49.77 |
| 1-(morpholin-4-yl)dodecane-1-thione | 285.494 | 4.9071 | 44.56 |
| 4-dodecylmorpholine-2,3-dione | 283.41 | 3.6513 | 46.61 |
| 4-tetradecylmorpholine-2,3-dione | 311.464 | 4.5601 | 46.61 |
| 1-(morpholin-4-yl)tridecan-1-one | 283.454 | 4.8885 | 29.54 |
| 4-methylmorpholine | 101.148 | −0.1666 | 12.47 |
| 4-dodecylmorpholine | 255.444 | 4.7837 | 12.47 |
| 1-(morpholin-4-yl)undecan-1-one | 255.4 | 3.9797 | 29.54 |
| 1-(morpholin-4-yl)undec-10-en-1-one | 253.384 | 3.7942 | 29.54 |
| 1-(morpholin-4-yl)octadecan-1-one | 353.588 | 7.1605 | 29.54 |
| 1-(morpholin-4-yl)decan-1-one | 241.373 | 3.5253 | 29.54 |
| 1-(morpholin-4-yl)dodecan-1-one | 269.427 | 4.4341 | 29.54 |
| 1-(morpholin-4-yl)hexadecan-1-one | 325.535 | 6.2517 | 29.54 |
| 1-(morpholin-4-yl)tetradecan-1-one | 297.481 | 5.3429 | 29.54 |
| 2-[3-(4-propylheptyl)morpholin-4-yl]ethan- | 271.443 | 3.4912 | 32.7 |
| 1-ol | | | |

In another aspect, the first penetration enhancer, the second penetration enhancer, or both can be any suitable piperazine as shown in Table N:

TABLE N
Piperazine Penetration Enhancers

| | | | |
|---|---|---|---|
| 1-(4-methylpiperazin-1-yl)decan-1-one | 254.416 | 3.6347 | 23.55 |
| 1-(piperazin-1-yl)dodecan-1-one | 268.443 | 4.2906 | 32.34 |
| 1-(4-methylpiperazin-1-yl)dodecan-1-one | 282.47 | 4.5435 | 23.55 |
| 1-(4-methylpiperazin-1-yl)tetradecan-1-one | 310.524 | 5.4523 | 23.55 |
| 1-(piperazin-1-yl)hexadecan-1-one | 324.551 | 6.1082 | 32.34 |
| 1-(4-methylpiperazin-1-yl)hexadecan-1-one | 338.577 | 6.3611 | 23.55 |
| 1-(piperazin-1-yl)heptadecan-1-one | 338.577 | 6.5626 | 32.34 |
| 1-(4-octanoylpiperazin-1-yl)octan-1-one | 338.534 | 5.509 | 40.62 |
| 1-(4-decanoylpiperazin-1-yl)decan-1-one | 394.641 | 7.3266 | 40.62 |
| 1-(4-hexadecanoylpiperazin-1-yl)hexadecan-1- | 562.964 | 12.779 | 40.62 |
| one | | | |
| 1,6-bis(4-methylpiperazin-1-yl)hexane-1,6- | 310.44 | 0.8394 | 47.1 |
| dione | | | |
| 1,7-bis(4-methylpiperazin-1-yl)heptane-1,7- | 324.467 | 1.2938 | 47.1 |
| dione | | | |
| 1,9-bis(piperazin-1-yl)nonane-1,9-dione | 324.467 | 1.6968 | 64.68 |
| 1,10-bis(piperazin-1-yl)decane-1,10-dione | 338.494 | 2.1512 | 64.68 |
| 1-(4-methylpiperazin-1-yl)hexan-1-one | 198.309 | 1.8171 | 23.55 |
| 1,10-bis(4-methylpiperazin-1-yl)decane-1,10- | 366.548 | 2.657 | 47.1 |
| dione | | | |
| 1-(piperazin-1-yl)pentan-1-one | 170.255 | 1.1098 | 32.34 |
| 1-(4-hexanoylpiperazin-1-yl)hexan-1-one | 282.426 | 3.6914 | 40.62 |
| 1-(4-heptanoylpiperazin-1-yl)heptan-1-one | 310.48 | 4.6002 | 40.62 |
| 1-(4-octadecanoylpiperazin-1-yl)octadecan-1- | 619.071 | 14.597 | 40.62 |
| one | | | |
| 1-(4-methylpiperazin-1-yl)octadecan-1-one | 366.631 | 7.2699 | 23.55 |
| 1-(4-methylpiperazin-1-yl)heptan-1-one | 212.336 | 2.2715 | 23.55 |
| 1-(4-methylpiperazin-1-yl)octan-1-one | 226.362 | 2.7259 | 23.55 |
| 1-(4-methylpiperazin-1-yl)nonan-1-one | 240.389 | 3.1803 | 23.55 |
| 1-(4-dodecanoylpiperazin-1-yl)dodecan-1-one | 450.749 | 9.1442 | 40.62 |

In another aspect, the first penetration enhancer, the second penetration enhancer, or both can be any suitable piperidine as shown in Table O:

TABLE O
Piperidine Penetration Enhancers

| | | | |
|---|---|---|---|
| 1-[2-(butylsulfanyl)ethyl]piperidine-3-carboxylic acid | 245.386 | 1.0252 | 65.84 |
| 1-[2-(pentylsulfanyl)ethyl]piperidine-3-carboxylic acid | 259.413 | 1.4796 | 65.84 |
| 1-[2-(hexylsulfanyl)ethyl]piperidine-3-carboxylic acid | 273.439 | 1.934 | 65.84 |
| 1-[2-(heptylsulfanyl)ethyl]piperidine-3-carboxylic acid | 287.466 | 2.3884 | 65.84 |
| 1-[2-(octylsulfanyl)ethyl]piperidine-3-carboxylic acid | 301.493 | 2.8428 | 65.84 |
| 1-[2-(nonylsulfanyl)ethyl]piperidine-3-carboxylic acid | 315.52 | 3.2972 | 65.84 |
| 1-[2-(decylsulfanyl)ethyl]piperidine-3-carboxylic acid | 329.547 | 3.7516 | 65.84 |
| 1-[2-(dodecylsulfanyl)ethyl]piperidine-3-carboxylic acid | 357.601 | 4.6604 | 65.84 |
| 1-[2-(tetradecylsulfanyl)ethyl]piperidine-3-carboxylic acid | 385.654 | 5.5692 | 65.84 |
| 1-[2-(hexadecylsulfanyl)ethyl]piperidine-3-carboxylic acid | 413.708 | 6.478 | 65.84 |
| 1-(piperidin-1-yl)dodecan-1-one | 267.455 | 5.5981 | 20.31 |
| 1-dodecylpiperidine | 253.472 | 5.9477 | 3.24 |
| 1-butyl-6-oxopiperidine-2-carboxylic acid | 199.249 | 0.7947 | 57.61 |
| 6-oxo-1-pentylpiperidine-2-carboxylic acid | 213.276 | 1.2491 | 57.61 |
| 1-hexyl-6-oxopiperidine-2-carboxylic acid | 227.303 | 1.7035 | 57.61 |
| 1-heptyl-6-oxopiperidine-2-carboxylic acid | 241.33 | 2.1579 | 57.61 |
| 1-octyl-6-oxopiperidine-2-carboxylic acid | 255.356 | 2.6123 | 57.61 |
| 1-nonyl-6-oxopiperidine-2-carboxylic acid | 269.383 | 3.0667 | 57.61 |
| octyl 2-(2-oxopiperidin-1-yl)acetate | 269.383 | 3.1218 | 46.61 |
| 1-dodecanoylpiperidin-2-one | 281.438 | 5.0319 | 37.38 |
| 1-decyl-6-oxopiperidine-2-carboxylic acid | 283.41 | 3.5211 | 57.61 |
| nonyl 2-(2-oxopiperidin-1-yl)acetate | 283.41 | 3.5762 | 46.61 |
| decyl 2-(2-oxopiperidin-1-yl)acetate | 297.437 | 4.0306 | 46.61 |
| 1-dodecyl-6-oxopiperidine-2-carboxylic acid | 311.464 | 4.4299 | 57.61 |
| undecyl 2-(2-oxopiperidin-1-yl)acetate | 311.464 | 4.485 | 46.61 |
| dodecyl 2-(2-oxopiperidin-1-yl)acetate | 325.491 | 4.9394 | 46.61 |
| 6-oxo-1-tetradecylpiperidine-2-carboxylic acid | 339.518 | 5.3387 | 57.61 |
| tridecyl 2-(2-oxopiperidin-1-yl)acetate | 339.518 | 5.3938 | 46.61 |
| tetradecyl 2-(2-oxopiperidin-1-yl)acetate | 353.545 | 5.8482 | 46.61 |
| 1-hexadecyl-6-oxopiperidine-2-carboxylic acid | 367.572 | 6.2475 | 57.61 |
| 1-butylpiperidin-2-one | 155.24 | 1.7463 | 20.31 |

In another aspect, the first penetration enhancer, the second penetration enhancer, or both can be any suitable pyrrolidine as shown in Table P:

TABLE P
Pryyolidine Penetration Enhancers

| | | | |
|---|---|---|---|
| octyl 1-acetylpyrrolidine-2-carboxylate | 269.383 | 3.3211 | 46.61 |
| decyl 1-acetylpyrrolidine-2-carboxylate | 297.437 | 4.2299 | 46.61 |
| undecyl 1-acetylpyrrolidine-2-carboxylate | 311.464 | 4.6843 | 46.61 |
| dodecyl 1-acetylpyrrolidine-2-carboxylate | 325.491 | 5.1387 | 46.61 |
| 1-dodecylpyrrolidine | 239.445 | 5.6057 | 3.24 |
| 1-(pyrrolidin-1-yl)dodecan-1-one | 253.428 | 5.2561 | 20.31 |
| pentyl 1-acetylpyrrolidine-2-carboxylate | 227.303 | 1.9579 | 46.61 |
| 1-(2,3-dihydroxypropyl)pyrrolidin-2-one | 159.184 | −0.9986 | 60.77 |
| 1-butyl-5-oxopyrrolidine-2-carboxylic acid | 185.222 | 0.4527 | 57.61 |
| 5-oxo-1-pentylpyrrolidine-2-carboxylic acid | 199.249 | 0.9071 | 57.61 |
| 1-hexyl-5-oxopyrrolidine-2-carboxylic acid | 213.276 | 1.3615 | 57.61 |
| 1-heptyl-5-oxopyrrolidine-2-carboxylic acid | 227.303 | 1.8159 | 57.61 |
| 1-octyl-5-oxopyrrolidine-2-carboxylic acid | 241.33 | 2.2703 | 57.61 |
| 1-nonyl-5-oxopyrrolidine-2-carboxylic acid | 255.356 | 2.7247 | 57.61 |
| octyl 2-(2-oxopyrrolidin-1-yl)acetate | 255.356 | 2.7798 | 46.61 |
| 1-decyl-5-oxopyrrolidine-2-carboxylic acid | 269.383 | 3.1791 | 57.61 |
| decyl 5-oxopyrrolidine-2-carboxylate | 269.383 | 3.3541 | 55.4 |
| nonyl 2-(2-oxopyrrolidin-1-yl)acetate | 269.383 | 3.2342 | 46.61 |
| 2-(2-oxopyrrolidin-1-yl)ethyl decanoate | 283.41 | 3.6886 | 46.61 |
| decyl 2-(2-oxopyrrolidin-1-yl)acetate | 283.41 | 3.6886 | 46.61 |
| 1-dodecyl-5-oxopyrrolidine-2-carboxylic acid | 297.437 | 4.0879 | 57.61 |
| undecyl 2-(2-oxopyrrolidin-1-yl)acetate | 297.437 | 4.143 | 46.61 |
| dodecyl 2-(2-oxopyrrolidin-1-yl)acetate | 311.464 | 4.5974 | 46.61 |
| 5-oxo-1-tetradecylpyrrolidine-2-carboxylic acid | 325.491 | 4.9967 | 57.61 |
| tridecyl 2-(2-oxopyrrolidin-1-yl)acetate | 325.491 | 5.0518 | 46.61 |
| tetradecyl 2-(2-oxopyrrolidin-1-yl)acetate | 339.518 | 5.5062 | 46.61 |
| 1-hexadecyl-5-oxopyrrolidine-2-carboxylic acid | 353.545 | 5.9055 | 57.61 |
| 1-dodecyl-5-oxopyrrolidine-3-carboxylic acid | 297.437 | 4.0325 | 57.61 |
| methyl 1-dodecyl-5-oxopyrrolidine-3-carboxylate | 311.464 | 4.4604 | 46.61 |
| 5-methylpyrrolidin-2-one | 99.1325 | 0.161 | 29.1 |
| 1-[2-(decylsulfanyl)ethyl]pyrrolidin-2-one | 285.494 | 4.7769 | 45.61 |
| 5-oxopyrrolidine-2-carboxylic acid | 129.115 | −1.1153 | 66.4 |
| dodecyl 5-oxopyrrolidine-2-carboxylate | 297.437 | 4.2629 | 55.4 |
| 1-ethylpyrrolidin-2-one | 113.159 | 0.4955 | 20.31 |
| 1-octylpyrrolidin-2-one | 197.321 | 3.2219 | 20.31 |
| 1-dodecylpyrrolidin-2-one | 253.428 | 5.0395 | 20.31 |
| 1-(2-hydroxyethyl)pyrrolidin-2-one | 129.158 | −0.4312 | 40.54 |
| 1-butylpyrrolidin-2-one | 141.213 | 1.4043 | 20.31 |
| 1-propylpyrrolidin-2-one | 127.186 | 0.9499 | 20.31 |
| 1-(adamantan-1-yl)pyrrolidin-2-one | 219.327 | 2.1644 | 20.31 |
| 5-oxo-1-phenylpyrrolidine-3-carboxylic acid | 205.212 | 0.5774 | 57.61 |
| 1-[3-(2-oxopyrrolidin-1-yl)butyl]pyrrolidin-2-one | 224.303 | 0.8275 | 40.62 |
| 1-methyl-5-oxopyrrolidine-3-carboxylic acid | 143.141 | −0.9178 | 57.61 |
| 1-[3-(2-oxopyrrolidin-1-yl)propyl]pyrrolidin-2-one | 210.276 | 0.4682 | 40.62 |
| 1-hexylpyrrolidin-2-one | 169.267 | 2.3131 | 20.31 |
| 1,5-dimethylpyrrolidin-2-one | 113.159 | 0.4139 | 20.31 |
| 1-decylpyrrolidin-2-one | 225.374 | 4.1307 | 20.31 |
| methyl 1-methyl-5-oxopyrrolidine-3-carboxylate | 157.168 | −0.4899 | 46.61 |
| pyrrolidin-2-one | 85.1056 | −0.1637 | 29.1 |
| 1-(3-hydroxypropyl)pyrrolidin-2-one | 143.185 | 0.0232 | 40.54 |
| methyl 5-oxo-1-phenylpyrrolidine-3-carboxylate | 219.239 | 1.1953 | 46.61 |
| 1-pentylpyrrolidin-2-one | 155.24 | 1.8587 | 20.31 |
| ethyl 5-oxopyrrolidine-2-carboxylate | 157.168 | −0.2811 | 55.4 |
| 1-dodecanoylpyrrolidin-2-one | 267.411 | 4.6899 | 37.38 |
| 1-methylpyrrolidin-2-one | 99.1325 | 0.0892 | 20.31 |
| 3-dodecyl-1-methylpyrrolidin-2-one | 267.455 | 5.3569 | 20.31 |

In another aspect, the first penetration enhancer, the second penetration enhancer, or both can be any suitable steroid as shown in Table Q:

TABLE Q
Steroid Penetration Enhancers

| | | | |
|---|---|---|---|
| 2-(4-{4,5,7-trihydroxy-9a,11a-dimethyl-hexadecahydro-1H- | 464.62 | 0.1871 | 129.92 |
| cyclopenta[a]phenanthren-1-yl}pentanamido)acetate | | | |
| 2-(4-{7-hydroxy-9a,11a-dimethyl-hexadecahydro-1H- | 483.711 | 1.02 | 112.08 |
| cyclopenta[a]phenanthren-1-yl}pentanamido)ethane-1-sulfonic acid | | | |
| 2-(4-{4,5,7-trihydroxy-9a,11a-dimethyl-hexadecahydro-1H- | 515.709 | 1.2391 | 152.54 |
| cyclopenta[a]phenanthren-1-yl}pentanamido)ethane-1-sulfonic acid | | | |
| 2-(4-{7,11-dihydroxy-9a,11a-dimethyl-hexadecahydro-1H- | 449.629 | 3.1152 | 106.86 |
| cyclopenta[a]phenanthren-1-yl}pentanamido)acetic acid | | | |
| 4-{7-hydroxy-9a,11a-dimethyl-hexadecahydro-1H- | 376.579 | 4.8836 | 57.53 |
| cyclopenta[a]phenanthren-1-yl}pentanoic acid | | | |
| 2-(4-{7-hydroxy-9a,11a-dimethyl-hexadecahydro-1H- | 433.63 | 3.9673 | 86.63 |
| cyclopenta[a]phenanthren-1-yl}pentanamido)acetic acid | | | |
| 2-(4-{4,7,11-trihydroxy-9a,11a-dimethyl-hexadecahydro-1H- | 465.628 | 1.02 | 127.09 |
| cyclopenta[a]phenanthren-1-yl}pentanamido)acetic acid | | | |
| 2-(4-{4,7-dihydroxy-9a,11a-dimethyl-hexadecahydro-1H- | 499.71 | 2.0912 | 132.31 |
| cyclopenta[a]phenanthren-1-yl}pentanamido)ethane-1-sulfonic acid | | | |
| 2-(4-{4,7-dihydroxy-9a,11a-dimethyl-hexadecahydro-1H- | 499.71 | 1.02 | 132.31 |
| cyclopenta[a]phenanthren-1-yl}pentanamido)ethane-1-sulfonic acid | | | |
| 2-(4-{7,11-dihydroxy-9a,11a-dimethyl-hexadecahydro-1H- | 499.71 | 1.02 | 132.31 |
| cyclopenta[a]phenanthren-1-yl}pentanamido)ethane-1-sulfonic acid | | | |
| 2-(4-{4,7-dihydroxy-9a,11a-dimethyl-hexadecahydro-1H- | 449.629 | 3.1152 | 106.86 |
| cyclopenta[a]phenanthren-1-yl}pentanamido)acetic acid | | | |
| 3-{dimethyl[3-(4-{4,7,11-trihydroxy-9a,11a-dimethyl-hexadecahydro-1H- | 614.886 | −2.3377 | 155.37 |
| cyclopenta[a]phenanthren-1-yl}pentanamido)propyl]azaniumyl}propane- | | | |
| 1-sulfonate | | | |
| 12-fluoro-11-hydroxy-8-(2-hydroxyacetyl)-6,6,9,13-tetramethyl-5,7- | 434.502 | 1.332 | 93.06 |
| dioxapentacyclo[10.8.0.0¬≤,,Åπ.0,Å¥,,ÅΠ.0¬π¬≥,¬π,ÅΠ]icosa-14,17- | | | |
| dien-16-one | | | |
| 2-(4-{4,7,11-trihydroxy-9a,11a-dimethyl-hexadecahydro-1H- | 515.709 | 1.2391 | 152.54 |
| cyclopenta[a]phenanthren-1-yl}pentanamido)ethane-1-sulfonic acid | | | |

In another aspect, the first penetration enhancer, the second penetration enhancer, or both can be any suitable succinimide as shown in Table R:

TABLE R
Succinimide Penetration Enhancers

| | | | |
|---|---|---|---|
| 1-(3,7-dimethylocta-2,6-dien-1-yl)pyrrolidine- | 235.326 | 3.3641 | 37.38 |
| 2,5-dione | | | |
| 1-dodecylpyrrolidine-2,5-dione | 267.411 | 4.4733 | 37.38 |
| hexyl 6-(2,5-dioxopyrrolidin-1-yl)hexanoate | 297.393 | 3.1224 | 63.68 |
| heptyl 6-(2,5-dioxopyrrolidin-1-yl)hexanoate | 311.42 | 3.5768 | 63.68 |
| octyl 6-(2,5-dioxopyrrolidin-1-yl)hexanoate | 325.447 | 4.0312 | 63.68 |
| nonyl 6-(2,5-dioxopyrrolidin-1-yl)hexanoate | 339.474 | 4.4856 | 63.68 |
| decyl 6-(2,5-dioxopyrrolidin-1-yl)hexanoate | 353.501 | 4.94 | 63.68 |
| undecyl 6-(2,5-dioxopyrrolidin-1-yl)hexanoate | 367.528 | 5.3944 | 63.68 |
| dodecyl 6-(2,5-dioxopyrrolidin-1-yl)hexanoate | 381.555 | 5.8488 | 63.68 |
| 1-methylpyrrolidine-2,5-dione | 113.116 | −0.477 | 37.38 |

In some embodiments, the first penetration enhancer, the second penetration enhancer, or both can comprise or otherwise act or behave as a secondary active agent. In other embodiments, the topical composition can further comprise a secondary active agent that is different from the first or second penetration enhancer.

In one aspect, the secondary active agent can comprise one or more of: an analgesic, an anti-inflammatory agent, an anti-infective agent, an antioxidant, a catalase, an anti-tumor agent, an anesthetic, an anti-rheumatic agent, a growth factor, a cytokine, an amino acid, a protein, a vaccine, a hormone, a vitamin, the like, or combinations thereof.

In another aspect, the secondary active agent can be present at a concentration of from about 0.0001 wt % to about 50 wt %. In one example, the secondary active agent can be present at a concentration of from about 0.0001 wt % to about 20 wt %. In another example, the secondary active agent can be present at a concentration of from about 0.0001 wt % to about 10 wt %. In yet another example, the secondary active agent can be present at a concentration of from about 0.1 wt % to about 6 wt %.

The secondary active agent can comprise an analgesic that can enhance the cannabinoid receptor modulator (e.g., CBD) relative to a topical composition without the analgesic. In one aspect, the analgesic can be present in the topical composition at a concentration of from about 0.0001 wt % to about 35 wt %. In one example, the analgesic can be present at a concentration of from about 0.5 wt % to about 10 wt %. In another example, the analgesic can be present at a concentration of from about 1 wt % to about 6 wt %. In yet another example, the analgesic can be present at a concentration of from about 0.2 wt % to about 3 wt %. In yet another example, the analgesic can be present at a concentration of from about 0.2 wt % to about 5 wt %. In yet another example, the analgesic can be present at a concentration of from about 2.0 wt % to about 10 wt %.

In one aspect, non-limiting examples of analgesics can include anti-inflammatory agents, such as those listed below, acetaminophen, codeine, dihydrocodeine, tramadol, meperidine, hydrocodone, oxycodone, morphine, fentanyl, hydromorphone, buprenorphine, methadone, diamorphine, pethidine, the like, hydrates thereof, acids thereof, bases thereof, or salts thereof, or combinations thereof.

In some aspects, the analgesic can comprise, but is not limited to, one or more of palmitoylethanolamide, β-caryophyllene, histamine, dihydrochloride, niaouli (Melalenca viridiflora) oil, myrcene, menthol, the like, or combinations thereof.

The secondary active agent can comprise an anti-inflammatory agent that can enhance the cannabinoid receptor modulator (e.g., CBD) relative to a topical composition without the anti-inflammatory agent. In one aspect, the anti-inflammatory agent can be present in the topical composition at a concentration of from about 0.0001 wt % to about 35 wt %. In one example, the anti-inflammatory agent can be present at a concentration of from about 0.5 wt % to about 10 wt %. In another example, the anti-inflammatory agent can be present at a concentration of from about 0.1 wt % to about 3 wt %. In yet another example, the anti-inflammatory agent can be present at a concentration of from about 0.1 wt % to about 2.5 wt %. In yet another example, the anti-inflammatory agent can be present at a concentration of from about 0.2 wt % to about 5 wt %.

In one aspect, non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, durasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof, or combinations thereof.

In another aspect, the anti-inflammatory agent can comprise, but is not limited to, one or more of: palmitoylethanolamide, bisabolol, linalool, myrcene, the like, or combinations thereof.

The secondary active agent can comprise an anti-infective agent, namely an agent that can kill or prevent an infectious organism (e.g., a pathogen) from spreading. In one aspect, the anti-infective agent can be present in the topical composition at a concentration of from about 0.0001 wt % to about 15 wt %. In one example, the anti-infective agent can be present at a concentration of from about 1 wt % to about 6 wt %. In another example, the anti-infective agent can be present at a concentration of from about 0.1 wt % to about 3 wt %. In yet another example, the anti-infective agent can be present at a concentration of from about 0.2 wt % to about 3 wt %.

Thus, anti-infective agents can include antibacterial agents, antifungal agents, antiviral agents, antiprotozoan agents, the like, or combinations thereof. Non-limiting examples can include amebicides such as chloroquine, nitazoxanide, paromomycin, tinidazole, metronidazole, iodoquinole, or the like; aminoglycosides such as tobramycin, gentamicin, amikacin, kanamycin, neomycin, streptomycin, or the like; anthelmintics such as albendazole, ivermectin, praziquantel, pyrantel, mebendazole, miltefosine, niclosamide, piperazine, thiabendazole, or the like; antifungals such as itraconazole, posaconazole, ketoconazole, fluconazole, clotrimazole, isavuconazole, miconazole, voriconazole, echinocandins, terbinafine, griseofulvin, flucytosine, nystatin, amphotericin b, or the like; antimalarials such as chloroquine, quinine, hydroxychloroquine, mefloquine, primaquine, pyrimethamine, halofantrine, doxycycline, or the like; antituberculosis agents such as aminosalicylic acid, bedaquiline, isoniazid, ethambutol, pyrazinamide, ethionamide, rifampin, rifabutin, rifapentine, capreomycin, cycloserine, streptomycin, or the like; antivirals such as amantadine, rimantadine, ritonavir, cobicistat, peginterferon alfa-2a, peginterferon alfa 2b, maraviroc, raltegravir, dolutegravir, elvitegravir, sofosbuvir, enfuvirtide, fomivirsen, foscarnet, oseltamivir, zanamivir, peramivir, etravirine, efavirenz, nevirapine, delavirdine, rilpivirine, daclatasvir, adefovir, entecavir, telbivudine, didanosine, tenofovir, abacavir, lamivudine, zidovudine, stavudine, emtricitabine, zalcitabine, boceprevir, simeprevir, fosamprenavir, lopinavir, darunavir, telaprevir, ritonavir, tipranavir, atazanavir, nelfinavir, amprenavir, indinavir, saquinavir, ganciclovir, valacyclovir, famciclovir, acyclovir, valganciclovir, ribavirin, cidofovir, or the like; carbapenems such as doripenem, meropenem, cilastatin, ertapenem, or the like; cephalosporins such as avibactam, ceftolozane, ceftazidime, tazobactam, cefadroxil, cephalexin, cefazolin, ceftaroline, loracarbef, cefotetan, cefuroxime, cefprozil, cefaclor, cefoxitin, ceftibuten, cefotaxime, ceftriaxone, cefpodoxime, cefixime, cefdinir, defditoren, ceftazidime, ceftizoxime, cefepime, or the like; glycopeptide antibiotics such as vancomycin, dalbavancin, oritavancin, telavancin, or the like; glycocyclines such as tigecycline, or the like; leprostatics such as thalidomide, dapsone, clofazimine, or the like; lincomycin, or the like: clindamycin, or the like; ketolides such as telithromycin, or the like; macrolides such as azithromycin, fidaxomicin, erythromycin, clarithromycin, or the like; antibiotics such as aztreonam, daptomycin, chloramphenicol, colistimethate, fosfomycin, rifaximin, metronidazole, sulfamethoxazole, atovaquone, bacitracin, dalfopristin, erythromycin, furazolidone, pentamidine, polymyxin b, spectinomycin, trimetrexate, linezolid, tedizolid, penicillins (e.g. ampicillin, amoxicillin, carbenicillin, piperacillin, ticarcillin, nafcillin, dicloxacillin, cloxacillin, oxacillin, or the like), quinolones (e.g. lomefloxacin, norfloxacin, ofloxacin, gatifloxacin, moxifloxacin, ciprofloxacin, levofloxacin, gemifloxacin, cinoxacin, nalidixic acid, sparfloxacin, or the like), sulfonamides (e.g. sulfamethoxazole, sulfadiazine, sulfisoxazole, or the like), tetracyclines (e.g. tetracycline, demeclocycline, doxycycline, minocycline, or the like), or the like; urinary anti-infectives such as methenamine, methylene blue, fosfomycin, nitrofurantoin, trimethoprim, cinoxacin, nalidixic acid, oxytetracycline, or the like; hydrates thereof, acids thereof, bases thereof, salts thereof, or combinations of any of such anti-infective agents.

In some aspects, the anti-infective agent can comprise, but is not limited to, one or more of bisabolol, β-caryophyllene, niaouli oil, the like, or combinations thereof.

In some embodiments, the secondary active agent can comprise an antioxidant. In one aspect, the antioxidant can be present in the topical composition at a concentration of from about 0.0001 wt % to about 10 wt %. In one example, the antioxidant can be present in the topical composition at a concentration of from about 0.1 wt % to about 2 wt %. In another example, the antioxidant can be present in the topical composition at a concentration of from about 0.1 wt % to about 1 wt %.

Various antioxidants can be used including, but not limited to: N-acetyl-cysteine, hydroxytyrosol (HXT), reduced glutathione (rGSH), catalase, Vitamin A, Vitamin C, Vitamin E, coenzyme Q10, managanese, iodide, melatonin, alpha-carotene, astaxanthin, beta-carotene, canthaxanthin, cryptoxanthin, lutein, lycopene, zeaxanthin, apigenin, luteolin, tangeritin, isorhamnetin, kaempferol, myricetin, proanthocyanidins, quercetin, eriodyctiol, hesperetin, naringenin, catechin, gallocatechin, epicatechin, epigallocatechin, theaflavin, thearubigins, daidzein, genistein, glycitein, resveratrol, pterostilbene, cyanidin, delphinidin, malvidin, pelargonidin, peonidin, petunidin, chicoric acid, cholorogenic acid, cinnamic acid, ellagic acid, ellagitannins, gallic acid, gallotannins, rosmarinic acid, salicylic acid, acid, curcumin, flavonolignans, xanthones, eugenol, capsaicin, bilirubin, citric acid, oxalic acid, phytic acid, R-alpha-Lipoic acid, the like, and combinations thereof.

In some aspects, the antioxidant can comprise, but is not limited to, one or more of: D-limonene, terpinolene, the like, or combinations thereof.

In some examples, the secondary active agent can also include any suitable antitumor agent. Non-limiting examples of antitumor agents can include angiogenesis inhibitors such as angiostatin k1-3, angiostatin k1-5, DL-α-difluoromethylornithine, endostatin, fumagillin, genistein, minocycline, staurosporine, (+/−)-thalidomide, or the like; DNA intercalators such as bleomycin, carboplatin, carmustine, chlorambucil, cisplatin, cyclophosphamide, cis-diammineplatinum(II) dichloride, melphalan, mitoxantrone, oxaliplatin, or the like; DNA synthesis inhibitors such as (+/−)-amethopterin, 3-amino-1,2,4-benzotraizine-1,4-dioxide, aminopterin, cytosine β-D-arabinofuranoside, 5-fluoro-5′-deoxyuridine, 5-fluorouracil, ganciclovir, hydroxyurea, mitomycin C, or the like; transcription regulators such as actinomycin D, daunorubicin, doxorubicin, homoharringtonine, idarubicin, or the like; enzyme inhibitors such as S(+)-camptothecin, curcumin, (−)-deguelin, 5,6-dichlorobenzimidazole 1-β-D-ribofuranoside, etoposide, formestane, fostriecin, hispidin, 2-imino-1-imidazolidineacetic acid, mevinolin, trichostatin A, tyrphostin AG 34, tyrphostin AG 879, or the like; gene regulation agents such as 5-aza-2′-deoxycytidine, 5-azacytidine, cholecalciferol, 4-hydroxytamoxifen, melatonin, mifepristone, raloxifene, vitamin A aldehyde, vitamin A acid, vitamin A, 9-cis-retinoic acid, 13-cis-retinoic acid, tamoxifen, troglitazone, or the like, microtubule inhibitors such as colchicine, docetaxel, dolastatin 15, etoposide, irinotecan, nocodazole, paclitaxel, podophyllotoxin, rhizoxin, vinblastine, vincristine, vindesine, vinorelbine, or the like; other antitumor agents such as 17-(allylamino)-17-demethoxygeldanamycin, 4-amino-1,8-naphthalimide, apigenin, brefeldin A, cimetidine, dichloromethylene-diphosphonic acid, leuprolide, luteinizing hormone-releasing hormone, pifithrin-α, rapamycin, sex hormone-binding globulin, thapsigargin, bikunin, ifosfamide, temozolomide, capecitabine, methotrexate, gemcitabine, pemetrexed, mitomycin, epirubicin, bevacizumab, cetuximab, gefitinib, imatinib, trastuzamab, denosumab, rituximab, sunitinib, zoledronate, abiraterone, anastrozole, bicalutamide, exemestane, goserelin, medroxyprogesterone, octreotide, tamoxifen, bendamustine, lomustine, procarbazine, streptozocin, fludarabine, raltitrexed, mitoxantrone, eribulin, topotecan, afatinib, aflibercept, BCG, crizotinib, dabrafenib, interferon, ipilimumab, lapatinib, nivolumab, panitumumab, pembrolizumab, pertuzumab, sorafenib, trastuzumab emtansine, temsirolimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, or the like, hydrates thereof, acids thereof, bases thereof, salts thereof, or combinations of any of such antitumor agents.

In some examples, the secondary active agent can also include any suitable anesthetic. Non-limiting examples of anesthetics can include articaine, bupivacaine, cinchocaine, etidocaine, levobupivacaine, lidocaine, mepivacaine, prilocaine, ropivacaine, trimecaine, the like, or combinations thereof.

In some examples, the secondary active agent can also include any suitable anti-rheumatic agent. Non-limiting examples of anti-rheumatic agents can include methotrexate, sulfasalazine, chloroquine, hydroxychloroquine, leflunomide, azathioprine, cyclosporine, minocycline, abatacept, rituximab, tocilizumab, anakinra, adalimumab, etanercept, infliximab, cetolizumab, golimumab, D-penicillamine, auranofin, the like, hydrates thereof, acids thereof, bases thereof, or salts thereof, or combinations thereof.

In some examples, the secondary active agent can also include any suitable growth factor. Non-limiting examples of supplementary growth factors can include platelet-derived growth factor (PDGF), epidermal growth factor (EGF), transforming growth factor-alpha (TGF-α), fibroblast growth factor (FGF), nerve growth factor (NGF), erythropoietin, transforming growth factor-beta (TGF-β), insulin-like growth factor-1 (IGF-1), insulin-like growth factor-2 (IGF-2), the like, or combinations thereof.

In some examples, the secondary active agent can also include any suitable supplementary cytokine. Non-limiting examples of supplementary cytokines can include interleukins, lymphokines, monokines, interferons, colony stimulating factors, chemokines, the like, or combinations thereof.

In some examples, the secondary active agent can also include any suitable amino acid. Non-limiting examples can include arginine, histidine, isoleucine, leucine, lysine, methionine, phenylalanine, tryptophan, valine, acetyl-L-carinitine arginate, alpha-aminoadipic acid, alpha-amino-N-butyric acid, beta-alanine, beta-amino-isobutyric acid, carnosine, citruline, gamma-amino butyric acid, hydroxyproline, 1-methylhistidine, 3-methylhistidine, N-acytyl-L-cysteine, ornithine, para-aminobenzoic acid, phosphoserine, phosphoethanolamine, taurine, the like, isomers thereof, hydrates thereof, salts thereof, acids thereof, bases thereof, or any combinations thereof.

In some examples, the secondary active agent can also include any suitable protein. Non-limiting examples can include cytokines and/or growth factors, such as those listed above, as well as antibodies, Fc-fusion proteins, anticoagulants, blood factors, bone morphogenetic proteins, engineered protein scaffolds, enzymes, hormones, thrombolytics, the like, or combinations thereof.

In some examples, the secondary active agent can also include a vaccine. Non-limiting examples of vaccines can include adenovirus vaccine, coronavirus, coxsackie B vaccine, cytomegalovirus vaccine, dengue vaccine. Eastern equine encephalitis vaccine, ebola vaccine, enterovirus vaccine, Epstein-barr vaccine, hepatitis A vaccine, hepatitis B vaccine, hepatitis C vaccine, hepatitis E vaccine, HIV vaccine, human papillomavirus vaccine, HTLV-1 T-lymphotrophic vaccine, influenza vaccine. Japanese encephalitis vaccine, Marburg vaccine, measles vaccine, mumps vaccine, norovirus vaccine, polio vaccine, rabies vaccine, respiratory syncytial virus (RSV) vaccine, rotavirus vaccine, rubella vaccine, severe acute respiratory syndrome (SARS) vaccine, varicella vaccine, smallpox vaccine. West Nile virus vaccine, yellow fever vaccine, anthrax vaccine, DPT vaccine, Q fever vaccine, Hib vaccine, tuberculosis vaccine, meningococcal vaccine, typhoid vaccine, pneumococcal vaccine, cholera vaccine, caries vaccine, ehrlichiosis vaccine, leprosy vaccine, lyme disease vaccine, Staphylococcus aureus vaccine, Streptococcus pyogenes vaccine, syphilis vaccine, tularemia vaccine, Yersinia pestis vaccine, the like, or combinations thereof.

In some examples, the secondary active agent can also include a hormone. Non-limiting examples of hormones can include progestogens, androgens, estrogens, somatostatins, growth hormones, thyroid hormones, glucocorticoids, the like, or combinations thereof.

In some examples, the secondary active agent can also include a vitamin. Non-limiting vitamins can include vitamin A, vitamin B1, vitamin B2, vitamin B6, vitamin B12, vitamin C, vitamin D, vitamin E, vitamin H, vitamin K, folic acid, the like, or combinations thereof.

The carrier can include a number of ingredients that contribute to or otherwise achieve the increased or enhanced bioavailability as compared to a composition of a cannabinoid receptor modulator without the combination of first and second penetration enhancers. Such ingredients can generally include solubilizers, solvents, water, cosolvents, and additives, surfactants, including hydrophilic, hydrophobic, and amphoteric surfactants, as well as oils, fats, carotenoids, nanocarotinoids, among others.

A wide variety of solubilizers can be used in the carrier of the present compositions. Examples of suitable solubilizers can comprise but are not limited to: (i) tocopherol or its derivatives: (ii) fatty acid or its salts; (iii) glyceryl fatty acid esters; (iv) PEG glycerides of fatty acid esters; (v) polyglycerol fatty acid esters; (vi) triglycerides; (vii) hydrogenated polyoxyl vegetable oils or glycerides; (viii) propylene glycol fatty acid esters; (ix) vegetable oils; (x) sterols or its derivatives, the like, or combinations thereof.

In one aspect, vitamin E or its derivatives can comprise, but are not limited to: alpha-tocopherol, beta-tocopherol, gamma-tocopherol, delta-tocopherol, tocopherol acetate, tocopherol linoleate, tocopherol succinate, tocotrienols (alpha-, beta-, gamma-, or delta-), tocofersolan (PEG derivatives of alpha-tocopherol), the like, or combinations thereof.

In another aspect, fatty acid or its salts can comprise, but not limited to: octanoic acid, capric acid, lauric acid, myristic acid, palmitic acid, stearic acid, oleic acid, linoleic acid, linoelaidic acid, sodium caproate, sodium caprylate, sodium laurate, sodium myristate, sodium palmitate, sodium oleate, sodium stearate, sodium lauryl sulfate, sodium lauryl sarcosinate, sodium dioctyl sulfosuccinate, sodium cholate, sodium taurocholate, the like, or combinations thereof.

In another aspect, glyceryl fatty acid esters can comprise, but are not limited to: glyceryl monooleate, glyceryl monoleate/linoleate, glyceryl monolinoleate, glyceryl ricinoloeate, glyceryl monolaurate, glyceryl monopalmitate, glyceryl monostearate, glyceryl mono-/di-oleate, glyceryl palmtate/stearate, glyceryl acetate, glyceryl laurate, glyceryl citrate/lactate/oleate/linoleate, glyceryl caprylate, glyceryl caprylate/caprate, glyceryl dicaprylate/dicaprate, mono-/di-acetylated monoglycerides, glyceryl monostearate, glyceryl dilaurate, glyceryl dioleate, the like, or combinations thereof.

In another aspect, PEG glycerides of fatty acid esters can comprise, but are not limited to: PEG fatty acid monoesters, PEG glycerol fatty acid esters, PEG fatty acid diesters, PEG fatty acid mono-/di-ester mixtures, PEG triglycerides of fatty acid esters, the like, or combinations thereof. PEG glycerol fatty acid esters can comprise, but are not limited to: PEG glyceryl laurate, PEG glyceryl laurate, PEG glyceryl caprylate, PEG glyceryl caprate, PEG glyceryl oleate, PEG glyceryl mono-di-fatty acid ester mixtures, the like, or combinations thereof. PEG fatty acid monoesters can comprise, but are not limited to: esters of caprylic acid, capric acid, lauric acid, oleic acid, and stearic acid, the like, or combinations thereof. Examples of the PEG fatty acid monoesters can include PEG (1-100, 200, 300, 400) monocaprylate, PEG (1-100, 200, 300, 400) monocaprate, PEG (1-100, 200, 300, 400) monolaurate, PEG (1-100, 200, 300, 400) monooleate, PEG (1-100, 200, 300, 400) monopalmitate, PEG (1-100, 200, 300, 400) monostearate, and PEG (1-100, 200, 300, 400) monococoate, the like, or combinations thereof. PEG fatty acid diesters can comprise, but are not limited to, PEG (4-32) dicaprylate, PEG (4-32) dicaprate, PEG (4-32) dilaurate, PEG (4-32) dioleate, PEG (4-32) distearate, and PEG (4-32) dipalmitate, the like, or combinations thereof. PEG fatty acid mono-/di-ester mixtures can comprise, but are not limited to: PEG caprylate/caprate, PEG mono-/di-caprylate, PEG mono-/di-caprate, PEG mono-/di-laurate, PEG mono-/di-oleate, and PEG mono-/di-stearate the like, or combinations thereof. PEG triglycerides of fatty acid esters can comprise, but are not limited to: lauroyl polyoxylglycerides, stearoyl polyoxylglycerides, oleoyl polyoxyl glycerides, linoleoyl polyoxyl glycerides, lauroyl polyoxyl glycerides, caprylocaproyl polyoxyl glycerides, and behenoyl polyoxylglycerides the like, or combinations thereof.

In yet another aspect, polyglycerol fatty acid esters can comprise, but are not limited to: polyglyceryl (2, 3, 4, 6, 10) oleate, polyglyceryl (2, 3, 4, 6, 10) dioleate, polyglyceryl (2, 3, 4, 6, 10) trioleate, polyglyceryl (2, 3, 4, 6, 10) laurate, polyglyceryl (2, 3, 4, 6, 10) dilaurate, polyglycerl (2, 3, 4, 6, 10) trilaurate, polyglyceryl (2, 3, 4, 6, 10) stearate, polyglyceryl (2, 3, 4, 6, 10) distearate, polyglyceryl (2, 3, 4, 6, 10) tristearate, polyglyceryl (2, 3, 4, 6, 10) mono-/di-oleate, polyglyceryl (3,6,10) caprate, polyglyceryl (3,6,10) dicaprate, polyglyceryl (3,6,10) tricaprate, polyglyceryl (3,6,10) caprylate, polyglyceryl (3,6,10) dicaprylate, polyglyceryl (3,6,10) tricaprylate, polyglyceryl (3,6,10) polystearate, polyglyceryl (3,6,10) polyoleate, polyglyceryl (3,6,10) mono-/di-oleate, polyglyceryl (3,6,10) caprylate, polyglyceryl (3,6,10) polycaprylate, polyglyceryl (3,6,10) caprate, polyglyceryl (3,6,10) polycaprate, and polyglyceryl (3,6,10) caprylate/caprate, the like, or combinations thereof.

In another aspect, triglycerides can comprise, but are not limited to: glyceryl tricaprylate, glyceryl tricaprate, glyceryl tricaprylate/tricaprate, glyceryl tricaprylate/tricaprate/trisuccinate, glyceryl trioleate, glyceryl tristearate, glyceryl trilaurate, medium chain natural oils, the like, or combinations thereof.

In yet another aspect, hydrogenated polyoxyl vegetable oils or glycerides can comprise, but are not limited to: castor oil or hydrogenated castor oil, or an edible oil (e.g. vegetable oil) such as corn oil, olive oil, peanut oil, palm kernel oil, apricot kernel oil, peppermint oil, coconut oil, sunflower seed oil, or almond oil, the like, or combinations thereof, canola oil, cotton oil, palmolein, sunflower oil, corn oil, rapeseed oil, grape seeds oil, hemp oil, pomegranate oil, avocado oil, peppermint oil and medium chain triglycerides. The polyoxyl group can include glycerol, propylene glycol, ethylene glycol, polyethylene glycol, sorbitol, pentaerythritol, the like, or combinations thereof. c Examples of hydrogenated polyoxyl vegetable oils or glycerides can comprise, but are not limited to: PEG-35 castor oil (Incrocas-35, Koliphor EL), PEG-40 hydrogenated castor oil (Koliphor RH 40), PEG-25 trioleate (TAGATRTO), PEG-60 cornglycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylickcapric glycerides (Labrasol), PEG-6 caprylic/capric glycerides (Softigen 767), PEG-5 hydrogenated castor oil, PEG-7 hydrogenated castor oil, PEG-9 hydrogenated castor oil, PEG-6 corn oil (Labrafil M. 2125 CS), PEG-6 almond oil (Labrafil M 1966 CS), PEG-6 apricot kernel oil (Labrafil M 1944CS), PEG-6 olive oil (Labrafil M 1980 CS), PEG-6 peanut oil (Labrafil M 1969 CS), PEG-6 hydrogenated palm kernel oil (Labrafil M2130 BS), PEG-6 palm kernel oil (Labrafil M 2130 CS), PEG-6 triolein (Labrafil M 2735 CS), PEG-8 corn oil (Labrafil WL 2609 BS), PEG-20 corn glycerides (Crovol M40), and PEG-20 almond glycerides (Crovol A40), the like, or combinations thereof.

The oil may be present in the formulation, according to some embodiments, at an amount of between about 0.5 and 20 wt %. According to other embodiments, the oil is present in the formulation at an amount of between about 1 and 10 wt %.

In one aspect, propylene glycol fatty acid esters can comprise, but are not limited to: propylene glycol monolaurate (Lauroglycol FCC), propylene glycol ricinoleate (Propymuls), propylene glycol monooleate (Myverol P-06), propylene glycol dicaprylate/dicaprate (Captex 200), and propylene glycol dioctanoate (Captex 800), propylene glycol monocaprylate (Capryol 90, Nikkol Sefsol 218), propylene glycol myristate, propylene glycol monostearate, propylene glycol ricinolate, propylene glycol isostearate, propylene glycol caprylate/caprate, propylene glycol dioleate, propylene glycol distearate, propylene glycol dilaurate, propylene glycol dicaprylate, and propylene glycol dicaprate, the like, or combinations thereof.

In another aspect, vegetable oils can comprise, but are not limited to, corn oil, olive oil, peanut oil, coconut oil, peppermint oil, sunflower seed oil, castor oil, safflower oil, borage oil, cottonseed oil, soybean oil, palm kernel oil, apricot kernel oil, almond oil, the like, or combinations thereof.

In one aspect, sterols or its derivatives can comprise, but are not limited to: cholesterol, sitosterol, lanosterol, phytosterol, its PEG derivatives, the like, or combinations thereof.

In one embodiment, an additive can be substance that can be added to the pharmaceutical formulation to enhance the solubilization, separation, or dispersion of the particles, or to enhance the dissolution and further absorption of the particles into the body. As used herein an additive can be a lipophilic additive when it has an HLB value of or less, or a hydrophilic additive when it has an HLB value of greater than 10.

In one aspect, the pharmaceutically acceptable carrier can comprise a hydrophilic additive, a lipophilic additive, or a combination thereof. In one aspect, lipophilic additives can comprise, but are not limited to: mono-, di-glycerides of fatty acids, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g. Labrafil M 2125 CS), PEG-6 almond oil (e.g. Labrafil M 1966 CS), PEG-6 apricot kernel oil (e.g. Labrafil M 1944 CS), PEG-6 olive oil (e.g. Labrafil M 1980 CS), PEG-6 peanut oil (e.g. Labrafil M 1969 CS), PEG-6 hydrogenated palm kernel oil (e.g. Labrafil M 2130 BS), PEG-6 palm kernel oil (e.g. Labrafil M 2130 CS). PEG-6 triolein (e.g. Labrafil M 2735 CS), PEG-8 corn oil (e.g. Labrafil WL 2609 BS). PEG-20 corn glycerides (e.g. Crovol M40), PEG-20 almond glycerides (e.g. Crovol A40), lipophilic polyoxyethylene-polyoxypropylene block co-polymers (e.g. Pluronic L92, L101, L121 etc.), propylene glycol fatty acid esters, such as propylene glycol monolaurate (e.g. Lauroglycol FCC), propylene glycol ricinoleate (e.g. Propymuls), propylene glycol monooleate (e.g. Myverol P-06), propylene glycol dicaprylate/dicaprate (e.g. Captex® 200), and propylene glycol dioctanoate (e.g. Captex® 800), propylene glycol mono-caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof. In some cases, a solubilizer for the compositions and oral dosage forms can be a lipophilic surfactant.

The pharmaceutically acceptable carrier can also comprise a hydrophilic additive. In one aspect, the hydrophilic additive can comprise non-ionic surfactants, ionic surfactants, zwitterionic surfactants, the like, or combinations thereof. Suitable Hydrophilic surfactants can include, but are not limited to: alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono- and di-ester mixtures; polysorbates, polyethylene glycol derivatives of tocopherol, the like, or combinations thereof. Two or more hydrophilic additives from the same or different classes can be referred to as the hydrophilic surfactant unless explicitly specified. In one aspect, non-limiting examples of hydrophilic surfactants can comprise PEG-8 caprylic/capric glycerides, lauroyl macrogol-32 glyceride, stearoyl macrogol glyceride, PEG-40 hydrogenated castor oil. PEG-35 castor oil, sodium lauryl sulfate, sodium dioctyl sulfosuccinate, polyethylene glycol fatty acids mono- and di-ester mixtures, polysorbate 80, polysorbate 20, polyethylene glycol 1000 tocopherol succinate, phytosterols, phytosterol fatty acid esters, the like, or combinations thereof.

In yet another aspect, additives can comprise sterols and derivatives of sterols. In various aspects, these additional agents can be hydrophilic or lipophilic. Examples of hydrophilic sterols include but are not limited to: lanosterol PEG-24 cholesterol ether (e.g. Solulan C-24, Amerchol), PEG-30 soya sterol (e.g. Nikkol BPS-30, from Nikko), PEG-25 phyto sterol (e.g. Nikkol BPSH-25 from Nikko), PEG-30 cholestanol (e.g. Nikkol DHC, from Nikko). Examples of Lipophilic Sterol Surfactants are Cholesterol, sitosterol, Phytosterol (e.g. GENEROL series from Henkel), PEG-5 soya sterol (e.g. Nikkol BPS-S, from Nikko), PEG-10 soya sterol (e.g. Nikkol BPS-10 from Nikko), PEG-20 soya sterol (e.g. Nikkol BPS-20 from Nikko), the like, or combinations thereof.

In another aspect, the oral compositions can further comprise a polymeric release modifier. The polymeric release modifier can comprise, but is not limited to: celluloses, such as hydroxypropyl celluloses low molecular weight, low viscosity types (e.g. Methocel E5, E6, E10 E15, LV100 etc. grades), hydroxypropyl celluloses having higher molecular weight, medium to high viscosity (e.g. Methocel K4M, K15M, K100M etc), polyvinylpyrrolidones (e.g. Kollidon k17, K30 etc), polyvinyl acetates, hydroxypropyl methylcellulose (HPMC), hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, Sodium croscarmellose, carrageenan, hydroxyethyl cellulose (HEC), carboxymethyl cellulose (CMC) and carbox cellulose (CEC), sodium alginate, polycarbophil, gelatin, Xanthan gum, and sodium starch glycolate, the like, or combinations thereof.

The pharmaceutically acceptable carrier can be combined with additives in various amount ranges. For example, the lipophilic additive and hydrophilic additive can be present in amounts such that the ratio in wt % of lipophilic additive to amount of hydrophilic additive is from 1:10 to 10:1. In some embodiments, the amount can be a ratio of than 2:1 to 1:2. In another aspect the amount can be a ratio of 1:5 to 5:1.

In certain examples, the hydrophilic additive can make up about 1% to about 99% w/w, about 2% to about 80% w/w, about 2% to about 50% w/w, or about 10% to about 40% w/w of any pharmaceutical composition described herein. In some examples, the lipophilic additive can make up about 1% w/w to about 99% w/w, about 2% to about 80% w/w, about 10% w/w to about 80% w/w, about 30% w/w, to about 80% w/w, or about 40% to about 80% w/w of any pharmaceutical composition described herein.

Co-solvents can fully or partially solubilize cannabinoid receptor modulators when presented in an effective amount. Examples of suitable co-solvents can comprise without limitation: alcohols and polyols, such as ethanol, propanol, isopropanol, butanol, benzyl alcohol, ethylene glycol, propylene glycol, butanediols, glycerin or its derivatives thereof, glycerol, diglycerol, polyglycerol, pentaerythritol, sorbitol, mannitol, transcutol, dimethyl isosorbide, triacetin, trimethyl citrate, polyethylene glycol, polypropylene glycol, polyvinylalcohol, hydroxypropyl methylcellulose and other cellulose derivatives, cyclodextrins or its derivatives, the like, or combinations thereof.

### Formulations and Dosage Forms

In some embodiments, topical compositions can be formulated as one of: a solution, a suspension, a liquid, a semi-liquid, an emulsion, a gel, a hydrogel, a thermo-responsive gel, a cream, a lotion, an ointment, a liniment, a paste, a mousse, an aerosol, a spray, a wax, a balm, a suppository, an adhesive, an erodible matrix, a liquid reservoir, a patch, a powder, a compressed powder, the like, or a combination thereof. The dosage forms of the current disclosure can comprise a transdermal dosage form, a topical dosage form, a transmucosal dosage form, the like, or combinations thereof.

In one aspect, the pharmaceutically acceptable carrier can comprise, but is not limited to, one or more of: an emulsifier, a thickener, an emollient, a preservative, a solubilizing agent, a tonicity agent, a buffer, a pH adjuster, water, the like, or combinations thereof.

In one aspect, the emulsifier can be present in the topical composition at a concentration of from about 0.0001 wt % to about 50 wt %. In one example, the emulsifier can be present in the topical composition at a concentration of from about 10 wt % to about 30 wt %. Non-limiting examples of emulsifiers can include water, ethanol, propylene glycol, ethylene glycol, glycerin, polyethylene glycol, banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof. In another aspect, the emulsifier can comprise, but is not limited to, one or more of: octanoic acid triglyceride, decanoic acid triglyceride, cetearyl olivate, sorbitan olivate, sorbitan gum powder, biopolysan 220B, and combinations thereof.

In one aspect, the thickener can be present in the topical composition at a concentration of from about 0.0001 wt % to about 50 wt %. In one example, the thickener can be present in the topical composition at a concentration of from about 1 wt % to about 10 wt %. The thickening agent can comprise, but is not limited to, polyacrylic acids (e.g., Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof.

In another aspect, the thickener can be a polymer. In one example, the thickener can comprise, but is not limited to, one or more of: cetearyl olivate, sorbitan olivate, polyacrylate crosspolymer-6, the like, or combinations thereof. Non-limiting examples of a polymer having thickening properties can include, but are not limited to, a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C₁₀-C₃₀ alkyl acrylate crosspolymers, and combinations thereof.

In one aspect, the emollient can be present in the topical composition at a concentration of from about 1 wt % to about 50 wt %. In one example, the emollient can be present in the topical composition at a concentration of from about 4 wt % to about 20 wt %. Non-limiting examples of emollients can include, but are not limited to, aloe vera, lanolin, urea, petrolatum, shea butter, cocoa butter, mineral oil, paraffin, beeswax, squalene, jojoba oil, coconut oil, sesame oil, almond oil, cetyl alcohol, stearyl alcohol, olive oil, oleic acid, triethylhexanoin, glycerol, sorbitol, propylene glycol, cyclomethicone, dimethicone, the like, or combinations thereof. In one example, the emollient can comprise, but is not limited to, one or more of: cetearyl olivate, sorbitan olivate, biopolysan 220B, Helianthus annus seed oil, laurel laurate, cetyl palmitate, the like, or combinations thereof.

In one aspect, the preservative can be present in the topical composition at a concentration of from about 0.0001 wt % to about 50 wt %. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, anisic acid, dehyroacetic acid, glyceryl caprylate, caprylhydroxamic acid, tocopherols, the like, or combinations thereof. In one aspect, the preservative can comprise, but is not limited to, biopolysan 220B.

In one aspect, the topical composition can have a tonicity of from about 270 milliosmoles/liter (mOsm/L) to about 330 mOsm/L. In another aspect, the tonicity of the composition can be from about 250 to about 350. In another aspect, the tonicity of the composition can be from about 277 to about 310 mOsm/L. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Trode's BSS, and BSS Plus, the like, or combinations thereof. The tonicity agent can be used to provide an appropriate tonicity of the topical composition.

The pH of the topical composition can be from about 5 to about 9, or from about 6 to about 8. In another example, the pH of the therapeutic composition can be from about 5 to about 6. In another example, the topical composition can have a pH of from about 6.8 to about 7.4. In one aspect, the topical composition can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof.

The topical composition can have any suitable viscosity for applying a topical substance. In one example, the topical composition can have a viscosity of from about 1 cP to about 1000 cP. In one example, the topical composition can have a viscosity of from about 1 cP to about 15 cP. In another example, the topical composition can have a viscosity of from about 20 cP to about 100 cP. In one more example, the topical composition can have a viscosity of from about 300 cP to about 1000 cP.

In a further embodiment, a transdermal dosage form for administration of a cannabinoid receptor modulator (e.g., CBD) can include a carrier that is suitable for transdermal administration. The carrier can include one or more of water, a polymer having surfactant properties, a polymer having thickening properties, or a solvent. The transdermal dosage form can comprise penetration enhancers in addition to the combination of first and second penetration enhancers including, but not limited to, one or more of: ethanol, propylene glycol, oleic acid and other fay acids, azone, terpenes, terpenoids, bile acids, isopropyl myristate and other fatty esters, dimethyl sulphoxides, N-methyl-2-pyrrolidone and other pyrrolidones, other penetration enhancers disclosed herein, the like, or combinations thereof.

In some examples, the transdermal dosage form can include a polymer having surfactant or emulsifying properties. Non-limiting examples of a polymer having surfactant or emulsifying properties can include, but are not limited to, hydrophobically modified polyacrylic acid commercially under the tradename Pemulen™ TR-1 and TR-2 by Lubrizol Corp., water-soluble or water-swellable copolymers based on acrylamidoalkyl sulfonic acid and cyclic N-vinylcarboxamides commercially available under the tradename Aristoflex® AVC by Clariant Corporation; water-soluble or water-swellable copolymers based on acrylamidoalkyl sulfonic acid and hydrophobically modified methacrylic acid commercially available under the tradename Aristoflex® HMB by Clariant Corporation and a homopolymer of acrylamidoalkyl sulfonic acid commercially available under the tradename GranthixAPP by Grant Industries, Inc.

Other materials that may be suitable polymeric surfactants can include ethylene oxide/propylene oxide block copolymers, sold under the trade name PLURONIC®, available from BASF Corporation of Parsippany, NJ., modified cellulose polymers such as those modified cellulose polymers described by the trade name KLUCEL®, available from Hercules Corporation of Wilmington, DE. Some examples include hydrophobically modified polyacrylic acid, acrylamidoalkyl sulfonic acid, cyclic N-vinylcarboxamides, acrylamidoalkyl sulfonic acid, hydrophobically modified methacrylic acid, a homopolymer of acrylamidoalkyl sulfonic acid, or combinations thereof as polymeric emulsifiers; and monomeric anionic surfactants, monomeric amphoteric surfactants, or combinations thereof as foaming agents. Some more examples are compositions that include hydrophobically modified polyacrylic acid; water-soluble or water-swellable copolymers based on acrylamidoalkyl sulfonic acid, cyclic N-vinylcarboxamides; water-soluble or water-swellable copolymers based on acrylamidoalkyl sulfonic acid, hydrophobically modified methacrylic acid: a homopolymer of acrylamidoalkyl sulfonic acid, or combinations thereof as polymeric emulsifiers, and include a betaine as the foaming surfactant. Some examples are compositions that include copolymers based on acrylamidoalkylsulfonic acids and cyclic N-vinylcarboxamides and/or linear N-vinylcarboxamides (e.g., Aristoflex® AVC and Aristoflex®, HMB from Clariant Corporation) as polymeric emulsifiers and a betaine as foaming surfactant.

In some examples, the transdermal dosage form can include a polymer having thickening properties. Non-limiting examples of a polymer having thickening properties can include, but are not limited to, a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C₁₀-C₃₀ alkyl acrylate crosspolymers, and combinations thereof.

In a further embodiment, a transmucosal dosage form for administration of a cannabinoid receptor modulator (e.g., CBD or β-caryophyllene) can include a carrier that is suitable for transmucosal administration. The dosage form can further include one or more of an adhesive layer, a bio-erodible matrix, or a solution. The dosage form can be suitable for nasal, vaginal, or rectal administration.

In some examples, the transmucosal dosage form can include an adhesive, such as acrylic adhesives, polyisobutylene adhesives, silicon adhesives, hydrogel adhesives, the like, or combinations thereof.

In some examples, the transmucosal dosage form can include a bio-erodable matrix including, but are not limited to, biodegradable constituents, such as polylactic acid, poly(lactic-co-glycolic) acid, polyglycolic acid, poly(caprolactone), hyaluronic acid, polyhydroxybutyrate, polyvinyl alcohol, polyvinylpyrrolidone, carbomers, polyacrylic acid, polyoxyethylene/polyoxypropylcne copolymers, other copolymers, albumins. casein, zein, collagen, other proteins, glucose, sucrose, maltose, trehalose, amylose, dextrose, fructose, mannose, galactose, other sugars, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, other sugar alcohols, chondroitin and/or other glycosaminoglycans, inulin, starches, acacia gum, agar, carboxymethyl cellulose, methyl cellulose, ethyl cellulose, alginates, carrageenan, cassia gums, cellulose gums, chitin, chitosan, curdlan, gelatin, dextran, fibrin, fulcelleran, gellan gum, ghatti gum, guar gum, tragacanth, karaya gum, locust bean gum, pectin, starch, tara gum, xanthan gum, and other polysaccharides, and functionalized derivatives of any of the above, copolymers thereof, the like, or mixtures thereof.

Non-limiting examples of solubilizers and/or emulsifiers can include water, ethanol, propylene glycol, ethylene glycol, glycerin, polyethylene glycol, banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof.

In some examples, the solubilizer can also include a hydrocarbon or fatty substance, such as petrolatum, microcrystalline wax, paraffin wax, mineral oil, ceresi, coconut oil, bees wax, olive oil, lanolin, peanut oil, spermaceti wax, sesame oil, almond oil, hydrogenated castor oils, cotton seed oil, soybean oil, corn oil, hydrogenated sulfated castor oils, cetyl alcohol, stearyl alcohol, oleyl alcohol, lauryl alcohol, myristyl alcohol, stearic acid, oleic acid, palmitic acid, lauraic acid, ethyl oleate, isopropyl myristicate, the like, or combinations thereof.

In some examples, the solubilizer can include a silicon, such as polydimethylsiloxanes, methicones, dimethylpropylsiloxanes, methyl phenyl polysiloxanes, steryl esters of dimethyl polysiloxanes, ethoxylated dimethicones, ethoxylated methicones, the like, or combinations thereof.

In some additional examples, the therapeutic composition can include a dispersant and/or thickening agent, such as polyacrylic acids (e.g., Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof.

The pH adjusters, tonicity agents, and preservatives in the topical, transdermal, or transmucosal therapeutic composition can generally include those pH adjusters and buffering agents, tonicity agents, and preservative agents listed above, or any other suitable pH adjusters, buffering agent, tonicity agent, or preservative for a particular formulation and/or use thereof. In some examples, the therapeutic composition can also include fumed silica, mica, talc, titanium dioxide, kaolin, aluminum glycinate, ethylenediaminetetraacetic acid, fragrances, colorants, other components as described above, the like, or combinations thereof.

In some embodiments, the patch can be a transdermal patch. When presented in the form of a transdermal patch, the transdermal drug delivery system can include various structural components. In one aspect, the transdermal patch can be a transdermal matrix patch. In one example, in the case of an adhesive matrix patch, a distal backing can be laminated to a matrix polymer laver. Such a distal backing can define the side of the matrix patch that is distal to the skin or mucosa. The backing layer can protect the matrix polymer layer and cannabinoid receptor modulator/enhancer composition and provide an impenetrable layer that prevents loss of drug to the environment. Thus, the material chosen for the backing should be compatible with the polymer layer, cannabinoid receptor modulator, and other components such as the combination of first and second enhancers, and should be minimally permeable to any components of the matrix patch. In one aspect, the backing may be opaque to protect components of the matrix patch from degradation from exposure to ultraviolet light. In another aspect, the backing can be transparent to minimize the visibility of the patch when applied. Furthermore, the backing can bind to and support the polymer layer, and be pliable enough to accommodate the movements of a subject using the matrix patch.

Suitable materials for the backing can include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. Additionally, the backing can include various foams, such as closed cell foams. Examples can include, without limitation, polyolefin foams, polyvinyl chloride foams, polyurethane foams, polyethylene foams, etc. In one aspect, the backing layer can have a thickness of about 0.0005 to about 0.1 inch.

In one aspect, the pharmaceutically acceptable carrier used in a matrix patch can be a biocompatible polymer Various general categories of biocompatible polymers can be used including, without limitation, rubbers; silicone polymers and copolymers; acrylic polymers and copolymers; and mixtures thereof. In one aspect, the biocompatible polymer can be a rubber, including natural and synthetic rubbers. One specific example of a useful rubber is a plasticized styrene-rubber block copolymer. In another aspect, the biocompatible polymer can include silicon polymers, polysiloxanes, and mixtures thereof. In yet another aspect, the biocompatible polymer can include acrylic polymers, polyacrylates, and mixtures thereof. In a further aspect, the biocompatible polymer can include vinyl acetates, ethylene-vinyl acetate copolymers, polyurethanes, plasticized polyether block amide copolymers, and mixtures thereof. In one specific aspect, the biocompatible polymer can include an acrylic copolymer adhesive such as copolymers of 2-ethylhexyacrylate and n-vinyl pyrrolidone adhesives.

Additionally, the general structure of a liquid reservoir system (LRS) type patch can be used. Such patches can comprise a fluid of desired viscosity, such as a gel or ointment, which can be formulated for confinement in a reservoir having an impermeable backing and a skin contacting permeable membrane, or a membrane adhesive laminate providing diffusional contact between the reservoir contents and the skin. The cannabinoid receptor modulator and the combination of the first and second penetration enhancers can be contained in the fluid in desired amounts. For application, a peelable release liner can be removed and the patch can be attached to the skin surface.

In one aspect, the carrier can be an ointment. An ointment is a semisolid pharmaceutical preparation including oleaginous bases, lanolins, emulsions, water-soluble bases, or combinations thereof. The ointment can contain petrolatum or zinc oxide. Oleaginous ointment bases suitable for use can include, but are not limited to, vegetable oils, animal fats, and semisolid hydrocarbons obtained from petroleum. Absorbent ointment bases can include little or no water and can include components such as, but not limited to, hydroxystearin sulfate, anhydrous lanolin, and hydrophilic petrolatum. Emulsion ointment bases can be water-in-oil (W/O) emulsions or oil-in-water (O/W) emulsions, and can include, but are not limited to, cetyl alcohol, glyceryl monostearate, lanolin, polyalkylsiloxanes, and stearic acid. Water-soluble ointment bases can be prepared from polyethylene glycols of varying molecular weight.

In another aspect, the carrier can be a cream. Creams are a type of ointment which are viscous liquids or semisolid emulsions, either oil-in-water or water-in-oil. Cream bases can be soluble in water, and can contain an oil phase, an emulsifier, an aqueous phase, or combinations thereof. In one example, the oil phase can comprise petrolatum and a fatty alcohol such as cetyl or stearyl alcohol. In another aspect, the aqueous phase can exceed the oil phase in volume, and can include a humectant. In another aspect, the emulsifier in a cream formulation can be a nonionic, anionic, cationic, or amphoteric surfactant.

In another aspect, the carrier can be a lotion. A lotion can be an ointment which can be a liquid or semi-liquid preparation in which solid particles can be present in a water or alcohol base. Lotions can be a suspension of solids or can be an oil-in-water emulsion. In another aspect, lotions can comprise suspending agents which can enhance dispersions or other compounds which can enhance contact of the cannabinoid receptor modulator with the skin. e.g., methylcellulose, sodium carboxymethylcellulose, or similar compounds.

In yet another aspect, a carrier can be a paste. Pastes can be ointments in which there are significant amounts of solids which form a semisolid formulation in which the cannabinoid receptor modulator is suspended in a suitable base. In one aspect, pastes can be formed of bases to produce fatty pastes or made from a single-phase aqueous gel. Fatty pastes can be formed of a base such as petrolatum, hydrophilic petrolatum, or the like. Pastes made from single-phase aqueous gels suitable can incorporate cellulose based polymers such as carboxymethylcellulose or the like as a base.

In another aspect, a topical gel can be prepared that includes a cannabinoid receptor modulator. A gel can be a preparation of a colloid in which a disperse phase has combined with a continuous phase to produce a viscous product. The gelling agent can form submicroscopic crystalline particle groups that retain the solvent in the interstices. Gels can be semisolid, suspension-type systems. Single-phase gels can contain organic macromolecules distributed substantially uniformly throughout a carrier liquid, which can be aqueous or non-aqueous and can contain an alcohol or oil.

In some embodiments, the combination of first (e.g., cetyl/sorbitan palmitate) and second penetration enhancers (e.g., dimethyl ether isosorbide, a terpene, or a combination thereof) can facilitate an extended therapeutic effect for a topical composition having a cannabinoid receptor modulator relative to a topical composition without the combination of first (e.g., cetyl/sorbitan palmitate) and second penetration enhancers (e.g., dimethyl ether isosorbide, a terpene, or a combination thereof). In one aspect, the extended therapeutic effect can have a duration of from about 1 second to about one week. In one example, the extended therapeutic effect can have a duration of from about 30 minutes to about 5 days. In another example, the extended therapeutic effect can have a duration of from about 4 hours to about 3 days.

In another aspect, the combination of first (e.g., cetyl/sorbitan palmitate) and second penetration enhancers (e.g., dimethyl ether isosorbide, a terpene, or a combination thereof) can facilitate a rapid therapeutic effect that commences within a selected time. In one aspect, the therapeutic effect is rapid when the therapeutic effect commences within a selected time that can be less than one or more of: 1 second, 5 seconds, 30 seconds, 2 minutes, 5 minutes, 15 minutes, 30 minutes, 1 hour, 4 hours, 8 hours, the like, or a combination thereof.

In some embodiments, the combination of first (e.g., cetyl/sorbitan palmitate) and second penetration enhancers (e.g., dimethyl ether isosorbide, a terpene, or a combination thereof) can facilitate an extended therapeutic effect, a rapid therapeutic effect, or a combination thereof for the topical composition relative to a therapeutic effect duration or a therapeutic effect onset time for a topical composition without the combination of first (e.g., cetyl/sorbitan palmitate) and second penetration enhancers (e.g., dimethyl ether isosorbide, a terpene, or a combination thereof).

In some embodiments, the combination of first (e.g., cetyl/sorbitan palmitate) and second penetration enhancers (e.g., dimethyl ether isosorbide, a terpene, or a combination thereof) can facilitate a greater or substantially equivalent therapeutic effect duration or reduced or substantially equivalent therapeutic effect onset time when compared for a reduced dose of the cannabinoid receptor modulator, when compared to a therapeutic effect duration or a therapeutic effect onset time for an increased dose of the cannabinoid receptor modulator for a topical composition without the combination of first (e.g., cetyl/sorbitan palmitate) and second penetration enhancers (e.g., dimethyl ether isosorbide, a terpene, or a combination thereof). In one aspect, the reduced dose can be less than the increased dose by one or more of: 5%, 10%, 20%, 35%, 50%, 1 mg, 5 mg, 10 mg, 25 mg, or a combination thereof. For example, a reduced dose of 25 mg can be considered to be 50% less than an increased dose of 50 mg.

In one aspect, the therapeutic effect duration or onset time for a reduced dose of the cannabinoid receptor modulator can be considered to be substantially equivalent when the therapeutic effect duration difference or onset time difference is less than 5%, or 10%, or 20%, 35%, or 50% relative to the therapeutic effect duration for the increased dose of the cannabinoid receptor modulator. For example, a therapeutic effect duration or onset time of 95 minutes can be considered to be within a 5% difference of a therapeutic effect duration or onset time of 100 minutes. In another aspect, the therapeutic effect duration or onset time for a reduced dose of the cannabinoid receptor modulator can be considered to be substantially equivalent when the therapeutic effect duration differs by less than 1 second, 5 seconds, 30 seconds, 2 minutes, 5 minutes, 15 minutes, 30 minutes, 1 hour, 4 hours, 8 hours relative to the therapeutic effect duration for the increased dose of the cannabinoid receptor modulator. For example, a therapeutic effect duration or onset time of 5 minutes can be considered to be within 5 minutes of a therapeutic effect duration or onset time of 1 minute.

In another aspect, the combination of first (e.g., cetyl/sorbitan palmitate) and second penetration enhancers (e.g., dimethyl ether isosorbide, a terpene, or a combination thereof) can facilitate a substantially equivalent therapeutic effect duration or therapeutic effect onset time for a reduced daily dose of the cannabinoid receptor modulator relative to a therapeutic effect duration or a therapeutic effect onset time for an increased daily dose of the cannabinoid receptor modulator for a topical composition without the combination, wherein the reduced daily dose is less than the increased daily dose by one or more of: 5%, 10%, 20%, 35%, 50%, 1 mg, 5 mg, 10 mg, 25 mg, or a combination thereof.

In another aspect, the combination of first (e.g., cetyl/sorbitan palmitate) and second penetration enhancers (e.g., dimethyl ether isosorbide, a terpene, or a combination thereof) can facilitate a substantially equivalent therapeutic effect duration or therapeutic effect onset time for a reduced dose frequency of the cannabinoid receptor modulator relative to a therapeutic effect duration or a therapeutic effect onset time for an increased dose frequency of the cannabinoid receptor modulator for a topical composition without the combination, wherein the reduced dose frequency is less than the increased dose frequency by one or more of: once per week, twice per week, three time per week, once per day, twice per day, three times per day, five times per day, or a combination thereof.

### Methods

In some embodiments there are provided methods of treatment utilizing topical cannabinoid administration to a subject. In one embodiment, a method of treating a condition in a subject that is responsive to treatment with a cannabinoid receptor modulator can comprise administering a therapeutically effective amount of the topical composition as disclosed herein to the subject.

In one aspect, a lipophilic terpene (e.g., terpinolene, D-limonene and β-caryophyllene) when combined with a penetration enhancer (e.g., cetyl/sorbitan palmitate) can disrupt lipid packing in a stratum comeum of a subject. The disruption of the lipid packing can facilitate a greater combined therapeutic effect than provided by a sum of individual therapeutic effects of the individual components of the penetration enhancer and the lipophilic terpene.

In one aspect, a lipophilic terpene (e.g., terpinolene, D-limonene and β-caryophyllene) when combined with an amphiphilic penetration enhancer (e.g., dimethyl isosorbide, hydrotropes thereof, like diethylene glycol monoethyl ether, ethylene glycol monododecyl ether, adipic acid diisopropyl ester, diethyl sebacate) can disrupt lipid packing in a stratum corneum of a subject. The disruption of the lipid packing can facilitate a greater combined therapeutic effect than provided by a sum of individual therapeutic effects of the individual components of the amphiphilic penetration enhancer and the lipophilic terpene.

In another aspect, an amphiphilic penetration enhancer (e.g., dimethyl isosorbide, hydrotropes thereof, like diethylene glycol monoethyl ether, ethylene glycol monododecyl ether, adipic acid diisopropyl ester, diethyl sebacate) when combined with a penetration enhancer (e.g., cetyl/sorbitan palmitate) can disrupt lipid packing in a stratum corneum of a subject. The disruption of the lipid packing can facilitate a greater combined therapeutic effect than provided by a sum of individual therapeutic effects of the individual components of the penetration enhancer and the amphiphilic penetration enhancer.

In another aspect, a lipophilic terpene (e.g., terpinolene, D-limonene and β-caryophyllene) when combined with an amphiphilic penetration enhancer (e.g., dimethyl isosorbide, hydrotropes thereof, like diethylene glycol monoethyl ether, ethylene glycol monododecyl ether, adipic acid diisopropyl ester, diethyl sebacate) and a penetration enhancer (e.g., cetyl/sorbitan palmitate) can disrupt lipid packing in a stratum corneum of a subject. The disruption of the lipid packing can facilitate a greater combined therapeutic effect than provided by a sum of individual therapeutic effects of the individual components of the lipophilic terpene, amphiphilic penetration enhancer and penetration enhancer (e.g., cetyl/sorbitan palmitate).

In one aspect, the condition can be pain. In one aspect, the pain can be one or more of acute pain, chronic pain, neuropathic pain, nociceptive pain, radicular pain, the like, or combinations thereof.

In one example, acute pain can have a duration of 5 seconds to about three months. In another example, acute pain can be caused by or associated with a soft-tissue injury, which includes bruising, or fleeting illness.

In another example, chronic pain can have a duration persisting from several months to the lifetime of a subject and can be exacerbated by persistent inflammation. In one example, chronic pain can include, but is not limited to: (i) arthritic pain caused by inflammation of joints due to infectious, metabolic and constitutional causes, including wear-and-tear of joint and vertebral cartilages, (ii) backaches caused by spine conditions (e.g., degenerative disc disease, spinal stenosis, acute injuries, and spondylolisthesis), (iii) fibromyalgia, and (iv) tendinitis.

In another example, neuropathic pain can be caused by damage to the nerves or other components of the nervous system. Neuropathic pain can encompass shooting, stabbing, or burning pain which can be a type of pain that can persist for multiple years.

In another example, nociceptive pain can be acute pain, chronic pain, or a combination thereof caused by damage to body tissue, (e.g., external injury) and can be characterized as achy or throbbing pain caused.

In yet another example, the radicular pain can be pain caused by spinal nerve compression and inflammation. Radicular pain can emanate from the back, hip, and legs via the spine and spinal nerve root and can be characterized by tingling, numbness, and muscle weakness. In one example, radicular pain can include sciatica on one side of the body of a subject. Radicular pain can be caused by a herniated disk, spinal bone spur, or spinal stenosis (e.g., caused by a tumor) that can compress a nerve. Radicular pain can cause damaging inflammation, pain, and numbness in the affected leg. Sciatica, which can be caused by compression of a spinal nerve root in the lower back, can be caused by degeneration of an intervertebral disk.

In one aspect, topical cannabinoid administration to a subject can treat pain that is one or more of joint pain, muscle pain, arthritis, fibromyalgia, or a combination thereof. In one aspect, the pain can be induced by one or more of peripheral nerve damage, inflammation, cancer, cancer treatment, burns, surgery, the like, or a combination thereof. In another aspect, topical cannabinoid administration to a subject can treat edema or inflammation.

In one aspect, administration of a suitable cannabinoid receptor modulator, terpene, analgesic (e.g., CBD, β-caryophyllene, myrcene, palmitoylethanolamide (PEA), histamine dihydrochloride, menthol) can enhance the alleviation of pain symptoms, inflammation symptoms, or a combination thereof.

In one aspect, topical administration of the topical composition disclosed herein (e.g., including CBD) can enhance the treatment of pain. In one example, CBD can weakly bind to cannabinoid receptor type 1 (CB1) to facilitate positive allosteric modulation of serotonin to provide an alleviation of pain. In another aspect, CBD can bind to G-coupled receptors, specifically, the μ- and δ-opioid receptors, and, unlike fentanyl, morphine, heroin, methadone and other opioids, can activate analgesia associated with G-coupled receptors in a non-addictive method. In yet another aspect, CBD can inhibit fatty acid amide hydrolase to provide increased levels of anandamide.

In one aspect, topical administration of the topical composition disclosed herein (e.g., including β-Caryophyllene) can enhance the treatment of pain and inflammation. In one aspect, β-Caryophyllene can mitigate pain by binding to cannabinoid receptor type 2 (CB2) to reduce inflammation. In another aspect. β-Caryophyllene can antagonistically interact with peroxisome proliferator-activated receptors to reduce pain.

In one aspect, topical administration of the topical composition disclosed herein (e.g., including myrcene) can enhance the treatment of acute pain. In another aspect, myrcene can potently inactivate the TRPV1 receptor to facilitate a reduction in acute pain with a duration of analgesia that is greater than the reduction in acute pain caused by morphine. In one aspect, antagonism of a TRPV1 receptor can provide acute analgesia.

In one aspect, topical administration of the topical composition disclosed herein (e.g., including PEA) can enhance the treatment of pain and inflammation. PEA can positively affect the pharmacodynamic impact of anandamide (ANA) by inhibiting FAAH expression. ANA can originate via the non-oxidative metabolism of arachidonic acid through multiple synthetic pathways. ANA can strongly bind to the CB1 receptor to prevent pain signaling. Fatty acid amide hydrolase (FAAH) is responsible for the hydrolysis, or degradation, of ANA3; therefore, degradation of FAAH can enhance the alleviation of pain. In one aspect, hindering ANA destruction can increase ANA concentration and facilitate increased binding of ANA to the CB1 receptor to provide enhanced analgesia. In another aspect, PEA, as an agonist of the peroxisome proliferator-activated receptor-α (PPAR-α) can suppress pain induced by chemical injury, nerve damage, or inflammation.

In another aspect, topical administration of the topical composition disclosed herein (e.g., including histamine dihydrochloride) can enhance the treatment of pain, joint pain, muscle pain, or a combination thereof. Histamine dihydrochloride, when administered to the skin, is a vasodilator that can expand the diameter of blood vessels and increase blood circulation. This vasodilation can mimic the natural response to pain. Consequently, histamine dihydrochloride can reduce pain in a joint or muscle. Histamine dihydrochloride, acting on distinct histamine H1, H2, H3, and H4 receptors, can generally alleviate pain.

In another aspect, topical administration of the topical composition disclosed herein (e.g., including menthol) can enhance the treatment of pain, acute pain, inflammatory pain, or a combination thereof. Menthol can be a selective activator of transient receptor potential melastatin-8 (TRPM8) channels, which mediate menthol-induced analgesia for acute and inflammatory pain. The cooling sensation is achieved by activating TRPM8 in the cold-sensitive peripheral sensory neurons of the skin while simultaneously triggering central pathways associated with analgesia. In another aspect, the analgesic activity of menthol an be further enhanced through selective activation of κ-opioid receptors, which can directly inhibit neurons to constrain pain transmission.

In another aspect, topical cannabinoid administration to a subject can treat a condition including one or more of a neurological condition, a psychological condition, a digestive condition, a heart condition, a kidney condition, a bladder condition, a liver condition, a skin condition, an immune disorder, an endocrine disorder, a breathing disorder, the like, or combinations thereof.

In one aspect, the condition can comprise one or more of Alzheimer's disease, dementia, amyotrophic lateral sclerosis (ALS), epilepsy, seizures, multiple sclerosis, muscle spasms, Parkinson's disease, stroke, brain injury, headaches, migraines, the like, or combinations thereof.

In another aspect, the condition can comprise one or more of a sleep disorder, anxiety, depression, ADD. ADHD, autism spectrum disorder, schizophrenia, psychosis, bipolar disorder, addiction, alcoholism, post-traumatic stress disorder, stress, the like, or combinations thereof.

In one aspect, the condition can comprise one or more of HIV, Crohn's disease, glaucoma, cancer, diabetes, osteoporosis, nausea, obesity, antibiotic resistance, prion disease, endometriosis, chronic fatigue syndrome, the like, or combinations thereof.

In some embodiments, the topical composition can be administered according to a suitable dosage regimen. In one aspect, the dosage form can have from about 25 ul to about 10 ml of the topical composition. In another example, the dosage form can have from about 100 ul to about 0.75 ml of the topical composition. In another example, a daily dose can be from about 25 ul ml to about 10 ml of the topical composition. In another example, a daily dose can be from about 1 ml to about 5 ml of the topical composition.

In one more example, the dosage interval can vary based on the number of previous doses administered in a selected time window and a duration of administration. In one example, the therapeutically effective amount of the topical composition can be administered to a subject at least two different dosage intervals. The first dosage interval (e.g., the time between the first dose administered and the second dose administered in a day) can be a selected interval between 30 minute and 12 hours and the second dosage interval (e.g., the time between the second dose administered and the third dose administered in a day) can be a selected interval between 30 minutes and 12 hours. In some examples, the first dosage interval can be lower than a second dosage interval. For example, a subject who receives a first dose at 9 am, a second dose at 12 μm, and a third dose at 10 pm would have a first dosage interval of about 3 hours and a second dosage interval of about 10 hours. In this example, the dosage interval can lengthen over the course of a day. In other example, the dosage interval can lengthen over the course of a week, 2 weeks, 4 weeks, 8 weeks, 3 months, 6 months, 1 years, the like, and combinations thereof.

In another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with pain. In one aspect, topical cannabinoid administration to a subject can facilitate a reduction in symptoms of at least 5%, 10%, 15%, 20%, 30%, 35%, or 50% within a selected amount of time (e.g., 5 seconds, 1 minute, 5 minutes, 15 minutes, 30 minutes, 60 minutes, 4 hours, 8 hours, 1 day, 3 days, 1 week) after administration. In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In one aspect, the symptoms of the condition can be reduced by at least 5%, 10%, 15%, 20%, 30%, 35%, or 50%, 80%, 90% since commencement of the treatment. In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

### EXAMPLES

The following example is provided to promote a clearer understanding of certain embodiments of the present disclosure, and are in no way meant as a limitation thereon.

### Example 1—Manufacture of a Skin Cream

Although dimethyl isosorbide is herein used, similar results can be attained using other amphiphilic penetration enhancers like diethylene glycol monoethyl ether, ethylene glycol monododecyl ether, adipic acid diisopropyl ester and diethyl sebacate, other ethers, and other esters. The terpenes used to complement permeation are biasbolol, β-caryophyllene, D-limonene, linalool, niaouli oil, myrcene and terpinolene. However, there are more than 30,000 unsaturated hydrocarbons made by plants classified as terpenes, encompassing monoterpenes (C₁₀) and sesquiterpenes (C₁₅), diterpenes (C₂₀) and more. Many of these terpenes possess attributes that can be useful for enhancing the movement of active agents (e.g., cannabinoid receptor modulators) into or through the skin. Table 1-A presents an example of the preparation of a topical composition as disclosed herein. Table 1-B presents another example including the concentration ranges for each ingredient. Tables 1-C, 1-D, and 1-E present additional examples including the concentration ranges for each ingredient.

TABLE 1-A

| | | VOLUME |
|---|---|---|
| PHASE | INGREDIENT | % Weight |
| A | Octanoic/Decanoic Acid Triglyceride (Caprylic/ | 10.0 |
| | Capric Triglyceride) | |
| A | Ceterayl/Sorbitan Olivate | 6.5 |
| A | Cetyl/Sorbitan Palmitate | 2.5 |
| A | Polyacrylate Crosspolymer-6 (Copolymer of | 0.4 |
| | Ammonium Acryloyldimethyltaurate, | |
| | Dimethylacrylamide, Lauryl Methacrylate and | |
| | Laureth-4 Methacrylate, Crosslinked with | |
| | Trimethylolpropane Triacrylate) | |
| B | Distilled Water | q.s. |
| B | Sorbitan Gum Powder (Scleroglucan) | 0.4 |
| C | Cannabidiol (CBD) | 2.0 |
| C | Dimethyl Isosorbide (Dimethyl Ether Isosorbide) | 5.0 |
| D | Palmitoylethanolamide (N-(2- | 2.0 |
| | hydroxyethyl)hexadecanamide) | |
| D | Biopolysan 220B (Benzyl Alcohol, Diglycerine and | 2.5 |
| | Polyglyceryl-2-Laurate) | |
| D | Helianthus Annus Seed Oil, Laurel Laurate, Cetyl | 2.0 |
| | Palmitate (DF Sunflower) | |
| D | Bisabolol | 0.5 |
| D | β-Caryophyllene | 2.2 |
| D | D-Limonene | 0.5 |
| D | Linalool | 0.5 |
| D | Melaleuca Quinquenervia Oil (Niaouli Oil) | 0.5 |
| D | Myrcene | 0.5 |
| D | Terpinolene | 0.5 |

Preparation: 1. Mix Phase A ingredients and heat to 75-80° C. Disperse using a Silverson dispersing unit with square hole screen. 2. Mix Phase B ingredients and heat to 70-80° C. Disperse using a Silverson dispersing unit with square hole screen. 3. Add Phase B to Phase A under agitation using the Silverson dispersing unit with an Emulsor screen. 4. Cool the combined mixture to less than 35° C. and gently stir to fully mix. 5. Prepare Phase C ingredients by allowing CBD to dissolve in dimethyl isosorbide while stirring with a spin bar or blade mixer. 6. Combine Phase D ingredients, other than Palmitoylethanolamide. Gently stir with a spin bar until completely mixed. Add Palmitoylethanolamide last and mix until it has been fully dissolved. 7. Add Phase D to Phase C while stirring with a spin bar or blade mixer. 8. Combine Phase A/B and Phase C/D. Fully mix while stirring with a spin bar or blade mixer.

TABLE 1-B

| | | VOLUME % | |
|---|---|---|---|
| PHASE | INGREDIENT | Weight | RANGE |
| A | Octanoic/Decanoic Acid Triglyceride | 10.0 | 7.0-14.0 |
| | (Caprylic/Capric Triglyceride) | | |
| A | Ceterayl/Sorbitan Olivate | 6.5 | 3.5-8.0 |
| A | Cetyl/Sorbitan Palmitate | 2.5 | 1.0-5.5 |
| A | Polyacrylate Crosspolymer-6 | 0.4 | 0.1-0.8 |
| | (Copolymer of Ammonium | | |
| | Acryloyldimethyltaurate, | | |
| | Dimethylacrylamide, Lauryl | | |
| | Methacrylate and Laureth-4 | | |
| | Methacrylate, Crosslinked with | | |
| | Trimethylolpropane Triacrylate) | | |
| B | Distilled Water | q.s. | — |
| B | Sorbitan Gum Powder (Scleroglucan) | 0.4 | — |
| C | Cannabidiol (CBD) | 2.0 | 0.1-15.0 |
| C | Dimethyl Isosorbide (Dimethyl | 5.0 | 1.0-10.0 |
| | Ether Isosorbide) | | |
| D | Palmitoylethanolamide (N-(2- | 2.0 | 0.5-10.0 |
| | hydroxyethyl)hexadecanamide) | | |
| D | Biopolysan 220B (Benzyl Alcohol, | 2.5 | 1.0-5.5 |
| | Diglycerine and Polyglyceryl-2- | | |
| | Laurate) | | |
| D | Helianthus Annus Seed Oil, | 2.0 | 1.0-3.5 |
| | Laurel Laurate, Cetyl Palmitate | | |
| | (DF Sunflower) | | |
| D | Bisabolol | 0.5 | 0.1-3.0 |
| D | β-Caryophyllene | 2.2 | 1.0-6.0 |
| D | D-Limonene | 0.5 | 0.1-2.0 |
| D | Linalool | 0.5 | 0.1-25 |
| D | Melaleuca Quinquenervia Oil | 0.5 | 0.2-3.0 |
| | (Niaouli Oil) | | |
| D | Myrcene | 0.5 | 0.2-5.0 |
| D | Terpinolene | 0.5 | 0.1-1.0 |
| D | Histamine Dihydrochloride | 0.05 | — |
| D | Menthol | 5.0% | 2.0-10.0 |

TABLE 1-C

| | | VOLUME % | |
|---|---|---|---|
| PHASE | INGREDIENT | Weight | RANGE |
| A | Octanoic/Decanoic Acid Triglyceride | 10.0 | 7.0-14.0 |
| | (Caprylic/Capric Triglyceride) | | |
| A | Ceterayl/Sorbitan Olivate | 6.5 | 3.5-8.0 |
| A | Cetyl/Sorbitan Palmitate | 2.5 | 1.0-5.5 |
| A | Polyacrylate Crosspolymer-6 | 0.4 | 0.1-0.8 |
| | (Copolymer of Ammonium | | |
| | Acryloyldimethyltaurate, | | |
| | Dimethylacrylamide, Lauryl | | |
| | Methacrylate and Laureth-4 | | |
| | Methacrylate, Crosslinked with | | |
| | Trimethylolpropane Triacrylate) | | |
| B | Distilled Water | q.s. | — |
| B | Sorbitan Gum Powder (Scleroglucan) | 0.4 | — |
| C | Cannabidiol (CBD) | 2.0 | 0.1-15.0 |
| C | Dimethyl Isosorbide (Dimethyl | 5.0 | 1.0-10.0 |
| | Ether Isosorbide) | | |
| D | Palmitoylethanolamide (N-(2- | 2.0 | 0.5-10.0 |
| | hydroxyethyl)hexadecanamide) | | |
| D | Biopolysan 220B (Benzyl Alcohol, | 2.5 | 1.0-5.5 |
| | Diglycerine and Polyglyceryl-2- | | |
| | Laurate) | | |

TABLE 1-D

| | | VOLUME % | |
|---|---|---|---|
| PHASE | INGREDIENT | Weight | RANGE |
| A | Octanoic/Decanoic Acid Triglyceride | 10.0 | 7.0-14.0 |
| | (Caprylic/Capric Triglyceride) | | |
| A | Ceterayl/Sorbitan Olivate | 6.5 | 3.5-8.0 |
| A | Polyacrylate Crosspolymer-6 | 0.4 | 0.1-0.8 |
| | (Copolymer of Ammonium | | |
| | Acryloyldimethyltaurate, | | |
| | Dimethylacrylamide, Lauryl | | |
| | Methacrylate and Laureth-4 | | |
| | Methacrylate, Crosslinked with | | |
| | Trimethylolpropane Triacrylate) | | |
| B | Distilled Water | q.s. | — |
| B | Sorbitan Gum Powder (Scleroglucan) | 0.4 | — |
| C | Cannabidiol (CBD) | 2.0 | 0.1-15.0 |
| C | Dimethyl Isosorbide (Dimethyl | 5.0 | 1.0-10.0 |
| | Ether Isosorbide) | | |
| D | Palmitoylethanolamide (N-(2- | 2.0 | 0.5-10.0 |
| | hydroxyethyl)hexadecanamide) | | |
| D | Biopolysan 220B (Benzyl Alcohol, | 2.5 | 1.0-5.5 |
| | Diglycerine and Polyglyceryl-2- | | |
| | Laurate) | | |
| D | Bisabolol | 0.5 | 0.1-3.0 |
| D | β-Caryophyllene | 2.2 | 1.0-6.0 |
| D | D-Limonene | 0.5 | 0.1-2.0 |
| D | Linalool | 0.5 | 0.1-25 |
| D | Melaleuca Quinquenervia Oil | 0.5 | 0.2-3.0 |
| | (Niaouli Oil) | | |
| D | Myrcene | 0.5 | 0.2-5.0 |
| D | Terpinolene | 0.5 | 0.1-1.0 |
| D | Histamine Dihydrochloride | 0.05 | — |
| D | Menthol | 5.0% | 2.0-10.0 |

TABLE 1-E

| | | VOLUME % | |
|---|---|---|---|
| PHASE | INGREDIENT | Weight | RANGE |
| A | Octanoic/Decanoic Acid Triglyceride | 10.0 | 7.0-14.0 |
| | (Caprylic/Capric Triglyceride) | | |
| A | Ceterayl/Sorbitan Olivate | 6.5 | 3.5-8.0 |
| A | Cetyl/Sorbitan Palmitate | 2.5 | 1.0-5.5 |
| A | Polyacrylate Crosspolymer-6 | 0.4 | 0.1-0.8 |
| | (Copolymer of Ammonium | | |
| | Acryloyldimethyltaurate, | | |
| | Dimethylacrylamide, Lauryl | | |
| | Methacrylate and Laureth-4 | | |
| | Methacrylate, Crosslinked with | | |
| | Trimethylolpropane Triacrylate) | | |
| B | Distilled Water | q.s. | — |
| B | Sorbitan Gum Powder (Scleroglucan) | 0.4 | — |
| C | Cannabidiol (CBD) | 2.0 | 0.1-15.0 |
| D | Palmitoylethanolamide (N-(2- | 2.0 | 0.5-10.0 |
| | hydroxyethyl)hexadecanamide) | | |
| D | Biopolysan 220B (Benzyl Alcohol, | 2.5 | 1.0-5.5 |
| | Diglycerine and Polyglyceryl-2- | | |
| | Laurate) | | |
| D | Helianthus Annus Seed Oil, Laurel | 2.0 | 1.0-3.5 |
| | Laurate, Cetyl Palmitate | | |
| | (DF Sunflower) | | |
| D | Bisabolol | 0.5 | 0.1-3.0 |
| D | β-Caryophyllene | 2.2 | 1.0-6.0 |
| D | D-Limonene | 0.5 | 0.1-2.0 |
| D | Linalool | 0.5 | 0.1-25 |
| D | Melaleuca Quinquenervia Oil | 0.5 | 0.2-3.0 |
| | (Niaouli Oil) | | |
| D | Myrcene | 0.5 | 0.2-5.0 |
| D | Terpinolene | 0.5 | 0.1-1.0 |
| D | Histamine Dihydrochloride | 0.05 | — |
| D | Menthol | 5.0% | 2.0-10.0 |

### Example 2—Topical Compositions

Table 2-A, 2-B, and 2-C provide examples of topical compositions.

TABLE 2-A

| | | VOLUME % | |
|---|---|---|---|
| PHASE | INGREDIENT | Weight | RANGE |
| A | Triglyceride | 10.0 | 7.0-14.0 |
| A | Hexadeconic acid, Hexadeconic acid | 2.5 | 1.0-5.5 |
| | salt, Hexadeconic acid ester | | |
| A | Thickening Agent | 0.4 | 0.1-0.8 |
| B | Distilled Water | q.s. | — |
| B | Emulsifier | 0.4 | — |
| C | Cannabinoid receptor modulator | 2.0 | 0.1-15.0 |
| C | Amphiphilic penetration enhancer | 5.0 | 1.0-10.0 |
| D | Penetration Enhancer | 2.0 | 0.5-10.0 |

TABLE 2-B

| | | VOLUME % | |
|---|---|---|---|
| PHASE | INGREDIENT | Weight | RANGE |
| A | Triglyceride | 10.0 | 7.0-14.0 |
| A | Thickening Agent | 0.4 | 0.1-0.8 |
| B | Distilled Water | q.s. | — |
| B | Emulsifier | 0.4 | — |
| C | Cannabinoid receptor modulator | 2.0 | 0.1-15.0 |
| C | Amphiphilic penetration enhancer | 5.0 | 1.0-10.0 |
| D | Penetration Enhancer | 2.0 | 0.5-10.0 |
| D | Terpene | 0.5 | 0.1-50 |

TABLE 2-C

| | | VOLUME % | |
|---|---|---|---|
| PHASE | INGREDIENT | Weight | RANGE |
| A | Triglyeride | 10.0 | 7.0-14.0 |
| A | Hexadeconic acid, Hexadeconic acid | 2.5 | 1.0-5.5 |
| | salt, Hexadeconic acid ester | | |
| A | Thickening Agent | 0.4 | 0.1-0.8 |
| B | Distilled Water | q.s. | — |
| B | Emulsifier | 0.4 | — |
| C | Cannabinoid receptor modulator | 2.0 | 0.1-15.0 |
| D | Penetration Enhancer | 2.0 | 0.5-10.0 |
| D | Terpene | 0.5 | 0.1-50 |

### Example 3—Treatment of Pain

An open label study on human subjects was done to assess the utility of the formulation in Table 1-A as a therapeutic agent for treating pain and associated inflammation. Subjects were treated once on each of 3 consecutive days, and this treatment was quantified as follows: (i) an initial assessment prior to application of the formulation, typically soon after awakening; (ii) a pain assessment 30 minutes post administration; and (iii) a pain assessment done at 3 hours after application of the formulation.

Acute pain from trauma, chronic pain stemming from tissue degeneration and osteoarthritis, and peripheral neuropathy were examined. The acute pain herein addressed came from muscle contusion, or bruising. Osteoarthritis, or degenerative joint disease, is not only a frequent source of chronic pain but also the most common joint disorder in the United States. About 1 in 4 American adults suffer from some form of osteoarthritis. It is a major reason for lumbago, or lower back pain, and is typically associated with progressive vertebral disk degeneration. Around 40% of people more than 30 years old are dealing with chronic back pain. Peripheral neuropathy is a condition involving nerve damage in the lower legs, feet and hands that can cause severe pain in the shins, feet and hands. At least 20 million Americans have some form of peripheral neuropathy, and this condition will impact approximately 50% of adult diabetics during their lifetimes.

### Methodology

A single daily application of approximately 0.75-mL of the formulation in Table 1-A was applied to the area affected by pain and massaged until thoroughly absorbed into skin. After application of the formulation, subjects were instructed to pursue their usual daily routine other than to quantitatively assess their pain levels using the Face, Legs, Activity, Cry, and Consolability (FLACC) scale. FLACC is a numerical scale for pain ranging from 0 to 10, in which 0 means no pain, 1-3 mild pain, 4-7 moderate pain, and 8 or above severe pain. Such enumeration allows for dependable intra-individual assessments of pain severity across time Pain measures were the FLACC scores during the first 3 hours. From Day 1 through Day 3, a FLACC score were measured: (a) immediately before applying the formulation, (b) 30 minutes post application, and (c) 3 hours post application.

### Results

FLACC scale results are summarized in Table 3.

TABLE 3

| | | Day 1 | | | Day 2 | | | Day 3 | | |
|---|---|---|---|---|---|---|---|---|---|---|
| | | | 30 | 3 | | 30 | 3 | | 30 | 3 |
| Subject | Condition | T₀ | Min. | Hours | T₀ | Min. | Hours | T₀ | Min. | Hours |
| 1 | Acute Pain Bruised | 7 | 5 | 3 | 5 | 2 | 3 | 4 | 3 | 2 |
| | Left Thigh | | | | | | | | | |
| 2 | Osteoarthritis | 7 | 4 | 1 | 5 | 3 | 2 | 6 | 4 | 2 |
| | Both Wrists | | | | | | | | | |
| 3 | Lumbar Osteoarthritis | 8 | 4 | 3 | 6 | 4 | 2 | 7 | 5 | 3 |
| 4 | Lumbar Osteoarthritis | 7 | 5 | 3 | 5 | 4 | 2 | 6 | 4 | 3 |
| 5 | Peripheral | 8 | 4 | 2 | 3 | 2 | 2 | 2 | 1 | 2 |
| | Neuropathy Shin | | | | | | | | | |
| 6 | Acute Pain Bruised | 6 | 3 | 3 | 7 | 5 | 4 | 6 | 3 | 2 |
| | Back Right Scapula | | | | | | | | | |
| 7 | Lumbar Osteoarthritis | 5 | 3 | 3 | 6 | 2 | 3 | 5 | 4 | 3 |
| 8 | Peripheral Neuropathy | 8 | 5 | 5 | 7 | 5 | 3 | 6 | 4 | 3 |
| | Right Foot | | | | | | | | | |

Table 3 shows Face, Legs, Activity, Cry, and Consolability (FLACC) Scores Across 3 Days of administration of the formulation. To refers to pain assessments made immediately prior to administration of the formulation on a given day.

Average FLACC scores prior to using formulation (T)) were less, 6.1, on Day 2 and 5.1 at Day 3. For 30 minutes post administration, average FLACC scores were on 4.1 on Day 1, 3.4 on Day 2, and at 3.5 on Day 3. As for FLACC values for 3 hours after administration of the formulation, these were 2.9 on Day 1, 2.6 on Day 2, and 2.5 on Day 3.

A Mann-Whitney test, the non-parametric equivalent of the parametric independent samples t-test, was used to test these results for significance. At 3 hours after administering the formulation, a significant decrease in pain was experienced on all 3 days (Day 1: Mann-Whitney U=0.5, n₁=n₂=8, p<0.01 two tailed; Day 2: Mann-Whitney U=10, n₁=n₂=8, p<0.05 two tailed: Day 3: Mann-Whitney U=6, n₁=n₂=8, p<0.01 two tailed).

At 30 minutes after administering the formulation, a significant decrease in pain was experienced on all 3 days (Day 1: Mann-Whitney U=1.5, n₁=n₂=8, p<0.01 two tailed; Day 2: Mann-Whitney U=7.5, n₁=n₂=8, p<0.05 two tailed: Day 3: Mann-Whitney U=10.5, n₁=n₂=8, p<0.05 two tailed).

Day-to-day comparisons were made of the results for 3 hours post administration. The outcome for Day 2 versus Day 1 was not significant (Mann-Whitney U=26.5, n₁=n₂=8, not significant at p<0.05 two tailed), nor were those for Day 3 versus either Day 1 (Mann-Whitney U=24, n₁=n₂=8, not significant at p<0.05 two tailed) or Day 2 (Mann-Whitney U=30, n₁=n₂=8, not significant at p<0.05 two tailed).

Pre-administration pain levels on Day 3 were significantly less than pre-administration levels on Day 1 (Mann-Whitney U=10, n₁=n₂=8, p<0.05 two tailed), as were those for Day 2 versus Day 1 (Mann-Whitney U=11.5, n₁=n₂=8, p<0.05 two tailed).

### Summary:

The following statistics-based statements can thus be made: (1) Both acute and chronic pain are successfully mitigated by applications of the formulation. Pain is significantly lessened over a 3-hour time span in any given day. (2) Analgesia from the formulation has a rapid onset of action. Within 30 minutes after its application, significant levels of pain relief are realized. (3) There is a cumulative analgesic effect achieved from using the formulation in Table 1-A as evidenced by decreasing FLACC scores decreasing over the 3 days of the study. The formulation on a given day sets the stage for a lesser level of pain at the beginning of the next day. This carryover effect manifests even before the formulation is applied at the beginning of the day.

### Example Embodiments

In one embodiment, a topical composition can comprise: a therapeutically effective amount of at least one cannabinoid receptor modulator; an amount of a combination of a first and second penetration enhancer at synergistic ratios that facilitate a greater combined therapeutic effect than provided by a sum of individual therapeutic effects of the individual components of the combination; and a pharmaceutically acceptable carrier suitable for topical administration.

In one aspect, the at least one cannabinoid receptor modulator and the combination of first and second penetration enhancers can be present in a ratio of from about 20:1 to about 1:500. In another aspect, the first and second penetration enhancers can be present in a ratio of from about 10:1 to about 1:50.

In another aspect, the second penetration enhancer can comprise at least one terpene. In one example, the at least one cannabinoid receptor modulator and the at least one terpene can be present in a ratio of from about 10:1 to about 1:100. In another example, the first penetration enhancer and the at least one terpene can be present in a ratio of from about 5:1 to about 1:50.

In another aspect, the at least one terpene can be a cannabis extract. In another aspect, the at least one terpene can be a lipophilic terpene.

In another aspect, the at least one terpene can have a c Log P of greater than 0. In one example, the at least one terpene can be a member selected from the group consisting of: (+)-aromadendrene, (+)-cedrol, (+)-cedryl acetate, (+)-dihydrocarveol, (+)-dihydrocarvone, (+)-longifolene, (+)-β-cedrene, (−)-carveol, (−)-caryophyllene oxide, (−)-dihydrocarveol, (−)-epiglobulol, (−)-guaiol, (−)-isolongifolol, (−)-isopulegol, (−)-trans-caryophyllene, (−)-α-cedrene, (−)-α-santonin, (−)-α-thujone, (1R)-(−)-myrtenal, (1R)-(−)-myrtenol, (R)-(+)-pulegone, (R)-(−)-carvone, (S)-(−)-citronellal, (S)-(−)-perillaldehyde, (±)-linalool, (±)-nerolidol, (±)-α-bisabolol, 3-Carene, 7-Oxabicylo-(2-2-1)heptane, alpha Pinene, alpha Terpineol, Ascaridole, carvacrol, Carvone, citral, cyclohexanemethanol, Cyclopentene oxide, d-Limonene. EUCALYPTOL, eucarvone, farnesol, Fenchone, Fennel oil, geraniol, L-(−)-menthol, Limonene oxide, menthone, myrcene, nerol, Nerolidol, ocimene, octisalate, Peppermint oil, phytol, Pinene oxide, piperitone, Pulegone, retinoic acid, retinol, squalene, Terpinen-4-ol, terpinolene, thymol, α-humulene, α-phellandrene, β-carotene, β-citronellol, and combinations thereof.

In another aspect, the at least one terpene can have a c Log P of greater than 2.0. In one example, the at least one terpene is a member selected from the group consisting of (+)-aromadendrene, (+)-cedrol, (+)-cedryl acetate, (+)-dihydrocarveol, (+)-dihydrocarvone, (+)-longifolene, (+)-β-cedrene, (−)-carveol, (−)-caryophyllene oxide, (−)-dihydrocarveol, (−)-epiglobulol, (−)-guaiol, (−)-isolongifolol, (−)-isopulegol, (−)-trans-caryophyllene, (−)-α-cedrene, (−)-α-thujone, (R)-(+)-pulegone, (R)-(−)-carvone, (S)-(−)-citronellal, (S)-(−)-perillaldehyde, (±)-linalool, (±)-nerolidol, (±)-α-bisabolol, 3-Carene, alpha Pinene, alpha Terpineol, Ascaridole, carvacrol, Carvone, citral, d-Limonene, EUCALYPTOL, eucarvone, farnesol, Fenchone, Fennel oil, geraniol, L-(−)-menthol, Limonene oxide, menthone, myrcene, nerol, Nerolidol, ocimene, octisalate, Peppermint oil, phytol, piperitone, Pulegone, retinoic acid, retinol, squalene, Terpinen-4-ol, terpinolene, thymol, α-humulene, α-phellandrene, β-carotene, -citronellol, and combinations thereof.

In another aspect, the at least one terpene can have a c Log P of greater than 3.0. In one example, the at least one terpene can be a member selected from the group consisting of: (+)-aromadendrene, (+)-cedrol, (+)-cedryl acetate, (+)-longifolene, (+)-β-cedrene, (−)-caryophyllene oxide, (−)-epiglobulol, (−)-guaiol, (−)-isolongifolol, (−)-trans-caryophyllene, (−)-α-cedrene, (S)-(−)-citronellal, (±)-linalool, (±)-nerolidol, (±)-α-bisabolol, citral, d-Limonene, farnesol, geraniol, myrcene, nerol. Nerolidol, phytol, retinoic acid, retinol, squalene, terpinolene, α-humulene, β-carotene, β-citronellol, and combinations thereof.

In another example, the at least one terpene can be a member selected from the group consisting of: biasbolol, β-caryophyllene, D-limonene, linalool, nerolidol, myrcene, terpinolene, and combinations thereof.

In another example, the at least one terpene can be a member selected from the group consisting of: terpinolene, D-limonene, (−)-trans-caryophyllene, borneol, camphor, carvacol, carvone, cineole, cymene, eugenol, farnesol, geranuol, humulene, isopulegol decanoate and combinations thereof.

In another aspect, the first penetration enhancer, the second penetration enhancer, or both can be amphiphilic.

In one example, the first penetration enhancer, the second penetration enhancer, or both can be selected from the group consisting of: limonene, linalool, menthol, and combinations thereof.

In another aspect, the first penetration enhancer, the second penetration enhancer, or both can have a c Log P of greater than one or more of: 0, 1, 2, 3, 5, or a combination thereof; and the first penetration enhancer, the second penetration enhancer, or both can have a polar surface area of greater than one or more of: 20, 35, 50, or a combination thereof.

In another aspect, the first penetration enhancer, the second penetration enhancer, or both can comprise one or more of: C2 to C4 alcohols, C2 to C4 diols, triacetin, glycerol monooleate, glycerol monolaurate, oleic alcohol, lauryl alcohol, isopropyl myrisate, sorbitan esters, or combinations thereof.

In another aspect, the first penetration enhancer, the second penetration enhancer, or both can be an ether or an ester.

In another aspect, the first penetration enhancer, the second penetration enhancer, or both can be selected from the group consisting of: dimethyl isosorbide, diethylene glycol monoethyl ether, ethylene glycol monododecyl ether, adipic acid diisopropyl ester, diethyl sebacate, and combinations thereof.

In another aspect, the second penetration enhancer can be dimethyl isosorbide or isosorbide mononitrate.

In one aspect, the at least one cannabinoid receptor modulator and the dimethyl isosorbide or isosorbide mononitrate can be present in a ratio of from about 20:1 to about 1:20. In another example, the first penetration enhancer and the dimethyl isosorbide or isosorbide mononitrate can be present in a ratio of from about 1:10 to about 10:1.

In another aspect, the first penetration enhancer can comprise a 16-carbon saturated fatty acid.

In another aspect, the first penetration enhancer can comprise one or more of a hexadecenoic acid, a salt of hexadecenoic acid, an ester of hexadecenoic acid, or a combination thereof.

In another aspect, the first penetration enhancer can comprise a member selected from the group consisting of: propan-2-yl hexadecanoate, 2-(3,4-dihydroxyoxolan-2-yl)-2-hydroxyethyl hexadecanoate, (6-{[3,4-dihydroxy-2,5-bis(hydroxymethyl)oxolan-2-yl]oxy}-3,4,5-trihydroxyoxan-2-yl)methyl hexadecanoate, hexadecenoic acid, 2-(2-oxoazepan-1-yl)hexadecenoic acid, 1-(2,6-dimethylmorpholin-4-yl)hexadecan-1-one, 1-(4-hexadecanoylpiperazin-1-yl)hexadecan-1-one, 1-(4-methylpiperazin-1-yl)hexadecan-1-one, 1-(azepan-1-yl)hexadecan-1-one, 1-(morpholin-4-yl)hexadecan-1-one, 1-(piperazin-1-yl)hexadecan-1-one, hexadecan-1-ol, or combinations thereof.

In another aspect, the first penetration enhancer can be cetyl palmitate, sorbitan oleate, sorbitan palmitate, sorbitan monopalmitate, sorbitan stearate or ascorbyl palmitate, or a combination thereof.

In one aspect, the second penetration enhancer can be present in the topical composition at a concentration of from about 0.0001 wt % to about 50 wt %. In one example, the second penetration enhancer can be present in the topical composition at a concentration of from about 2 wt % to about 20 wt %. In another example, the second penetration enhancer can be present in the topical composition at a concentration of from about 4 wt % to about 12 wt %.

In another aspect, the first penetration enhancer can be present in the topical composition at a concentration of from about 0.0001 wt % to about 50 wt %. In one example, the first penetration enhancer can be present in the topical composition at a concentration of from about 1 wt % to about 10 wt %. In another example, the first penetration enhancer can be present in the topical composition at a concentration of from about 1 wt % to about 6 wt %.

In another aspect, the topical composition can further comprise an additional penetration enhancer.

In another aspect, the at least one cannabinoid receptor modulator can be present in the topical composition at a concentration of from about 0.0001 wt % to about 50 wt %. In one example, the at least one cannabinoid receptor modulator can be present in the topical composition at a concentration of from about 0.1 wt % to about 10 wt %. In another example, the at least one cannabinoid receptor modulator can be present in the topical composition at a concentration of from about 0.1 wt % to about 5 wt %.

In another aspect, the at least one cannabinoid receptor modulator can have a potency that is increased by at least 10% relative to the potency of the at least one cannabinoid receptor modulator without the penetration enhancer, the penetration additive, or a combination thereof.

In another aspect, the cannabinoid receptor modulator can comprise a cannabinoid, a fatty acid amide, or a combination thereof. In one example, the cannabinoid can comprise a phytocannabinoid extract. In one example, the phytocannabinoid extract can comprise one or more of: a cannabidiol (CBD) component, a tetrahydrocannabinol (T-C) component, a cannabigerol (CBG) component, a cannabinol (CBN) component, a cannabichromene (CBC) component, a cannabielsoin (CBE) component, a cannabifuran (CBF) component, a cannabicyclol (CBL) component, a cannabitriol (CBT) component, a cannabinodivarin (CBV) component, a cannabiripsol (CBR) component, a cannabicitran (CBT-C) component, a cannabiglendol-C3 component, or combinations thereof.

In one example, the CBD component can be a member selected from the group consisting of: CBD, cannabidiolic acid (CBDA), cannabidiorcol (CBDC1), cannabidiol monomethylether (CBDM), cannabidivarin (CBDV), cannabidivarinic acid (CBDVA), and combinations thereof.

In another example, the THC component can be a member selected from the group consisting of: THC, 10-oxo-delta-6a-tetrahydrocannabinol (OTHC), delta-8-tetrahydrocannabinolic acid (Ei8-THCA), delta-8-tetrahydrocannabinol (d8THC. (Ei8-THC), delta-9-tetrahydrocannabinol (d9THC, Gi9-THC), delta-9-tetrahydrocannabinol-C4 (THC-C4), delta-9-tetrahydrocannabinolic acid A ((Ei9-THCA, THCA-A), delta-9-tetrahydrocannabinolic acid B (Ri9-THCB, THCA-B), delta-9-tetrahydrocannabiorolic acid (Gi9-THCA-C1, THCA-C1), delta-9-tetrahydrocannabinolic acid C4 ((E19-THC-C4, THCA-C4), delta-9-tetrahydrocannabiorcol (Ei9-THC-C1), delta-9-tetrahydrocannabivarin (THCV), delta-9-tetrahydrocannabivarinic acid (THCVA), trihydroxy-delta-9-tetrahydrocannabinol (TRIOH-THC), and combinations thereof.

In another example, the CBG component can be a member selected from the group consisting of: CBG, cannabigerolic acid (CBGA), cannabigerolic acid monomethylether (CBGAM), cannabigerovarin (CBGV), cannabigerovarinic acid (CBGVA), cannabigerol monomethylether (CBGM), and combinations thereof.

In another example, the CBN component can be a member selected from the group consisting of: CBN, cannabinolic acid (CBNA), cannabidiorcol (CBN-C1), cannabinol-C2 (CBN-C2), cannabinol-C4 (CBN-C4), cannabinodiol (CBND), cannabinol methylether (CBNM), delta-9-cis-tetrahydrocannabinol (CIS-THC), and combinations thereof.

In another example, the CBC component can be a member selected from the group consisting of: CBC, cannabichromenic acid (CBCA), cannabichromanon (CBCN), cannabichromencvarin (CBCV), cannabichromevarinic acid (CBCVA), and combinations thereof.

In another example, the CBE component can be a member selected from the group consisting of: CBE, cannabielsoic acid A (CBEA-A), cannabielsoic acid B (CBEA-B), and combinations thereof.

In another example, the CBF component can be a member selected from the group consisting of: CBF, dehydrocannabifuran (CBFD), and combinations thereof.

In another example, the CBL component can be a member selected from the group consisting of: CBL, cannabicyclolic acid (CBLA), cannabicyclovarin (CBLV), and combinations thereof.

In another example, the CBT component can be a member selected from the group consisting of: CBT, cannabitriolvarin (CBTV), 10-Ethoxy-9-hydroxy-delta-6a-tetrahydrocannabinol, 8,9-Dihydroxy-delta-6a-tetrahydrocannabinol, and combinations thereof.

In another example, the CBV component can be a member selected from the group consisting of: CBV, cannabinodivarin (CBVD), and combinations thereof.

In another example, the CBR component can comprise CBR.

In another example, the CBT-C component can comprise CBT-C.

In another example, the cannabiglendol-C3 component can comprise cannabiglendol-C3.

In another aspect, the cannabinoid can comprise a synthetic cannabinoid.

In one example, the synthetic cannabinoid can comprise one or more of: an adamantoylindole, a benzimidaole, a benzoylindole, a cyclohexylphenol, a dibenzopyran, a dibenzopyran hybrid, an eicosanoid, a hydrocarbon, an indazole carboxamide, an indazole-3-carboxamide, an indole-3-carboxamide, an indole-3-carboxylate ester, a naphthoylindazole, a naphthoylindole, a naphthoylpyrrole, a naphthylmethylindene, a naphthylmethylindole, phenylacetylindole, a pyrazolecarboxamide, a tetramethylcyclopropylcarbonylindazole, a tetramethylcyclopropanoylindole, a quinolinyl ester, or combinations thereof.

In one aspect, the cannabinoid can comprise an endocannabinoid.

In one example, the endocannabinoid can comprise one or more of: anandamide (ANA), 2-arachidonoylglycerol (2-AG), 2-arachidonyl glyceryl ether, N-arachidonoyl dopamine (NADA), virodhamine (OAE), lysophosphatidylinositol (LPI), or combinations thereof.

In another aspect, the topical composition may not comprise a psychoactive cannabinoid.

In another aspect, the fatty acid amide can comprise one or more of an N-acylethanolamine, a fatty acid primary amide (FAPA), or a combination thereof.

In one example, the N-acylethanolamine can be a member selecting from the group consisting of: anandamide, N-palmitoylethanolamine, N-oleoylethanolamine, N-stearoylethanolamine, N-docosahexaenoylethanolamine, N-docosatetraenoylethanolamine, N-eicosapentaenoylethanolamide, and combinations thereof.

In another example, the FAPA can be oleamide.

In one aspect, the first penetration enhancer, the second penetration enhancer, or both can comprise a secondary active agent, or the secondary active agent can be different from the first or second penetration enhancer.

In one aspect, the secondary active agent can be a member selected from the group consisting of: an analgesic, an anti-inflammatory agent, an anti-infective agent, an antioxidant, a catalase, an anti-tumor agent, an anesthetic, an anti-rheumatic agent, a growth factor, a cytokine, an amino acid, a protein, a vaccine, a hormone, a vitamin, and combinations thereof.

In another aspect, the secondary active agent can be present at a concentration of from about 0.0001 wt % to about 50 wt %.

In one example, the analgesic can be present in the topical composition at a concentration of from about 1 wt % to about 35 wt %.

In another example, the analgesic can be a member selected from the group consisting of palmitoylethanolamide, β-caryophyllene, histamine, dihydrochloride, niaouli oil, myrcene, menthol, and combinations thereof.

In one example, the anti-inflammatory agent can be present in the topical composition at a concentration of from about 1 wt % to about 20 wt %.

In another example, the anti-inflammatory agent can be a member selected from the group consisting of: palmitoylethanolamide, bisabolol, linalool, myrcene, and combinations thereof.

In one example, the anti-infective agent can be present in the topical composition at a concentration of from about 1 wt % to about 15 wt %.

In another example, the anti-infective agent can be a member selected from the group consisting of: bisabolol, β-caryophyllene, niaouli (Melaleuca alternifolia) oil, and combinations thereof.

In one example, the antioxidant can be present in the topical composition at a concentration of from about 0.1 wt % to about 5 wt %.

In another example, the antioxidant can be a member selected from the group consisting of: D-limonene, terpinolene, eucalyptol and combinations thereof.

In one example, the pharmaceutically acceptable carrier can include at least one of an emulsifier, a thickener, an emollient, a preservative, a solubilizing agent, a tonicity agent, a buffer, a pH adjuster, water, and combinations thereof.

In one example, the emulsifier can be present in the topical composition at a concentration of from about 0.0001 wt % to about 50 wt %. In another example, the emulsifier can be present in the topical composition at a concentration of from about 10 wt % to about 30 wt %.

In another example, the emulsifier can be a member selected from the group consisting of octanoic acid triglyceride, decanoic acid triglyceride, cetearyl olivate, sorbitan olivate, sorbitan gum powder, biopolysan 220B, lecithin, ceteryl wheat straw glucosides, ceterayl alcohol, and combinations thereof.

In one example, the thickener can be present in the topical composition at a concentration of from about 0.0001 wt % to about 50 wt %. In another example, the thickener can be present in the topical composition at a concentration of from about 1 wt % to about 10 wt %. In another example, the thickener can be a polymer.

In another example, the thickener can be a member selected from the group consisting of cetearyl olivate, sorbitan olivate, polyacrylate crosspolymer-6, carbomer, locust bean gum, xanthan gum, gelatin, and combinations thereof.

In one example, the emollient can be present in the topical composition at a concentration of from about 1 wt % to about 50 wt %. In another example, the emollient can be present in the topical composition at a concentration of from about 4 wt % to about 20 wt %.

In another example, the emollient can be a member selected from the group consisting of cetearyl olivate, sorbitan olivate, biopolysan 220B, Helianthus annus seed oil, laurel laurate, cetyl palmitate, and combinations thereof.

In one example, the preservative can be a member selected from the group consisting of: biopolysan 220B, and combinations thereof.

In one aspect, the topical composition can have a tonicity of from about 250 mOsm/kg to about 350 mOsm/kg.

In one aspect, the topical composition can have a pH of from about 6.3 to about 7.8.

In one aspect, the topical composition can have a viscosity of from about 1 cP to about 1000 cP.

In another aspect, the topical composition can be formulated as one of: a solution, a suspension, an emulsion, a gel, a hydrogel, a thermo-responsive gel, a cream, a lotion, an ointment, a liniment, a paste, a mousse, an aerosol, a spray, a wax, a balm, a suppository, an adhesive, erodible matrix, a liquid reservoir, a patch, or a combination thereof.

In one aspect, the pharmaceutically acceptable carrier can comprise a biocompatible polymer.

In one example, the biocompatible polymer can be a member selected from the group consisting of: rubbers, silicone polymers and copolymers, acrylic polymers and copolymers, and combinations thereof.

In one aspect, the pharmaceutically acceptable carrier can comprise a viscous material suitable for use as a liquid reservoir. In one example, the viscous material can form a gel.

In one aspect, the patch can be a transdermal patch. In one example, the transdermal patch can be a transdermal matrix patch. In another example, the transdermal patch can be a liquid reservoir patch.

In one aspect, the combination can facilitate an extended therapeutic effect. In one example, the extended therapeutic effect can have a duration of from about 5 seconds to about one week. In one example, the extended therapeutic effect can have a duration of from about 30 minutes to about 5 days. In one example, the extended therapeutic effect can have a duration of from about 4 hours to about 3 days.

In another aspect, the combination can facilitate a rapid therapeutic effect that commences within a selected time. In one example, the selected time can be less than one or more of 1 second, 5 seconds, 30 seconds, 2 minutes, 5 minutes, 15 minutes, 30 minutes, 1 hour, 4 hours, 8 hours, 24 hours, or a combination thereof.

In another example, the combination can facilitate an extended therapeutic effect or a rapid therapeutic effect for the topical composition relative to a therapeutic effect duration or a therapeutic effect onset time for a topical composition without the combination.

In another example, the combination can facilitate a substantially equivalent therapeutic effect duration or therapeutic effect onset time for a reduced dose of the cannabinoid receptor modulator relative to a therapeutic effect duration or a therapeutic effect onset time for an increased dose of the cannabinoid receptor modulator for a topical composition without the combination, wherein the reduced dose is less than the increased dose by one or more of 5%, 10%, 20%, 35%, 50%, or a combination thereof.

In another example, the combination can facilitate a substantially equivalent therapeutic effect duration or therapeutic effect onset time for a reduced daily dose of the cannabinoid receptor modulator relative to a therapeutic effect duration or a therapeutic effect onset time for an increased daily dose of the cannabinoid receptor modulator for a topical composition without the combination, wherein the reduced daily dose is less than the increased daily dose by one or more of: 5%, 10%, 20%, 35%, 50%, or a combination thereof.

In another example, the combination can facilitate a substantially equivalent therapeutic effect duration or therapeutic effect onset time for a reduced dose frequency of the cannabinoid receptor modulator relative to a therapeutic effect duration or a therapeutic effect onset time for an increased dose frequency of the cannabinoid receptor modulator for a topical composition without the combination, wherein the reduced dose frequency is less than the increased dose frequency by one or more of: once per week, twice per week, three time per week, once per day, twice per day, three times per day, five times per day, or a combination thereof.

In another embodiment, a method of treating a condition in a subject that is responsive to treatment with a cannabinoid receptor modulator can comprise: administering a therapeutically effective amount of the topical composition to the subject.

In one aspect, the condition can be pain. In one example, the pain can be one or more of acute pain, chronic pain, neuropathic pain, nociceptive pain, radicular pain, or combinations thereof. In another example, the pain can be one or more of joint pain, muscle pain, arthritis, fibromyalgia, or a combination thereof. In another example, the pain can be induced by one or more of peripheral nerve damage, inflammation, cancer, cancer treatment, burns, surgery, or a combination thereof.

In another aspect, the condition can be edema.

In another aspect, the condition can be inflammation.

In another aspect, the condition can be one or more of a neurological condition, a psychological condition, a digestive condition, a heart condition, a kidney condition, a bladder condition, a liver condition, a skin condition, an immune disorder, an endocrine disorder, a breathing disorder, or combinations thereof.

In another aspect, the condition can comprise one or more of: Alzheimer's disease, dementia, amyotrophic lateral sclerosis (ALS), epilepsy, seizures, multiple sclerosis, muscle spasms, Parkinson's disease, stroke, brain injury, headaches, migraines, or combinations thereof.

In another aspect, the condition can comprise one or more of: a sleep disorder, anxiety, depression, ADD, ADHD, autism spectrum disorder, schizophrenia, psychosis, bipolar disorder, addiction, alcoholism, post-traumatic stress disorder, stress, or combinations thereof.

In another aspect, the condition can comprise one or more of: HIM Crohn's disease, glaucoma, cancer, diabetes, osteoporosis, nausea, obesity, antibiotic resistance, prion disease, endometriosis, chronic fatigue syndrome, or combinations thereof.

In another aspect, the treatment can provide a reduction in symptoms of at least 10% within a selected amount of time after administration.

In another aspect, symptoms of the condition can be reduced by at least 10% since commencement of the treatment.

In another aspect, the topical composition can comprise a dosage form having from about 25 ul to about 10 ml of the cannabinoid receptor modulator.

In another aspect, a daily dose of the topical composition can be from about 25 ul to about 10 ml of the cannabinoid receptor modulator.

In another aspect, the method can further comprise administering the therapeutically effective amount of the topical composition to the subject 1 to 5 times per day.

In another aspect, the method can further comprise administering the therapeutically effective amount of the topical composition to the subject according to a dosage regimen of at least once per day for a duration of from about a single day to about 3 months.

In one aspect, the therapeutically effective amount can facilitate an extended therapeutic effect. In one example, the therapeutically effective amount can have a duration of from about 5 seconds to about one week. In another example, the therapeutically effective amount can have a duration of from about 30 minutes to about 5 days. In one example, the therapeutically effective amount can have a duration of from about 4 hours to about 3 days.

In another aspect, the therapeutically effective amount can facilitate a rapid therapeutic effect that commences within a selected time. In one example, the selected time can be less than one or more of 1 second, 5 seconds, 30 seconds, 2 minutes, 5 minutes, 15 minutes, 30 minutes, 1 hour, 4 hours, 8 hours, 24 hours, or a combination thereof.

In one aspect, the therapeutically effective amount can facilitate an extended therapeutic effect or a rapid therapeutic effect for the topical composition relative to a therapeutic effect duration or a therapeutic effect onset time for a topical composition without the combination.

In another aspect, the therapeutically effective amount can facilitate a substantially equivalent therapeutic effect duration or therapeutic effect onset time for a reduced dose of the cannabinoid receptor modulator relative to a therapeutic effect duration or a therapeutic effect onset time for an increased dose of the cannabinoid receptor modulator for a topical composition without the combination, wherein the reduced dose is less than the increased dose by one or more of: 5%, 10%, 20%, 35%, 50%, or a combination thereof.

In one aspect, the therapeutically effective amount can facilitate a substantially equivalent therapeutic effect duration or therapeutic effect onset time for a reduced daily dose of the cannabinoid receptor modulator relative to a therapeutic effect duration or a therapeutic effect onset time for an increased daily dose of the cannabinoid receptor modulator for a topical composition without the combination, wherein the reduced daily dose is less than the increased daily dose by one or more of: 5%, 10%, 20%, 35%, 50%, or a combination thereof.

In another aspect, the therapeutically effective amount can facilitate a substantially equivalent therapeutic effect duration or therapeutic effect onset time for a reduced dose frequency of the cannabinoid receptor modulator relative to a therapeutic effect duration or a therapeutic effect onset time for an increased dose frequency of the cannabinoid receptor modulator for a topical composition without the combination, wherein the reduced dose frequency is less than the increased dose frequency by one or more of: once per week, twice per week, three time per week, once per day, twice per day, three times per day, five times per day, or a combination thereof.

In another embodiment, a method of enhancing transdermal penetration of a cannabinoid receptor modulator can comprise: combining an amount of the cannabinoid receptor modulator with a combination of first and second penetration enhancers at synergistic ratios that facilitate a greater combined therapeutic effect than provided by a sum of individual therapeutic effects of the individual components of the combination. In one aspect, the cannabinoid receptor modulator can be present in the topical composition at a concentration of from about 0.0001 wt % to about 50 wt %.

It is understood that the above-described various types of compositions, dosage forms and/or modes of applications am only illustrative of embodiments of the present disclosure. Numerous modifications and alternative arrangements may be devised by those skilled in the art without departing from the spirit and scope of the present disclosure and the appended claims are intended to cover such modifications and arrangements. Thus, while the present disclosure has been described above with particularity and detail in connection with what is presently deemed to be the most practical and embodiments of the disclosure, it will be apparent to those of ordinary skill in the art that variations including, but not limited to, variations in size, materials, shape, form, function and manner of operation, assembly and use may be made without departing from the principles and concepts set forth herein.

1. A topical composition comprising:
a therapeutically effective amount of at least one cannabinoid receptor modulator;
an amount of a combination of a first and second penetration enhancer at synergistic ratios that facilitate a greater combined therapeutic effect than provided by a sum of individual therapeutic effects of the individual components of the combination; and
a pharmaceutically acceptable carrier suitable for topical administration.

2. The topical composition of claim 1, wherein the at least one cannabinoid receptor modulator and the combination of first and second penetration enhancers are present in a ratio of from about 20:1 to about 1:500.

3. The topical composition of claim 1, wherein the first and second penetration enhancers are present in a ratio of from about 10:1 to about 1:50.

4-18. (canceled)

19. The topical composition of claim 1, wherein:
the first penetration enhancer, the second penetration enhancer, or both has a c Log P of greater than one or more of: 0, 1, 2, 3, 5, or a combination thereof; and
the first penetration enhancer, the second penetration enhancer, or both has a polar surface area of greater than one or more of: 20, 35, 50, or a combination thereof.

20-35. (canceled)

36. The topical composition of claim 1, further comprising an additional penetration enhancer.

37-39. (canceled)

40. The topical composition of claim 1, wherein the at least one cannabinoid receptor modulator has a potency that is increased by at least 10% relative to the potency of the at least one cannabinoid receptor modulator without the penetration enhancer, the penetration additive, or a combination thereof.

41-64. (canceled)

65. The topical composition of claim 1, wherein:
the first penetration enhancer, the second penetration enhancer, or both comprise a secondary active agent, or
the topical composition further comprises the secondary active agent that is different from the first or second penetration enhancer.

66-75. (canceled)

76. The topical composition of claim 1, wherein the pharmaceutically acceptable carrier includes at least one of an emulsifier, a thickener, an emollient, a preservative, a solubilizing agent, a tonicity agent, a buffer, a pH adjuster, water, and combinations thereof.

77-88. (canceled)

89. The topical composition of claim 1, wherein the topical composition has a pH of from about 6.3 to about 7.8.

90. The topical composition of claim 1, wherein the topical composition has a viscosity of from about 1 cP to about 1000 cP.

91. The topical composition of claim 1, wherein the topical composition is formulated as one of: a solution, a suspension, an emulsion, a gel, a hydrogel, a thermo-responsive gel, a cream, a lotion, an ointment, a liniment, a paste, a mousse, an aerosol, a spray, a wax, a balm, a suppository, an adhesive, erodible matrix, a liquid reservoir, a patch, or a combination thereof.

92. The topical composition of claim 1, wherein the pharmaceutically acceptable carrier comprises a biocompatible polymer.

93-108. (canceled)

109. A method of treating a condition in a subject that is responsive to treatment with a cannabinoid receptor modulator, comprising:
administering a therapeutically effective amount of the topical composition of claim 1 to the subject.

110. The method of claim 109, wherein the condition is pain.

111-113. (canceled)

114. The method of claim 109, wherein the condition is edema.

115. The method of claim 109, wherein the condition is inflammation.

116-119. (canceled)

120. The method of claim 109, wherein the treatment provides a reduction in symptoms of at least 10% within a selected amount of time after administration.

121. The method of claim 109, wherein symptoms of the condition are reduced by at least 10% since commencement of the treatment.

122. The method of claim 109, wherein the topical composition comprises a dosage form having from about 25 ul to about 10 ml of the cannabinoid receptor modulator.

123. The method of claim 109, wherein a daily dose of the topical composition is from about 25 ul to about 10 ml of the cannabinoid receptor modulator.

124. The method of claim 109, further comprising administering the therapeutically effective amount of the topical composition to the subject 1 to 5 times per day.

125-135. (canceled)

136. A method of enhancing transdermal penetration of a cannabinoid receptor modulator, comprising:
combining an amount of the cannabinoid receptor modulator with a combination of first and second penetration enhancers at synergistic ratios that facilitate a greater combined therapeutic effect than provided by a sum of individual therapeutic effects of the individual components of the combination.

137. The method of claim 136, wherein the cannabinoid receptor modulator is present in the topical composition at a concentration of from about 0.0001 wt % to about 50 wt %.
